(19) United States
(12) Patent Application Publication
(10) Pub. No.: US 2022/0193029 A1
(43) Pub. Date: Jun. 23, 2022

(54) BRYOSTATIN COMPOUNDS FOR ENHANCEMENT OF IMMUNOTHERAPY

(71) Applicant: The Board of Trustees of the Leland Stanford Junior University, Stanford, CA (US)

(72) Inventors: Paul Wender, Stanford, CA (US); Akira J. Shimizu, Stanford, CA (US); Clayton Hardman, Stanford, CA (US)

(21) Appl. No.: 17/599,207

(22) PCT Filed: May 19, 2020
(86) PCT No.: PCT/US2020/033638

§ 371 Date: Sep. 28, 2021

Related U.S. Application Data
(60) Provisional application No. 62/850,905, filed on May 21, 2019.

(51) Int. Cl. A61K 31/365

(52) U.S. Cl. CPC A61K 31/365

(57) ABSTRACT

Provided herein is the use of bryostatin agents to selectively enhance expression, translocation and/or cell surface presentation of an antigen in target cells of interest to modulate immunogenicity of the target cells. Aspects of the methods include, administering an effective amount of a bryostatin agent to a subject to modulate immunogenicity of target cells. The subject methods include a method of treating cancer, including administering to a subject an effective amount of a bryostatin agent to enhance cell surface antigen or neoantigen presentation on target cells of the subject, and administering to the subject a therapeutically effective amount of a therapeutic agent that specifically binds the cell surface antigen to treat the subject for cancer. Aspects of the subject methods also include use of the bryostatin agents to sensitize the target cells to clearance by the subject's immune system.

### CROSS-REFERENCE

This application claims the benefit of U.S. Provisional Patent Application No. 62/850,905, filed May 21, 2019, which application is incorporated herein by reference in its entirety.

### STATEMENT REGARDING FEDERALLY SPONSORED RESEARCH

This invention was made with Government support under contracts CA031845 and AI1124743 awarded by the National Institutes of Health. The Government has certain rights in the invention.

### INTRODUCTION

Bryostatin 1 is being advanced in the clinic for treating various diseases, disorders and conditions, including HIV/AIDS, Alzheimer's Disease (AD) and cancer. Bryostatin can be isolated (e.g., in 0.00014% yield) from the marine organism Bugula neritina, produced in minor amounts through biosynthesis. Chemical synthesis methods for various natural bryostatin compounds and bryostatin analogs are described by Wender et al. (see e.g., WO2018067382) that can provide access to sufficient material for clinical development.

Leukemias and lymphomas are difficult to treat cancers, having a 5-year survival rate of approximately 60%, and accounting for approximately 10% of cancer-related deaths. New treatments of interest being developed include chimeric antigen receptor-T cell therapy (CAR-T cell therapy) and various targeted therapies, e.g., antibody-based therapies. CAR-T cell therapy involves the use of adoptive cell transfer (ACT), a process which utilizes a patient's own cultured T cells. In CAR-T cell therapy, T cells are removed from a patient and genetically altered to express CARs directed towards antigens specific for a known cancer (e.g., a tumor), or the T cells can be genetically altered to express CARs in situ. Alternatively, the T cells are provided from a healthy donor and genetically altered to express CARs directed towards antigens specific for a known cancer. Following amplification ex vivo to a sufficient number, the autologous or allogenic cells are infused back into the patient, resulting in the antigen-specific destruction of the cancer.

The ability of HIV to establish a long-lived latent infection within resting CD4+ T cells leads to persistence and episodic resupply of the virus in patients treated with antiretroviral therapy (ART), thereby preventing eradication of the disease. Bryostatin can activate these latently infected cells, potentially leading to their elimination by virus-mediated cytopathic effects, the host's immune response and/or therapeutic strategies targeting cells actively expressing virus (see e.g., Marsden et al., “In vivo activation of latent HIV with a synthetic bryostatin analog effects both latent cell “kick” and “kill” in strategy for virus eradication”, PLOS Pathogens 13(9): e1006575). Elimination of latently infected cells when done in conjunction with ART to eliminate the active virus, represents a strategy for treatment interruption or eradication of the disease. Elimination of latently infected cells when done in conjunction with broadly neutralizing antibodies (bNAbs) to eliminate the active virus, may also represent a strategy for treatment interruption or eradication of the disease (see e.g., Borducchi et al., “Antibody and TLR7 agonist delay viral rebound in SHIV-infected monkeys,” Nature (2008) November; 563(7731): 360-364).

### SUMMARY

This disclosure provides the use of bryostatin 1 and analogs based on the bryostatin scaffold (“bryostatin agents”) as cell modulating agents. The subject bryostatin agents can be used to selectively enhance one or more of expression, translocation, cell surface presentation, and surface persistence, of an antigen in target cells of interest. Non-limiting examples of antigens in target cells of interest include, protein antigens, peptide antigens, neoantigens, and antigens derived from treatment of the target cells with mRNA. Provided herein are methods of modulating target cells in a subject. Aspects of the methods include, administering an effective amount of a bryostatin agent to a subject to modulate immunogenicity of target cells. Aspects of the subject methods include, contacting autologous or allogenic cells ex vivo with a bryostatin agent to modulate immunogenicity of the autologous or allogenic cells. The subject methods include, modulating the target cells for use in immunotherapy. The subject methods include, modulating the target cells for treatment of a disease. Non-limiting examples of diseases for treatment by the subject methods include, cancer, HIV, neurological disorders, dementia and Alzheimer's Disease. As such, the subject methods include a method of treating cancer, including administering to a subject an effective amount of a bryostatin agent to enhance cell surface antigen or neoantigen presentation on target cells of the subject, and administering to the subject a therapeutically effective amount of a therapeutic agent that specifically binds the cell surface antigen to treat the subject for cancer. The subject methods may include selectively enhancing cell surface presentation of target antigens or neoantigens, and selectively decreasing cell surface presentation of other antigens or neoantigens. Aspects of the subject methods also include use of the bryostatin agents to sensitize target cells to clearance by the subject's immune system.

### BRIEF DESCRIPTION OF THE FIGURES

FIG. 1, panels A-D, illustrate the synthetic strategy for preparation of bryostatin and analogs and binding structures with Protein Kinase C (PKC). Panel A: Retrosynthetic analysis of the bryostatin scaffold with pharmacophoric elements of the C-ring subunit identified as the C1 carbonyl, C19 hemiketal, and C26 alcohol. C13 (indicted with the sphere) is highlighted as an area of interest for analog synthesis. Panel B: Rendering of the PKC-bryostatin-membrane ternary complex. Bryostatin 1 shown inside the rectangular box. Pharmacophoric elements of the C-ring subunit of the bryostatin scaffold (see FIG. 1, panel A) interact directly with PKC (dark gray) while the A and B rings are imbedded in the plasma membrane (light gray). Panel C: Representative conformers of the bryostatin scaffold bound to PKC that fit experimentally determined intramolecular distances determined by REDOR NMR. Panel D: Convergent construction of the bryostatin scaffold from acid 1 and enal 2. C13 functionality provides a versatile starting point for late-stage diversification, avoiding interference with the pharmacophoric elements of the C ring (as identified in FIG. 1, panel A).

FIG. 2A-2B illustrates the therapeutic strategy of methods of treating cancer FIG. 2A is a schematic showing the effects of cytotoxic chemotherapeutics and FIG. 2B is a schematic showing selective mAb or CAR T-cell therapeutics.

FIG. 3, panels A-D, illustrate that CD22 site density of target cells limits CD22 CAR functionality. Panel A: Histogram of CD22 expression on CRISPR/Cas9 edited CD22 negative NALM6 B-ALL lines transduced to express varying levels of CD22. NALM6 refers to the parental cell line. Interferon gamma (Panel B) and IL-2 (Panel C) production by CD22 CAR transduced T cells upon coculture with NALM6 cell lines expressing varying CD22 site densities. *=p<0.05, ***=p<0.005, ****=p<0.001 by one way analysis of variance (ANOVA). Data shown in panels B and C is representative of 3 independent experiments. Lines represent means+/−standard error of measurement of triplicate wells. Panel D: Xenograft model demonstrating clearance of parental NALM6 by CD22 CAR T cells at a dose of 6×10⁶ per mouse (NOD SCID gamma (NSG)) administered at day 3 following leukemia injection but failure of the same CAR T cells to eradicate NALM6 expressing low CD22 site density despite initial delay in leukemia progression.

Representative of 3 independent experiments. Corresponds to FIG. 4 of Fry et al., Nature Medicine 2018, 24, 20-27.

FIG. 4, panels A-D shows that Bryostatin 1 (B1) and various exemplary bryostatin agents induce sustained CD22 surface expression in vitro in NALM6, JP and 2F7 cells.

FIG. 5 illustrates that bryostatin 1 administration to mice with CD22 CAR T cell treatment improves durability of response. NSG mice were injected with 1×106 GPF-positive NALM6 tumor cells on day 0. On day 3, 4×106 mock or CD22 CAR were injected for treatment. Mice were given bryostatin 1 or DMSO three times weekly for 2 weeks. Mice were imaged using IVIS™ technology and luciferin-D IP injections.

FIG. 6, panel A illustrates bryostatin agent-induced activation of PKC determined by monitoring PKCδ-GFP translocation to the plasma membrane using confocal microscopy. FIG. 6, panels B-D illustrate cytosolic fluorescence normalized to t=0 (time immediately prior to addition of compound to media) and plotted against time. Error bars excluded for clarity. Maximum translocation of PKCδ-GFP to the plasma membrane reported in Table 2.

FIG. 7A-7R depict biological data for exemplary bryostatin agents.

### DEFINITIONS

Before describing exemplary embodiments in greater detail, the following definitions are set forth to illustrate and define the meaning and scope of the terms used in the description.

Unless defined otherwise, all technical and scientific terms used herein have the same meaning as commonly understood by one of ordinary skill in the art to which this invention belongs. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Numeric ranges are inclusive of the numbers defining the range.

“Alkyl” refers to monovalent saturated aliphatic hydrocarbyl groups having from 1 to 20 carbon atoms and such as 1 to 10 carbon atoms, or 1 to 6, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups having from 1 to 20 and in some cases, 1 to 10, or 1 to 6, or 1 to 3 carbon atoms that are either straight-chained or branched, and which are optionally interrupted with one or more groups selected from —O—, —NR¹⁰—, —NR¹⁰C(O)—, —C(O)NR¹⁰— and the like, wherein R¹⁰ is selected from hydrogen, alkyl, substituted alkyl, aryl, and substituted aryl, as defined herein. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″— where R′ is alkyl group as defined herein and R″ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 20 carbon atoms and in some cases 2 to 10 carbon atoms, such as 2 to 7 carbon atoms, and having at least 1 and in some cases from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Allenyl” refers to straight chain or branched hydrocarbyl groups having from 2 to 20 carbon atoms and in some cases 2 to 10 carbon atoms, such as 2 to 7 carbon atoms and having a carbon atom having double bond unsaturation to each of its two adjacent carbon atoms. Included within this term are the stereo isomers or mixtures of these isomers.

The term “substituted allenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 20 carbon atoms and in some cases 2 to 10 carbon atoms, such as 2 to 7 carbon atoms, and having at least 1 and in some cases from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O)substituted alkyl, N R²⁰C(O)cycloalkyl, —NR²⁰C(O)substituted cycloalkyl, NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O— alkynyl, —O—C(O)O-substituted alkynyl, —O—C(O)O-aryl, —O—C(O)O-substituted aryl, —O—C(O)O— cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and in some cases from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO— substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, OSO₂-substituted alkyl, OSO₂-alkenyl, OSO₂-substituted alkenyl, OSO₂-cycloalkyl, OSO₂-substituted cycloalkyl, OSO₂-cycloalkenyl, OSO₂-substituted cylcoalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N2, —N₃, —SO₂R⁷⁰, —SO₂O M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻M⁺, —P(O)(OR⁷⁰) 2, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of 0, N and S, of which N may have —H or C₁-C₃ alkyl substitution; and each M⁺ is a counter ion with a positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁺M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁺M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂ O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “synthetic equivalent” or “reactive equivalent” is well understood by those skilled in the art, especially in the art of retrosynthesis, as a reference to a compound (or compounds) corresponding with a given “synthon” (E. J. Corey, Pure App. Chem., 1967, 14: 30-37). Any given synthon may have a plurality of synthetic equivalents. The term “synthon” refers to a compound that includes a core constituent part of a target molecule to be synthesized that is regarded as the basis of a synthetic procedure. For example, a synthon can refer to a fragment identified by retrosynthetic analysis or a synthetic building block related to a possible synthetic procedure. The term “synthetic equivalent” refers to a compound that can be utilized as an alternative to a target intermediate or starting material in a synthetic strategy without need for substantively changing the strategy and procedure. It is understood that a synthetic equivalent can be related to the target intermediate or starting material by including the same arrangement of functional groups or precursors thereof, or protected versions thereof, on a fragment of the underlying target scaffold of interest. Synthetic equivalents can refer to different functional groups having similar chemistry. A synthon can refer to a fragment resulting from retrosynthetic analysis e.g. disconnections of carbon-carbon bonds of the target molecule. A synthetic equivalent can refer to the actual substrates used in the synthetic procedure towards the target molecule. In some cases, the terms synthon and synthetic equivalent refer to the same molecule. In some cases, the term synthon refers to a synthetic fragment that allows for a plurality of synthetic equivalents. The definition of synthetic equivalent includes compounds, where a moiety of a compound of interest that would be labile or reactive under the conditions to be used in a said chemical reaction is protected or masked by an appropriate protecting group that can be cleaved off after said chemical reaction. In some cases, the definition includes compounds where a moiety of a compound of interest is protected or masked with a protecting group that is designed to be cleaved off during a said chemical reaction to provide a labile or reactive group in situ.

“Promoiety” refers to a form of protecting group that, when used to mask a functional group within an active agent, converts the active agent into a prodrug.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate (e.g., 3-hexyne-1,6-dioate), benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

The term “treating” or “treatment” as used herein means the treating or treatment of a disease or medical condition in a patient, such as a mammal (particularly a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient.

The term “contacting target cells” as used herein refers to dosing of target cells with a therapeutically effective amount of an agent, e.g., a bryostatin agent as described herein.

The term “clearing” or “clearance” in the context of “clearing the modulated target cells” or “clearance by the subject's immune system” refers to eradicating the modulated cells from the subject. In some cases, clearing of the modulated cells from the subject is achieved by administrating a therapeutically effective amount of a therapeutic. In some cases, contacting the target cells with a bryostatin agent (e.g., as described herein), is sufficient to clear the modulated cells from the subject. Accordingly, in some cases contacting the target cells with a bryostatin agent is sufficient to provide a therapeutic effect.

The term “cell surface antigen” refers to molecules that are located in a cell plasma membrane and at the cell surface which can be unique to the cell depending on the cell type and can be utilized to characterize, identify and target a cell. The immune system can recognize cell surface antigens. Cell surface antigen is meant to include neoantigens, i.e., newly formed antigens that have not been previously recognized by the immune system.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. In some cases, the cell is a T cell, which is converted to a CAR-T cell. In other cases, the cell is a NK cell, which is converted to a CAR-NK cell. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

“Expressed on” as used herein, may be used to describe a cellular moiety (e.g., proteins or complexes thereof), that is present on the surface of a cell, usually as a result of production of the cellular moiety, or a precursor thereof, in the cell and translocation of the cellular moiety, or a precursor thereof, to the extracellular surface of the plasma membrane of the cell.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc. In some cases, the individual is a human.

The term “donor” as used herein refers to a mammal, including, but not limited to, murines (e.g., rats, mice), lagomorphs (e.g., rabbits), non-human primates, humans, canines, felines, ungulates (e.g., equines, bovines, ovines, porcines, caprines), etc. from which target cells may be derived (e.g., allogenic cells). In some cases, the donor is a human. In some cases the donor is a healthy donor. In some cases the donor is a diseased donor.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

By “specifically binds” or “selectively bind” is meant that the molecule binds preferentially to the target of interest or binds with greater affinity to the target than to other molecules. For example, a DNA molecule will bind to a substantially complementary sequence and not to unrelated sequences. Specific binding may refer to non-covalent or covalent preferential binding to a molecule relative to other molecules or moieties in a solution or reaction mixture (e.g., an antibody specifically binds to a particular polypeptide or epitope relative to other available polypeptides). In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, or 10⁻¹⁶ M or less). “Affinity” refers to the strength of binding, increased binding affinity being correlated with a lower K_{D}.

The terms “antibody” and “immunoglobulin”, as used herein, are used interchangeably may generally refer to whole or intact molecules or fragments thereof and modified and/or conjugated antibodies or fragments thereof that have been modified and/or conjugated. The immunoglobulins can be divided into five different classes, based on differences in the amino acid sequences in the constant region of the heavy chains. All immunoglobulins within a given class will have very similar heavy chain constant regions. These differences can be detected by sequence studies or more commonly by serological means (i.e. by the use of antibodies directed to these differences). Immunoglobulin classes include IgG (Gamma heavy chains), IgM (Mu heavy chains), IgA (Alpha heavy chains), IgD (Delta heavy chains), and IgE (Epsilon heavy chains).

Antibody or immunoglobulin may refer to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized, see for instance Fundamental Immunology Ch. 7 (Paul, W., ed., 2nd ed. Raven Press, N.Y. (1989)). Briefly, each heavy chain typically is comprised of a heavy chain variable region (abbreviated as V_{H}) and a heavy chain constant region (abbreviated as C_{H}). The heavy chain constant region typically is comprised of three domains, C_{H}1, C_{H}2, and C_{H}3. Each light chain typically is comprised of a light chain variable region (abbreviated as V_{L}) and a light chain constant region (abbreviated herein as C_{L}). The light chain constant region typically is comprised of one domain, C_{L}. The V_{H} and V_{L} regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs).

Whole or largely intact antibodies are generally multivalent, meaning they may simultaneously bind more than one molecule of antigen whereas antibody fragments may be monovalent. Antibodies produced by an organism as part of an immune response are generally monospecific, meaning they generally bind a single species of antigen. Multivalent monospecific antibodies, i.e. antibodies that bind more than one molecule of a single species of antigen, may bind a single antigen epitope (e.g., a monoclonal antibody) or multiple different antigen epitopes (e.g., a polyclonal antibody).

Multispecific (e.g., bispecific) antibodies, which bind multiple species of antigen, may be readily engineered by those of ordinary skill in the art and, thus, may be encompassed within the use of the term “antibody” used herein where appropriate. Also, multivalent antibody fragments may be engineered, e.g., by the linking of two monovalent antibody fragments. As such, bivalent and/or multivalent antibody fragments may be encompassed within the use of the term “antibody”, where appropriate, as the ordinary skilled artisan will be readily aware of antibody fragments, e.g., those described below, which may be linked in any convenient and appropriate combination to generate multivalent monospecific or polyspecific (e.g., bispecific) antibody fragments.

Antibody fragments include but are not limited to antigen-binding fragments (Fab or F(ab), including Fab′ or F(ab′), (Fab)₂, F(ab′)₂, etc.), single chain variable fragments (scFv or Fv), “third generation” (3G) molecules, etc. which are capable of binding the epitopic determinant. These antibody fragments retain some ability to selectively bind to the subject antigen, examples of which include, but are not limited to:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab)₂, the fragment of the antibody that can be obtained by treating whole antibody with the enzyme pepsin without subsequent reduction;

(4) F(ab)₂ is a dimer of two Fab′ fragments held together by two disulfide bonds;

(5) Fv, defined as a genetically engineered fragment containing the variable region of the light chain and the variable region of the heavy chain expressed as two chains;

(6) Single chain antibody (“SCA”), defined as a genetically engineered molecule containing the variable region of the light chain, the variable region of the heavy chain, linked by a suitable polypeptide linker as a genetically fused single chain molecule; such single chain antibodies may be in the form of multimers such as diabodies, triabodies, tetrabodies, etc. which may or may not be polyspecific (see, for example, WO 94/07921 and WO 98/44001) and

(7) “3G”, including single domain (typically a variable heavy domain devoid of a light chain) and “miniaturized” antibody molecules (typically a full-sized Ab or mAb in which non-essential domains have been removed).

“Antigen-specific T cell” and “T cell that is specific to an antigen” as used herein, refer to a T cell expressing on its cell surface a T cell receptor (TCR) that specifically binds to an antigen by virtue of the structure of TCR polypeptides, such as the a and R polypeptide chains, containing variable regions. T cells whose TCR is specific to an antigen may have undergone recombination of the TCR genomic locus during maturation, and/or may have been genetically modified to express one or more TCR polypeptides or engineered TCR-like receptors (such as chimeric antigen receptors).

A “disease antigen” or “disease-associated antigen” refers to an epitope (e.g., an antigenic peptide, lipid, polysaccharide, nucleic acid, etc.) that elicits an immune response, such as a T-cell mediated immune response. Where the disease is a tumor, a tumor antigen or tumor-associated antigen may be an epitope expressed on a tumor cell. The tumor antigen may be unique to a tumor cell and not normally expressed on other cells of the body, particularly of the same lineage. In some cases, the tumor antigen may be an epitope normally expressed in other cells of the body, but does not induce an immune response in a non-tumor context. A tumor antigen may possess one or more epitopes that are typically expressed on normal cells during fetal development when the immune system is immature and unable to respond. A tumor antibody may possess one or more epitopes that are normally present at extremely low levels on normal cells but which are expressed at significantly higher levels on tumor cells.

Other definitions of terms may appear throughout the specification.

### DETAILED DESCRIPTION

This disclosure provides the use of bryostatin 1 and analogs based on the bryostatin scaffold (“bryostatin agents”) as cell modulating agents. The subject bryostatin agents can be used to selectively enhance one or more of, expression, translocation, cell surface presentation and cell surface persistence, of an antigen in target cells of interest. Non-limiting examples of antigens in target cells of interest include, protein antigens, peptide antigens, neoantigens, and antigens derived from treatment of the target cells with mRNA. Provided herein are methods of modulating target cells in a subject. Aspects of the methods include, administering an effective amount of a bryostatin agent to a subject to modulate the immunogenicity of target cells. Aspects of the subject methods include, contacting autologous or allogenic cells ex vivo with a bryostatin agent to modulate immunogenicity of the autologous or allogenic cells. The subject methods include, modulating the target cells for use in immunotherapy. The subject methods include, modulating the target cells for treatment of a disease. Non-limiting examples of diseases for treatment by the subject methods include, cancer, HIV, neurological disorders, dementia and Alzheimer's Disease. The subject methods include a method of treating cancer, including administering to a subject an effective amount of a bryostatin agent to enhance cell surface antigen or neoantigen presentation on target cells of the subject, and administering to the subject a therapeutically effective amount of a therapeutic agent that specifically binds the cell surface antigen to treat the subject for cancer. The subject methods may include selectively enhancing cell surface presentation of target antigens or neoantigens, and selectively decreasing cell surface presentation of other antigens or neoantigens. Aspects of the subject methods also include use of the bryostatin agents to sensitize the target cells to clearance by the subject's immune system.

Non-limiting examples of target cells to be modulated by the subject methods may include, diseased cells, infected cells, engineered cells, and normal antigen presenting cells. In some cases, the target cells are normal antigen presenting cells, and after treatment with a subject method (e.g., as described herein) are rendered more effective, exhibiting enhanced ability to clear diseased or target cells. For example, normal antigen presenting cells may be used in the subject methods to make a mRNA protein that would elicit an immune response, thus enhancing the immune system. In certain cases, non-limiting examples of target cells to be modulated by the subject methods may include, HIV infected cells, cancer cells, chimeric antigen receptor (CAR)-modified T cells (CAR-T cells) and chimeric antigen receptor-natural killer cells (CAR-NK). The subject bryostatin agents can be used in combination with chimeric antigen receptor-T cell therapy (CAR-T cell therapy) or CAR-NK cell therapy to improve patient response and prevent patient relapse driven by low and variable surface expression of an antigen on target cells of interest. CARs represent an emerging therapy for cancer (e.g., treatment of B and T cell lymphomas) and other malignancies. CAR-T cells can comprise patient-derived memory CD8+ T cells (e.g., autologous cells) modified to express a recombinant T cell receptor specific for a known antigen present on, for example, a tumor of interest. In this regard, T cells can be removed from a patient and modified to express CARs directed towards a specific antigen (e.g., on a tumor of interest), or the T cells can be modified to express CARs in vivo. CAR-T cells can also comprise donor-derived memory CD8+ T cells (e.g., allogenic cells derived from a donor) modified to express a recombinant T cell receptor specific for a known antigen present on, for example, a tumor of interest. While the present disclosure is generally described in the context of using CAR-T cell therapy for the treatment of cancer, it is to be understood that such therapy is not so limited.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

Methods

As summarized above, methods of the present disclosure provide use of a bryostatin agent to selectively enhance one or more of expression, translocation, cell surface presentation, and persistence, of an antigen (e.g., as described herein) in target cells of interest. As disclosed herein, target cells include diseased cells, infected cells, engineered cells, and normal antigen presenting cells. Provided herein are methods of modulating target autologous cells or allogenic cells ex vivo. Provided herein are methods of modulating target cells in vivo. Provided herein are methods of modulating target cells in a subject. Accordingly, the subject methods include, administering an effective amount of a bryostatin agent to a subject. In certain embodiments, the subject methods include a method of treating cancer, including administering to a subject an effective amount of a bryostatin agent to enhance cell surface antigen or neoantigen presentation on target cells of the subject, and administering to the subject a therapeutically effective amount of a therapeutic agent that specifically binds the cell surface antigen to treat the subject for cancer.

The present disclosure provides for use of bryostatin agents for the modulation of cell surface antigen presentation and persistence. In some cases, particular cell surface antigens can be targeted for selective modulation, e.g., selective enhancement of cell surface presentation. This disclosure provides for selective enhancement of target cell surface antigens and neoantigens over non-target antigens, e.g., cell surface antigens associated with global immune activation. Selective modulation can include enhancing expression or presentation of target antigens. In certain cases, the antigen is selected from a protein antigen, a peptide antigen, a neoantigen, and an antigen derived from treatment of the target cells with mRNA.

By “selectively” in the context of “selectively enhancing expression” of a protein in target cells of interest, or “selectively enhancing cell surface presentation” of a protein in target cells of interest, is used herein to refer to treatment of a target population of proteins so as to facilitate separation of members in the population having a desired attribute (e.g., a target antigen or neoantigen) from those that have a less desirable attribute. In other words, a particular member of the population of proteins in the target cells is preferentially enhanced (e.g., with respect to expression or surface presentation) to a greater extent than other proteins in the population.

By “enhancing expression”, or “enhancing expression of target antigens”, is meant that the expression of a target protein or antigen is increased by 50% or more. In some cases, the bryostatin agent increases cell surface presentation by 55% or more, such as 60% or more, 65% or more, 70% or more, 75% or more, 80% or more, 85% or more, 90% or more, 100% or more, or even more. In some cases, the bryostatin agent enhances expression by 2-fold or more, such as 3-fold or more, 4-fold or more, 5-fold or more, or even more. By “enhancing cell surface presentation” or “enhancing presentation of target antigens”, is mean that the presentation of a target protein or antigen is increased by 50% or more. In some case, the bryostatin agent increases cell surface presentation by 55% or more, such as 60% or more, 65% or more, 70% or more, 75% or more, 80% or more, 85% or more, 90% or more, 100% or more, or even more. In some cases, the bryostatin agent increases cell surface presentation by 2-fold or more, such as 3-fold or more, 4-fold or more, 5-fold or more, or even more. In some cases, the bryostatin agent will enhance the presentation and persistence of more than one target antigen, such as 2 or more target antigens, 3 or more target antigens, 4 or more target antigens, 5 or more target antigens or even more.

By “modulating target cells” or “modulating immunogenicity of target cells”, is meant to include enhancing the cell surface presentation of a particular protein and enhancing expression of a particular protein in the target cells. Modulating target cells can also include decreasing the expression or cell surface presentation of other proteins in the target cells. Modulating target cells includes modulation of cell surface antigen presentation and cell antigen expression. In some embodiments, the methods disclosed herein include modulating immunogenicity of target cells by selectively modulating a target antigen, e.g., by upregulating the target antigen. In some embodiments, the subject methods include modulating immunogenicity of target cells by downregulating other antigens, e.g., antigens associated with global immune activation. By “immunogenicity” is meant the ability to make the target cells more visible to the immune system of the subject, or the ability of the target cells to provoke an immune response by the subject.

The subject methods can be used in combination with chimeric antigen receptor-T cell therapy (CAR-T cell therapy) and chimeric antigen receptor-natural killer cell therapy (CAR-NK) to improve patient response and prevent patient relapse driven by low and variable surface expression of an antigen on target cells of interest. Treatment with CAR-T cell therapy has, in part, been limited by diminished and variable target antigen expression, the induction of antigen-specific toxicities targeting normal tissues expressing the target-antigen, and the extreme potency of CAR-T cell and/or CAR-NK cell treatments resulting in life-threatening cytokine-release syndromes. In particular, it has been observed that high affinity T cell receptor interactions with significant antigen burden can lead to activation-induced cell death.

Recently, preliminary Phase I clinical trial data for CD22-targeted CAR T therapy of patients with acute lymphoblastic leukemia (ALL) exhibited promising results, with approximately 70% of patients achieving a complete remission for a median duration of 6 months. However, despite this success, patient relapse was observed, driven by diminished and variable levels of CD22 surface expression. A mouse tumor xenograft model has indicated that critical CD22 surface activity is required for activation of anti-CD22 CAR T cells and tumor clearance (see, e.g., Fry et. al., Nat. Med., 2017, 24, 20-28).

Protein kinase C (PKC) modulators, C1 domain binders, and non-C1 domain targets that effect bryostatin's activities could serve as valuable adjuvants for targeted cancer therapy. Among the most studied PKC modulators, plant-derived phorbol esters (PEs) have been known to induce antigen presentation in a variety of cell lines. Bryostatin 1, a marine macrolide (see. e.g., Pettit et al. J. Am. Chem. Soc. 1982, 104 (24), 6846-6848; and Kortmansky et al. Cancer Invest. 2003, 21 (6), 924-936), can alter expression of surface antigens in tumor and other cell lines, making them more immunogenic and thus more susceptible to immune clearance. The data presented herein indicates that a bryostatin agent may be used in conjunction with CAR-T cell therapy to enhance the activity of CAR T cells by increasing the number and density of cell surface antigens on target cells.

Accordingly, provided herein are methods of modulating target cells in a subject. The method includes contacting target cells with an effective amount of a bryostatin agent to selectively enhance one or more of a) expression of an antigen in the target cells, b) translocation of an antigen in the target cells, c) cell surface presentation of an antigen in the target cells, and d) cell surface persistence of an antigen in the target cells, to modulate immunogenicity of the target cells. In some embodiments the antigen is a protein antigen. In some cases, the antigen is a peptide antigen. In some cases, the antigen is a neoantigen. In some other cases, the antigen is derived from treatment of the target cells with mRNA (e.g., an antigen derived from the delivery and expression of mRNA).

In some embodiments of the cell modulating methods, the target cells are HIV infected cells. In certain cases, the target cells are cells infected with latent HIV and modulating immunogenicity of the target cells comprises activating expression of HIV from the latent viral reservoir.

In some case, the bryostatin agent activates expression of HIV by 10% or more, such as 20% or more, 30% or more, 40% or more, 50% or more, 60% or more, 70% or more, 80% or more, 90% or more, or even more. In some cases, the bryostatin agent activates expression of HIV by 2-fold or more, such as 3-fold or more, 4-fold or more, 5-fold or more, or even more.

In some embodiments of the cell modulating methods, the contacting step is performed in vivo and comprises administering the bryostatin agent to a subject diagnosed with or suspected of having HIV. In certain cases, the subject cell modulating methods further include administering to the subject a therapeutically effective amount of a therapeutic that is capable of clearing the modulated target cells having activated expression of HIV. In some cases, the therapeutic capable of clearing the modulated target cells is antiretroviral therapy (ART). In some cases, the therapeutic capable of clearing the modulated target cells is a broadly neutralizing antibody (bNAb). In certain embodiments, the subject cell modulating methods include clearing the modulated target cells having activated expression of HIV without the administration of an additional therapeutic, e.g., bryostatin itself can be capable of clearing the modulated cells.

In some embodiments of the cell modulating methods, the target cells are chimeric antigen receptor (CAR)-modified T cells, or CAR-NK cells, and the contacting of the target cells with the bryostatin agent enhances expression or cell surface presentation of the CAR. In certain cases, the CAR has affinity for a target cell surface antigen selected from viral antigen, bacterial antigen, parasitic antigen, tumor cell associated antigen (TAA), disease cell associated antigen, an antigen derived from the treatment of the cells with mRNA, and any fragment thereof. In certain cases, the modified T cells are obtained from peripheral blood mononuclear cells, cord blood cells, a purified population of T cells, or a T cell line.

In some embodiments of the cell modulating methods, the contacting step is performed ex vivo and the target cells are derived from the subject to be treated (e.g., autologous cells). In other embodiments, the contacting step is performed ex vivo and the target cells are derived from a donor (e.g., allogenic cells).

In the subject methods, contacting the target cells ex vivo with a bryostatin agent can enhance CAR-T cell and (CAR)-NK cell production in the same manner in which it enhances antigen presentation in the target cells. In some cases, the enhancement for CARs is more efficient externalization of the recognition fragment that is engineered.

In some embodiments of the cell modulating methods, the target cells are selected from cancer cells, cancer stem cells, and cancer progenitor cells. In certain cases, the cancer cells are derived from a cancer including, but not limited to, breast cancer, prostate cancer, bladder cancer, soft tissue sarcoma, lymphomas, esophageal cancer, uterine cancer, bone cancer, adrenal gland cancer, lung cancer, thyroid cancer, colon cancer, glioma, liver cancer, pancreatic cancer, renal cancer, cervical cancer, testicular cancer, head and neck cancer, ovarian cancer, neuroblastoma and melanoma.

In some embodiments of the cell modulating methods, the contacting step is performed in vivo and comprises administering the bryostatin agent to a subject having cancer. The bryostatin agent can be administered via any convenient route, including orally, ocularly, aurally, subcutaneously, intravenously, intramuscularly, intradermally, intraperitoneally and inhalation.

In some cases, the bryostatin agent is administered subcutaneously. In some cases, the bryostatin agent is administered orally. In some cases, the bryostatin agent is administered ocularly. In some cases, the bryostatin agent is administered aurally. In some cases, the bryostatin agent is administered intravenously. In some cases, the bryostatin agent is administered intramuscularly. In some cases, the bryostatin agent is administered intradermally. In some cases, the bryostatin agent is administered intraperitoneally. In some cases, the bryostatin agent is administered by inhalation.

In some embodiments of the cell modulating methods, the method sensitizes the target cells to clearance by the subject's immune system. In some cases, the method sensitizes the target cells to clearance by the subjects innate immune system cells. In some cases, the method sensitizes the target cells to clearance by the subjects adaptive immune system cells.

By sensitizing the target cells to clearance by the subject's immune system, the cell modulating methods induce an immune response in the subject. In some cases, the cell modulating method does not affect the subject's own immune system cells, but is capable of selectively enhancing expression or cell surface presentation of an antigen for clearance by a subject's immune system.

In some embodiments of the cell modulating methods, the subject is relapsed to immune cell clearance and the bryostatin agent modulates T cell-mediated immune response to the target cell population. In some cases, the subject is refractory to immune cell clearance and the bryostatin agent modulates T cell-mediated immune response to the target cell population. In some cases, the bryostatin agent modulates the T-cell mediated immune response to the target cell population, such that the immune response to the target cell population is increased by 10% or more, 20% or more, 30% or more, 40% or more, 50% or more, 60% or more, 70% or more, 80% or more, 90% or more, or even more, compared to the untreated subject.

The term “relapse” or “relapsed” refers to the recurrence of illness after recovery. The term “refractory to a disease” refers to a subject being resistant or unresponsive to treatment for the particular disease. For example, refractory cancer, or resistant cancer is unresponsive to first and sometimes second line chemotherapy drugs, biological agents and/or radiation therapy.

Refractory cancer may shrink, but not to the point where the treatment is determined to be effective. In most cases, the tumor stays the same size it was before treatment (stable disease) or it grows (progressive disease).

In some embodiments of the cell modulating methods, the subject is receiving an immuno-oncology therapy. In certain cases, the subject is receiving a tumor antigen peptide vaccine.

In some embodiments of the cell modulating methods, the method further includes administering to the subject an effective amount of a therapeutic agent that is capable of one or more of inhibiting growth of the modulated target cells, and clearing the modulated target cells. In some cases, the method is used in combination with other therapies, wherein the combination results in an additive or synergistic benefit to the subject.

Also provided herein are methods of treating a subject for cancer, including administering to a subject an effective amount of a bryostatin agent to enhance cell surface antigen or neoantigen presentation on target cells of the subject; and administering to the subject a therapeutically effective amount of a therapeutic agent that specifically binds the cell surface antigen to treat the subject for cancer.

In some embodiments of the methods of treating cancer, the subject is relapsed to targeted anticancer therapy. In some cases, the subject is refractory to targeted anticancer therapy.

In some embodiments of the methods of treating cancer, the bryostatin agent sensitizes the target cancer cells to inhibition of growth by the therapeutic agent. In some cases, of the methods of treating cancer, the bryostatin agent sensitizes the target cancer cells to clearance by the therapeutic agent. In some embodiments of the methods of treating cancer, prior administering to a subject an effective amount of a bryostatin agent, the target cancer cells present cell surface antigen on the target cell surface at a therapeutically ineffective level (e.g., a level of presentation that is insufficient to induce cytotoxicity using the agent).

In some embodiments of the methods of treating cancer, the bryostatin agent enhances one or more of a) expression of cell surface antigens, b) translocation of expressed cell surface antigens to the target cell surface, and c) persistence of cell surface antigens on the target cell surface.

In some embodiments of the methods of treating cancer, the bryostatin agent enhances (increases) cell surface presentation of the cell surface antigen by 50% or more, such as 60% or more, 65% or more, 70% or more, 75% or more, 80% or more, 85% or more, 90% or more, 100% or more, or even more. In some cases, the cell surface presentation of the antigen is enhanced by 2-fold or more, 3-fold or more, 4-fold or more, 5-fold or more, or even more.

In some embodiments of the methods of treating cancer, cell surface antigen presentation on the target cancer cell is enhanced for 2 days or more after administration of the bryostatin agent. In some cases, cell surface antigen presentation on the target cancer cell is enhanced (increased) for 10 hours or more, 20 hours or more, 30 hours or more, 1 day or more, 2 days or more, 3 days or more, 4 days or more, 5 days or more, 6 days or more, 7 days or more, 8 days or more, 9 days or more, 10 days or more, or even more.

Accordingly, in some cases of the methods of treating cancer, the bryostatin agent enhances (increases) persistence of cell surface antigens on the target cell surface by 50% or more, such as 60% or more, 70% or more, 80% or more, 90% or more, or even more. In some cases, the cell surface presentation of the antigen is enhanced by 2-fold or more, 3-fold or more, 4-fold or more, 5-fold or more, or even more. In some cases, the bryostatin agent enhances (increases) persistence of cell surface antigens on the target cell surface for a period of 10 hours or more, 20 hours or more, 30 hours or more, 1 day or more, 2 days or more, 3 days or more, 4 days or more, 5 days or more, 6 days or more, 7 days or more, 8 days or more, 9 days or more, 10 days or more, or even more.

In some embodiments of the methods of treating cancer, the therapeutic agent that specifically binds the cell surface antigen is selected from, chimeric antigen receptor expressing T cells (CAR T-cells), chimeric antigen receptor expressing natural killer cells (CAR-NK cells), antibody agent, antibody drug conjugate (ADC) and bispecific antibody agent. In some cases, the therapeutic agent is CAR T-cells. In some cases, the therapeutic agent is CAR-NK cells. In some cases, the therapeutic agent is an antibody. In some cases, the therapeutic agent is an ADC. In other cases, the therapeutic agent is a bispecific antibody agent.

In some embodiments of the methods of treating cancer, administering to the subject a therapeutically effective amount of a therapeutic agent, includes administering to the subject a composition comprising a therapeutically effective amount of CAR T-cells that specifically bind the cell surface antigen present on a target cell population. In certain cases, the bryostatin agent modulates T cell-mediated immune response to the target cell population. In certain cases, the target cell population comprises tumor antigen selected from CD10, CD19, CD20, CD21, CD22, CD27, CD28, CD30, CD33, CD34, CD38, CD40, CD52, CD80, CD86, CD137, CDK4, CDK6, OX40 and CD340. In certain cases, the target cell population comprise tumor antigen CD22.

In some embodiments of the methods of treating cancer, the therapeutic agent that specifically binds the cell surface antigen is chimeric antigen receptor expressing T cells (CAR T-cells), or CAR-NK cells, and the CAR T-cells or CAR-NK cells are effective for treating B cell malignancy, CLL, ALL, B-ALL, Leukemia, Lymphoma or solid tumors. In certain cases, the solid tumors are selected from breast cancer, prostate cancer, bladder cancer, soft tissue sarcoma, lymphomas, esophageal cancer, uterine cancer, bone cancer, adrenal gland cancer, lung cancer, thyroid cancer, colon cancer, glioma, liver cancer, pancreatic cancer, renal cancer, cervical cancer, testicular cancer, head and neck cancer, ovarian cancer, neuroblastoma and melanoma.

In some embodiments of the methods of treating cancer, the bryostatin agent is administered prior to administration of the therapeutic agent that specifically binds the cell surface antigen to treat the subject for cancer. In some cases, administration of the bryostatin agent is prior to administration of the therapeutically effective amount of CAR-T cells. In some cases, administration of the bryostatin agent is prior to administration of the therapeutically effective amount of CAR-NK cells. In some cases, administration of the bryostatin agent is prior to administration of the therapeutically effective amount of an antibody agent. In some cases, administration of the bryostatin agent is prior to administration of the therapeutically effective amount of an antibody drug conjugate (ADC). In some cases, administration of the bryostatin agent is prior to administration of the therapeutically effective amount of a bispecific antibody agent.

In some embodiments of the methods of treating cancer, the bryostatin agent is administered simultaneously with administration of the therapeutic agent that specifically binds the cell surface antigen to treat the subject for cancer. In some cases, the bryostatin agent is administered simultaneously with administration of the therapeutically effective amount of CAR-T cells. In some cases, the bryostatin agent is administered simultaneously with administration of the therapeutically effective amount of CAR-NK cells. In some cases, the bryostatin agent is administered simultaneously with administration of the therapeutically effective amount of an antibody agent. In some cases, the bryostatin agent is administered simultaneously with administration of the therapeutically effective amount of an antibody drug conjugate (ADC). In some cases, the bryostatin agent is administered simultaneously with administration of the therapeutically effective amount of a bispecific antibody agent.

In some embodiments of the methods of treating cancer, the bryostatin agent is administered subsequently to administration of the therapeutic agent that specifically binds the cell surface antigen to treat the subject for cancer. In some cases, administration of the bryostatin agent is subsequent to administration of the therapeutically effective amount of CAR-T cells. In some cases, administration of the bryostatin agent is subsequent to administration of the therapeutically effective amount of CAR-NK cells. In some cases, administration of the bryostatin agent is subsequent to administration of the therapeutically effective amount of an antibody agent. In some cases, administration of the bryostatin agent is subsequent to administration of the therapeutically effective amount of an antibody drug conjugate (ADC). In some cases, administration of the bryostatin agent is subsequent to administration of the therapeutically effective amount of a bispecific antibody agent. In some cases, administration of bryostatin agent is subsequent to administration of mRNA, enhancing expression, translocation and presentation of the encoded protein.

In some embodiments of the methods of treating cancer, the administration of a therapeutic agent includes administering to the subject a therapeutically effective amount of an antibody agent, ADC, bispecific antibody agent that specifically binds the cell surface antigen.

In some embodiments of the methods of treating cancer, the antibody agent includes a human monoclonal antibody, or antigen-binding portion thereof. In certain cases, the antibody agent is an antibody that comprises a full-length antibody of an IgG1 isotype or an IgG4 isotype.

In some embodiments of the methods of treating cancer, the therapeutic agent that specifically binds the cell surface antigen is an ADC comprising a cytotoxic agent. In certain cases, the cytotoxic agent is a cytotoxin or a radioactive agent. In some cases, the cytotoxic agent is conjugated to an antibody of the ADC via a linker. In certain cases, the linker is selected from peptidyl linkers, hydrazine linkers and disulfide linkers. In certain cases, the cytotoxic agent is selected from calicheamicins, auristatins, maytansinoids, taxol derivatives and duocarmycins.

In some embodiments of the methods of treating cancer, the therapeutic agent that specifically binds the cell surface antigen is an ADC selected from inotuzumab ozogamicin and gemtuzumab ozogamicin.

In some embodiments of the methods of treating cancer, the therapeutic agent that specifically binds the cell surface antigen is a bispecific antibody agent. In certain cases, the bispecific antibody is an anti-CD20/anti-CD22 bispecific antibody fusion protein or an anti-CD19/anti-CD22 bispecific antibody fusion protein.

In some embodiments of the methods of treating cancer, the therapeutic agent is administered via a route selected from orally, ocularly, aurally, subcutaneously, intravenously, intramuscularly, intradermally, intraperitoneally and inhalation. In some cases, the agent is administered orally. In some cases, the agent is administered ocularly. In some cases, the agent is administered aurally. In some cases, the agent is administered subcutaneously. In some cases, the agent is administered intravenously. In some cases, the agent is administered intramuscularly. In some cases, the agent is administered intradermally. In some cases, the agent is administered intraperitoneally. In some cases, the agent is administered by inhalation.

The subject methods can find use in treating a variety of different cancers. For example, representative cancer conditions and cell types against which the methods of the present disclosure may be useful include melanoma, myeloma, chronic lymphocytic leukemia (CLL), AIDS-related lymphoma, non-Hodgkin's lymphoma, colorectal cancer, renal cancer, prostate cancer, cancers of the head, neck, stomach, esophagus, anus, or cervix, ovarian cancer, breast cancer, peritoneal cancer, and non-small cell lung cancer. In one embodiment, the cancer is leukemia or B cell lymphoma. In certain cases, the B cell lymphoma is non-Hodgkin's lymphoma. In one embodiment, the cancer is selected from Burkitt's lymphoma and B cell chronic lymphocytic leukemia. In certain cases, the cancer is melanoma, prostate cancer, breast cancer, ovarian cancer, esophageal cancer, or kidney cancer.

In some embodiments of the methods of treating cancer, the subject is a mammal. In some cases, the mammal is a human. In some cases, the mammal is a non-human, such as, murines (e.g., rats, mice), lagomorphs (e.g., rabbits), non-human primates, canines, felines, ungulates (e.g., equines, bovines, ovines, porcines, caprines), etc.

In some embodiments of the methods of treating cancer, the mammalian subject is relapsed or refractory to cell surface antigen targeted therapy. In certain cases, the cell surface antigen is selected from CD10, CD19, CD20, CD21, CD22, CD27, CD28, CD30, CD33, CD34, CD38, CD40, CD52, CD80, CD86, CD137, CDK4, CDK6, OX40 and CD340.

In some embodiments of the methods of treating cancer, the methods further include determining the level or expression or presentation of the cell surface antigen in target cancer cells of a sample obtained from the subject. In certain cases, cells can be removed from a patient, treated with a bryostatin agent (e.g., as described herein) to enhance expression or surface presentation of a target antigen, then treated with an agent to detect the level of cancer in the patient.

In some embodiments of the methods of treating cancer, the methods further include administering at least one additional anti-cancer therapy to the patient, wherein the additional anti-cancer therapy is selected from radiation therapy, chemotherapy, immunotherapy, checkpoint inhibitors, surgery and vasculature-targeting therapy.

In some embodiments of the methods of treating cancer, the method further includes assessing one or more biomarkers in a sample of the subject to assay the status of the cancer.

In some embodiments of the methods of treating cancer, the bryostatin agent is an analog of bryostatin 1, and the range of tolerated doses of the bryostatin agent is improved by 50% or more, relative to bryostatin 1. In certain cases, the range of tolerated doses of the bryostatin agent is improved by 50% or more, such as 60% or more, 70% or more, 80% or more, 90% or more, 95% or more, or even more, relative to bryostatin 1. In certain cases, the range of tolerated doses of the bryostatin agent is improved by 2-fold or more, such as 3-fold or more, 4-fold or more, 5-fold or more, or even more relative to bryostatin 1.

### Bryostatin Agents

As used herein, the term “bryostatin compound” and “bryostatin agent” are used interchangeably to refer to compounds having an underlying bryostatin pharmacophore, which is based on the bryostatin natural products and in some cases has a scaffold that is characterized by a macrocyclic lactone-containing ring including three embedded six-membered rings (e.g., tetrahydropyran rings designated A, B and C rings), and an arrangement of numbered C1 to C26 carbon atoms, as exemplified in the bryostatin 1 structure shown below. The lactone of the scaffold is defined by a bond between a C1 carbonyl and the oxygen of a C25 hydroxyl group.

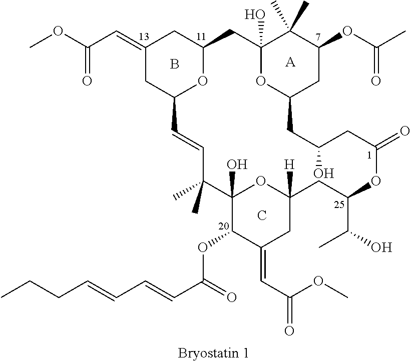

The bryostatin scaffold can include an alkene between C16 and C17 and an exocyclic alkene at positions C13 and C21. The bryostatin scaffold can include a particular arrangement of stereocenters, e.g. at C3, C5, C7, C9, C11, C15, C19, C20 and C23, C25 and/or C26. A variety of substituent groups and derivative groups (e.g., esters or ether groups) can be included in the subject bryostatin compounds (e.g., as described herein). Naturally occurring bryostatins originally isolated from the marine bryozoan include a family of about 21 known compounds. The terms “bryostatin compound” and “bryostatin agent” is meant to include both the naturally occurring bryostatin compounds, such as bryostatin 1, as well as “bryostatin analog compounds”, which include non-naturally occurring bryostatin analogs and derivative compounds of interest that retain functionality required for biological activity.

The terms “bryostatin compound” and “bryostatin agent” is meant to include a compound having the same underlying bryostatin pharmacophore, namely a three dimensional spatial arrangement of three hydrogen bond donors and acceptors that provides its binding function along with a lipid domain that provides for its association with a membrane and for its function derived thereof. This bryostatin pharmacophore, C1 domain model, non-C1 domain target model (e.g., that is modulated by bryostatin or its analogs), or PKC pharmacophore model was introduced by the Wender group in 1986 (see e.g., Wender, PNAS, 1986, 83, 4214-4218), and extended to Bryostatin in 1988 (see e.g., Wender, PNAS, 1988, 85, 7197-7201), leading to the design of the first Bryostatin analogs (e.g., Wender, JACS, 1998, 120, 4534-4535; and Wender, PNAS, 1998, 95, 6624-6629 the disclosures of which are herein incorporated by reference in their entirety). The pharmacophore model is described in U.S. Pat. No. 8,735,609, the disclosure of which is herein incorporated by reference in its entirety. The bryostatin compounds can be broadly described as having two main regions that are referred to herein as a “recognition domain” (or pharmacophoric region) and a relatively lipophilic “spacer domain” (or linker region). The recognition domain contains structural features that are analogous to those spanning C17 through C26 to C1, including the C ring formed in part by atoms C19 through C23, and the lactone linkage between C1 and C25 of the native bryostatin macrocycle. The spacer domain, on the other hand, joins the atoms corresponding to C1 through C17 of the native bryostatin macrocycle to substantially maintain the relative distance between the C1 and C17 atoms and the directionality of the C1C2 and C16C17 bonds. In addition to its function of maintaining the recognition domain in an active conformation, the spacer domain provides a moiety that can be readily derivatized according to any convenient synthetic techniques to provide analogues having improved in vivo stability and pharmacological properties (e.g., by modulating side effect profiles) while retaining biological activity. Exemplary synthetic procedures for obtaining bryostatin 1 and bryostatin analog compounds are described in Wender et al. Science 2017, 358 (6360), 218-223 and International Patent Application No. PCT/US2017/054158, filed Sep. 28, 2017, the disclosures of which are also incorporated herein by reference. The linker region of the bryostatin family can be varied significantly to provide analogs that retain bryostatin-like pan-PKC isoform binding selectivities, binding selectivities for other protein targets with C1 domains, binding selectivities for other non-C1 domain targets, and affinities and other analogs that exhibit selectivities and affinities for only one or more PKC isoforms. Thus, a wide variety of linkers can be used to retain the binding activities of bryostatin 1 or to produce complementary selectivities. Such selectivities influence translocation of PKC, and other target proteins, and its therapeutic activity as well as off target effects. In some cases, the bryostatin compounds include a linker moiety L, which is a linear, cyclic, or polycyclic linker moiety containing a continuous chain of from 6 to 14 chain atoms, one embodiment of which defines the shortest path from C25 via C1 to C17. Distance “d” should be about 2.5 to 5.0 angstroms, preferably about 3.5 to 4.5 angstroms and most preferably about 4.0 angstroms, such as about 3.92 angstroms (as experimentally determined, for example, by NMR spectroscopy). Thus, L may consist solely of a linear chain of atoms that links C17 via C1 to C25, or alternatively, may contain one or more ring structures which help link C17 via C1 to C25. In certain instances, the linker region includes a lactone group (—C(═O)O—), or a lactam group (—C(═O)NH—), which is linked to C25 of the recognition region, by analogy to the C1 lactone moiety that is present in the naturally occurring bryostatins. In addition, the linker can include a hydroxyl group analogous to the C3 hydroxyl found in naturally occurring bryostatins, to permit formation of an intramolecular hydrogen bond between the C3 hydroxyl of the linker and the C19 hydroxyl group of the recognition region (and optionally with the oxygen of the native B ring). In some embodiments, the linker terminates with —CH(OH)CH₂C(═O)O—, for joining to C25 of the recognition region via an ester (or when cyclized, a lactone) linkage. The linker domain is illustrated below:

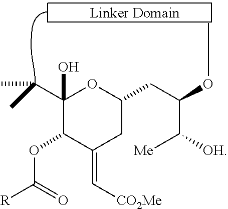

It is understood that for any of the bryostatin compounds described herein, and their synthetic precursors, a numbering scheme can be used to refer to the atoms which correspond to those of the macrocyclic ring and attached substituents as described above for the underlying bryostatin scaffold. Bryostatin compounds of interest include, but are not limited to, any one of the naturally occurring bryostatins, e.g., bryostatin 1, bryostatin 2 and bryostatin 3, and bryostatin analogs, such as those described in U.S. Pat. Nos. 8,735,609, 7,256,286, 8,816,122, 9,096,550, and International Patent Application No. PCT/US2017/054158 the disclosures of which are incorporated by reference herein.

A variety of novel bryostatin compounds are described herein which are accessible via the methods disclosed in International Patent Application No. PCT/US2017/054158. In some cases, the subject bryostatin compounds have activity as protein kinase C modulators both in vitro and in vivo. In some cases, the bryostatin compounds have PKC isoform selectivity. In certain instances, the bryostatin compounds bind to the C1 domain of PKC. In certain instances, the subject bryostatin compounds have activity as a modulator of a signaling protein target that includes a C1 domain. Any convenient C1 domain containing proteins can be targeted for modulation by the subject bryostatin compounds. Exemplary C1 domain containing proteins of interest include, but are not limited to, PKC, PKD, chimaerin, diacylglycerol kinase, Unc-13 and Munc-13, guanine nucleotide exchange factors, myotonic dystrophy kinase-related Cdc42-binding kinase, and the like. Protein targets with “Cl domains” may include any of the following proteins: AKAP13, ARAF, ARHGAP29, ARHGEF2, BRAF, CDC42BPA, CDC42BPB, CDC42BPG, CHN1, CHN2, CIT, DGKA, DGKB, DGKD, DGKE, DGKG, DGKH, DGKI, DGKK, DGKQ, DGKZ, GMIP, HMHA1, KSR1, KSR2, MYO9A, MYO9B, PDZD8, PRKCA, PRKCB1, PRKCD, PRKCE, PRKCG, PRKCH, PRKCI, PRKCN, PRKCQ, PRKCZ, PRKD1, PRKD2, PRKD3, RACGAP1, RAF1, RASGRP, RASGRP1, RASGRP2, RASGRP3, RASGRP4, RASSF1, RASSF5, ROCK1, ROCK2, STAC, STAC2, STAC3, TENC1, UNC13A, UNC13B, UNC13C, VAV1, VAV2, and VAV3. In certain instances, the subject bryostatin compounds have activity as a modulator of a signaling protein target that does not include a C1 domain.

Bryostatin compounds of interest include, but are not limited to, those compounds featuring variation of the C7 ester. Any number of ester or ether substituents can be installed at the C7 position by utilizing a particular anhydride for an esterification reaction, or performing an etherification, e.g., with any number of alkyl bromides or other convenient etherification reagent. In some cases, the subject compounds have A-ring functionalization, e.g., at the C7 and C9 positions that is the same as a target naturally occurring bryostatin, such as bryostatin 1.

In some embodiments, the bryostatin compound has formula (XXIVb):

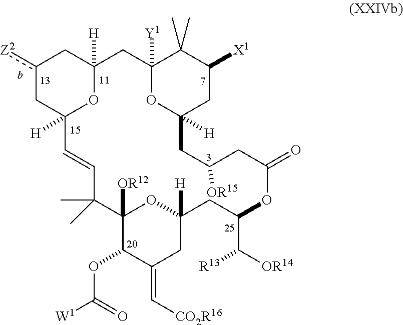

wherein:

W¹ is an alkenyl, a substituted alkenyl, an alkynyl, a substituted alkynyl, an alkyl or a substituted alkyl;
Z² is ═CR⁵R⁶ or ═NR⁷ when the covalent bond designated “b” is a double bond; Z² is —OR⁸ or —N(R⁷)₂ when the covalent bond designated “b” is a single bond;
X¹ is H or OR¹¹;
Y¹ is H or OR¹².
R⁵, R⁶, R⁷ and R⁸ are each independently H, alkyloxycarbonyl (e.g., —CO₂Me), substituted alkyloxycarbonyl, alkyl or substituted alkyl;
R¹¹ is an acyl, a substituted acyl, an alkyl or a substituted alkyl;
each R¹² is independently H, an alkyl or a substituted alkyl;
R¹³ is an alkyl or a substituted alkyl;
R¹⁴ and R¹⁵ are independently H, or a promoiety; and
R¹⁶ is H, an alkyl or a substituted alkyl.

In some embodiments of formula (XXIVb), the bryostatin compound has formula (XXIIb):

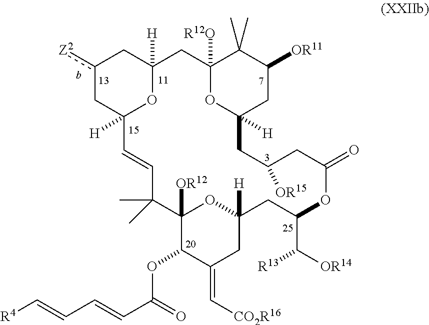

wherein:
R⁴ is an alkyl or a substituted alkyl;
Z² is ═CR⁵R⁶ or ═NR⁷ when the covalent bond designated “b” is a double bond;
Z² is —OR⁸ or —N(R⁷)₂ when the covalent bond designated “b” is a single bond;
R⁵, R⁶, R⁷ and R⁸ are each independently H, alkyloxycarbonyl (e.g., —CO₂Me), substituted alkyloxycarbonyl, alkyl or substituted alkyl;
R¹¹ is H, an acyl, a substituted acyl, an alkyl or a substituted alkyl;
each R¹² is independently H, an alkyl or a substituted alkyl;
R¹³ is H, an alkyl or a substituted alkyl;
R¹⁴ and R¹⁵ are independently H, or a promoiety; and
R¹⁶ is H, an alkyl or a substituted alkyl.

In some embodiments of formula (XXIIb), the bryostatin compound has formula (XXIIIb):

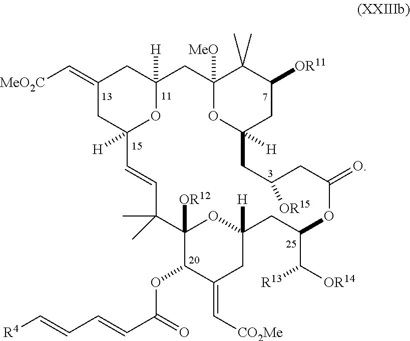

In some instances of the formulae (XXIIb) and (XXIVb), R¹⁶ is methyl. In some instances of the formulae (XXIIb), (XXIIIb) and (XXIVb), R¹⁴ is H or a promoiety. In some instances of the formulae (XXIIb), (XXIIIb) and (XXIVb), R¹³ is methyl. In some instances of the formulae (XXIIb) and (XXIVb), R¹² is methyl. In some instances of the formulae (XXIIb) and (XXIVb), R¹² is H. In some instances of the formulae (XXIIb), (XXIIIb) and (XXIVb), R¹¹ is acetyl. In some instances of the formulae (XXIIb), (XXIIIb) and (XXIVb), R¹¹ is H. In some instances of the formulae (XXIIb) and (XXIIIb), R⁴ is C₃H₇. In some instances of the formulae (XXIIb), (XXIIIb) and (XXIVb), R¹⁵ is H.

In some embodiments, the bryostatin compound is an analog of a naturally occurring bryostatin that has the formula (XXXI):

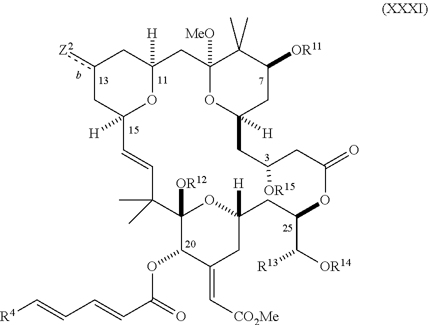

wherein:

R⁴ is an alkyl or a substituted alkyl;

Z² is CR⁵R⁶ or NR⁷ when the covalent bond designated “b” is a double bond;

Z² is OR⁸ or N(R⁷)₂ when the covalent bond designated “b” is a single bond;

R⁵, R⁶, R⁷ and R⁸ are each independently H, halogen, alkyloxycarbonyl, substituted alkyloxycarbonyl, alkyl or substituted alkyl;

R¹¹ is an acyl, a substituted acyl, an alkyl or a substituted alkyl;

R¹² is H, an alkyl or a substituted alkyl;

R¹³ is H, an alkyl or a substituted alkyl; and

R¹⁴ and R¹⁵ are independently H, a hydroxyl protecting group or a promoiety;

or a solvate, hydrate or prodrug form thereof and/or a salt thereof.

In some instances of the formulae (XXXI), R⁴ is propyl. In some instances of the formulae (XXXI), R¹¹ is an alkyl or a substituted alkyl. In some instances of the formulae (XXXI), R¹¹ is an acyl or a substituted acyl. In some instances of the formulae (XXXI), R¹² is an alkyl or a substituted alkyl.

In some instances of the formulae (XXXI), the covalent bond designated “b” is a double bond and Z² is NR⁷ wherein R⁷ is H, alkyloxycarbonyl, substituted alkyloxycarbonyl, alkyl or substituted alkyl. In certain cases, Z² is CFCO₂R′ where R′ is alkyl (e.g., methyl). In some instances of the formulae (XXXI), the covalent bond designated “b” is a single bond and Z² is OR⁸ or N(R⁷)₂, wherein R⁷ and R⁸ are each independently H, alkyloxycarbonyl (e.g., —CO₂Me), substituted alkyloxycarbonyl, alkyl or substituted alkyl.

In some instances, R¹³ is an alkyl comprising at least 2 carbons or a substituted alkyl. In some instances of the formulae (XXXI), R⁴ is a substituted alkyl. In some instances of the formulae (XXXI), R¹⁴ is a promoiety.

It is understood that any of the bryostatin analog compounds, (e.g., as described herein) can be adapted to include an aliphatic (sp3-hybridized) carbon of the main chain (C1-C26) that is substituted with alkyl, substituted alkyl, alkoxy, substituted alkoxy, amino, substituted amino, azido or halogen (e.g., F, Cl) by means of a late-stage (e.g., near the end of the synthesis sequence of steps) C—H oxidation reaction. In some instances, the subject methods further include a late-stage C—H oxidation reaction to install a substituent of interest at one of the C1-C26 positions of the scaffold.

Bryostatin compounds of interest include, but are not limited to, those featuring variation of the C19 hemiketal. Any number of ketals can be installed, e.g., by utilizing any convenient alcohol solvent.

Bryostatin compounds of interest include, but are not limited to, those featuring variation at the C13 position. Variability at the C13 position can be readily accomplished, e.g., through olefination or imine formation. In some cases, the C13 ketone can be reduced to the alcohol and subsequently acylated or etherified. In certain instances, the C13 ketone can be modified via a reductive amination with any convenient amino reactant. In some cases, the E isomer of the C13 enoate can be obtained. In certain instances, the C13 ketone is modified to form a spirocycle. In certain instances, the C13 ketone is modified to form a phosphate, a hetero group, or an organoselenium group.

In some embodiments, the bryostatin compound is an analog of a naturally occurring bryostatin that has the formula (XXXII):

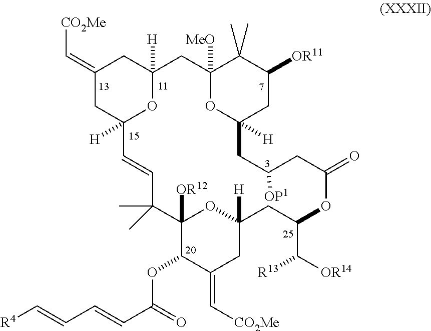

Variability at the C13 position can also be obtained by altering a C13 alkene. In some cases the C13 alkene is substituted, e.g. with an alkyl or a halogen group. In some cases, the C13 alkene can be reduced and optionally further substituted e.g. with an alkyl, alkoxy, halogen group and the like. In some cases the alkene at the C13 position can be modified to form a carbocycle or a heterocycle, e g. through an epoxidation reaction, cyclopropanation reaction, aziridine formation, thiirane formation, cycloadduct formation or spirocycle formation. In some instances, a carbocycle is formed with 3 carbons or more, such as 4 carbons or more, such as 5 carbons or more, such as 6 carbons or more, or even more. In some instances, a three membered heterocycle is formed. In some instances a larger heterocycle is formed, such as a four membered heterocycle, a five membered heterocycle, a six membered heterocycle, or an even larger heterocycle.

In some embodiments, the bryostatin compound is an analog of a naturally occurring bryostatin that is described by any of formulae (XXXIA)-(XXXIC):

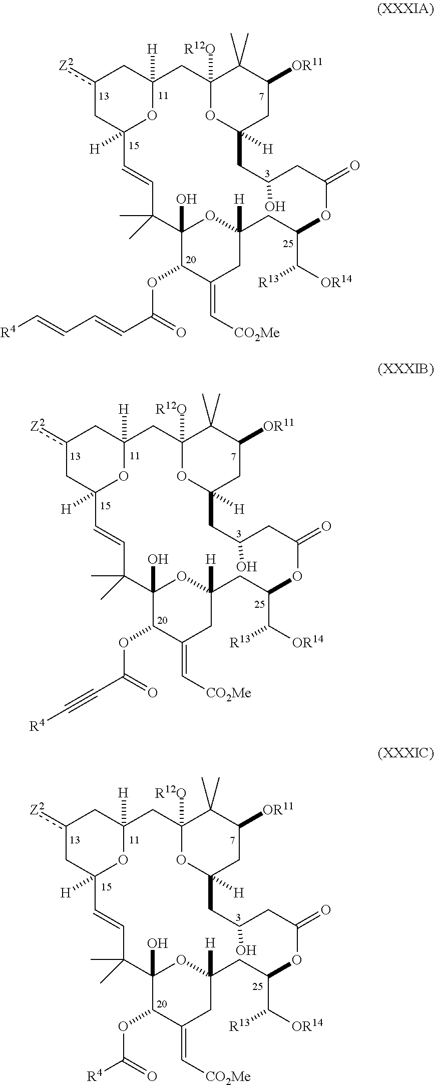

wherein:
R⁴ is an alkyl or a substituted alkyl;
Z² is ═CR⁵R⁶ or ═NR⁷ when the covalent bond designated “b” is a double bond;
Z² is —OR⁸ or —N(R⁷)₂ when the covalent bond designated “b” is a single bond;
R⁵, R⁶, R⁷ and R⁸ are each independently H, alkyloxycarbonyl (e.g., —CO₂Me), substituted alkyloxycarbonyl, alkyl or substituted alkyl;
R¹¹ is H, an acyl, a substituted acyl, an alkyl or a substituted alkyl;
R¹² is H, an alkyl or a substituted alkyl;
R¹³ is H, an alkyl or a substituted alkyl; and
R¹⁴ is H, or a promoiety.

In some instances of any of the formulae (XXXIA)-(XXXIC), R⁴ is selected from propyl, butyl, pentyl, hexyl, heptyl or octyl. In some cases of formula (XXXIA), R⁴ is propyl. In some cases of formula (XXXIB), R⁴ is pentyl. In some cases of formula (XXXIC), R⁴ is heptyl. In some instances of any of the formulae (XXXIA)-(XXXIC), R¹¹ is an alkyl or a substituted alkyl. In some instances of any of the formulae (XXXIA)-(XXXIC), R¹¹ is an acyl or a substituted acyl. In some instances of the formulae (XXXIA)-(XXXIC), R¹² is an alkyl or a substituted alkyl. In some cases, R¹² is hydrogen. In some instances of any of the formulae (XXXIA)-(XXXIC), R¹³ is hydrogen. In some instances of any of the formulae (XXXIA)-(XXXIC), R¹³ is alkyl (e.g., methyl). In some instances of the formulae (XXXIA)-(XXXIC), R¹⁴ is hydrogen.

In some instances of any of the formulae (XXXIA)-(XXXIC), the covalent bond designated “b” is a double bond and Z² is CR⁵R⁶, wherein R⁵, R⁶ are each independently H, alkyloxycarbonyl, or substituted alkyloxycarbonyl. In some cases, R⁵ is H and R⁶ is a substituted alkyloxycarbonyl. In some cases, R⁵ is H and R⁶ is alkyloxycarbonyl (e.g., —CO₂Me or CO₂Et). In some cases both R⁵ and R⁶ are H. In some instances of any one of the formulae (XXXIA)-(XXXIC), the covalent bond designated “b” is a single bond and Z² is OR⁸, wherein R⁸ is selected from H, alkyloxycarbonyl (e.g., —CO₂Me) and substituted alkyloxycarbonyl. In some cases, R⁸ is a substituted alkyloxycarbonyl. In some cases, R⁸ is alkyloxycarbonyl (e.g., —CO₂Me). In some cases R⁸ is H.

In some embodiments of the bryostatin compound is an analog of a naturally occurring bryostatin that is described by any of formulae (XXXIA). In some cases, the structure of formulae (XXXIA) is described by any of the following structures:

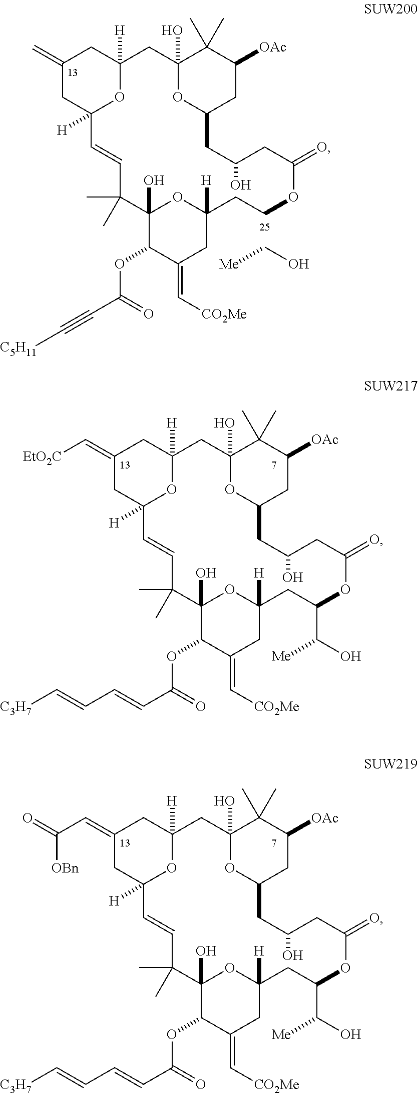

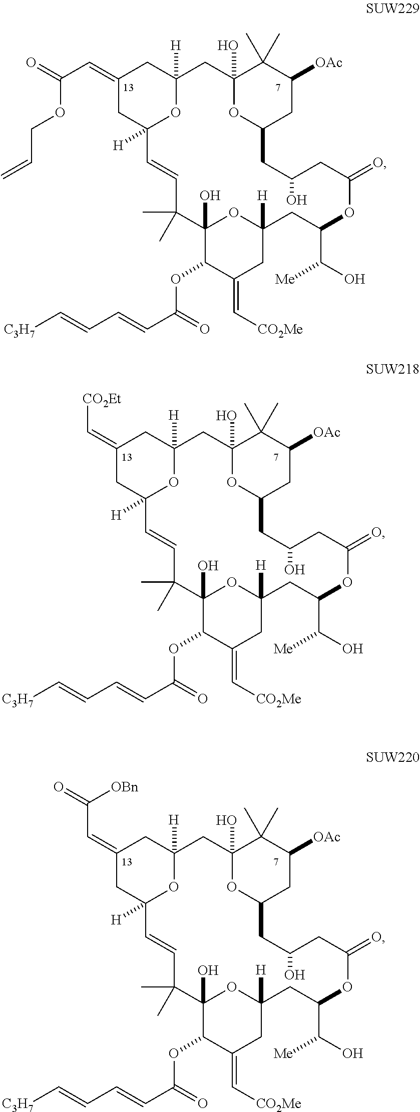

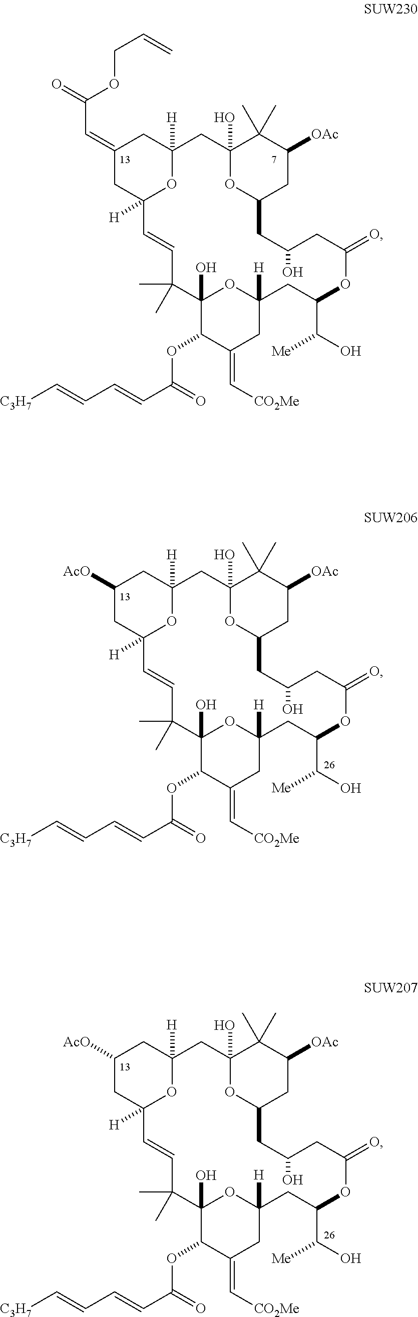

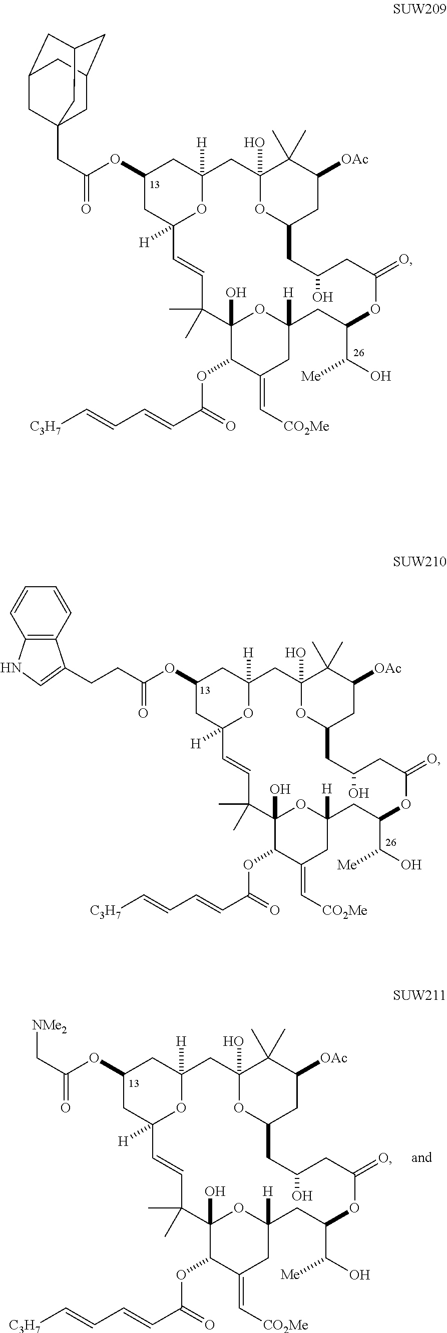

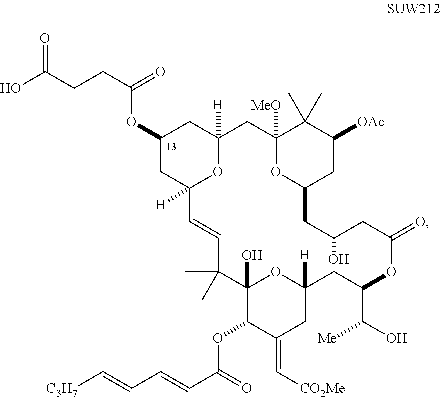

In some embodiments of the bryostatin compound is an analog of a naturally occurring bryostatin that is described by any of formulae (XXXIB). In some cases, the structure of formulae (XXXIB) is described by any of the following structures:

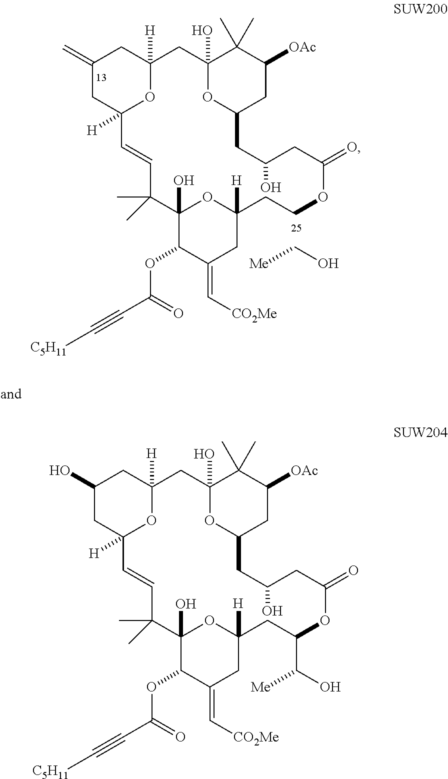

In some embodiments of the bryostatin compound is an analog of a naturally occurring bryostatin that is described by any of formulae (XXXIC). In some cases, the structure of formulae (XXXIC) is described by any of the following structures:

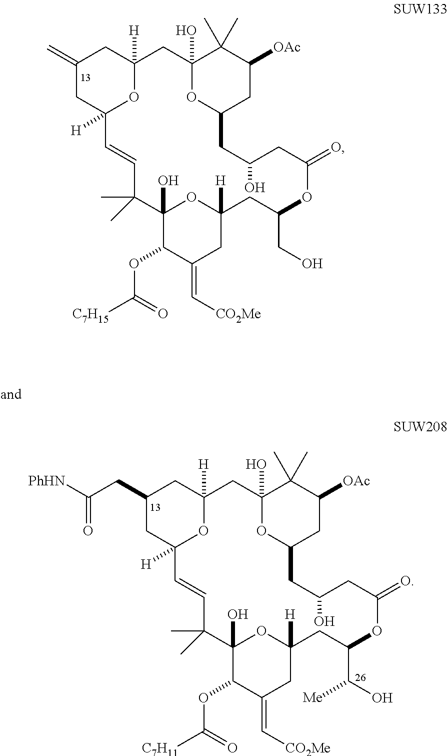

In some embodiments, the bryostatin compound is an analog of a naturally occurring bryostatin that has the formula (XXXIII):

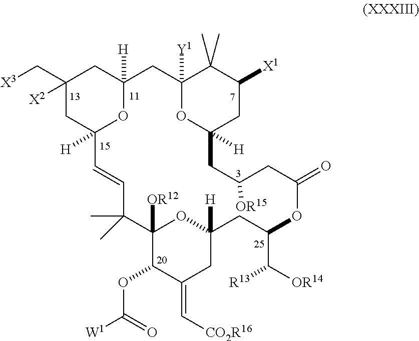

wherein:
W¹ is an alkenyl, a substituted alkenyl, an alkynyl, a substituted alkynyl, an allenyl, a substituted allenyl, an alkyl, a substituted alkyl, an aryl, a substituted aryl, a heteroaryl, a substituted heteoraryl, heteroalkyl, substituted heteroalkyl, heterocycle, substituted heterocycle, or a carbon chain containing oxygen or nitrogen atoms, and/or rings and substituted rings included cyclalkyl, cycloalkenyl and the like (e.g., a PEG or modified PEG group);
X¹ is H or OR¹¹;
X² and X³ are independently selected from H, halogen, alkyl, substituted alkyl, alkoxy, amine, substituted amine, amide, substituted amide, acyl, hydroxyl, heteroalkyl, heteroaryl, substituted hetereoalkyl, substituted heteroaryl, phosphate, organoselenium, thio, substituted thio, or X² and X³ combine to form a carbocyclic ring or a heterocyclic ring e.g. a cyclopropane, an epoxide, an aziridine, a thiirane, a 4-membered spirocycle, a 5-membered spirocycle or a 6 membered spirocycle;
Y¹ is H or OR¹²;
each R¹² is independently H, an alkyl or a substituted alkyl;
R¹³ is H, an alkyl or a substituted alkyl;
R¹⁶ is H, an alkyl or a substituted alkyl; and
R¹⁴ and R¹⁵ are independently H, a hydroxyl protecting group or a promoiety; or a solvate, hydrate or prodrug form thereof and/or a salt thereof.

In certain embodiments of a bryostatin analog of formula (XXXIII), X² and X³ are both hydrogen. In certain cases one of X² or X³ is hydroxyl. In some cases, X² and X³ are both hydroxyl.

In some embodiments of a bryostatin analog of formulae (XXXIII), the compound is described by any of the following structures:

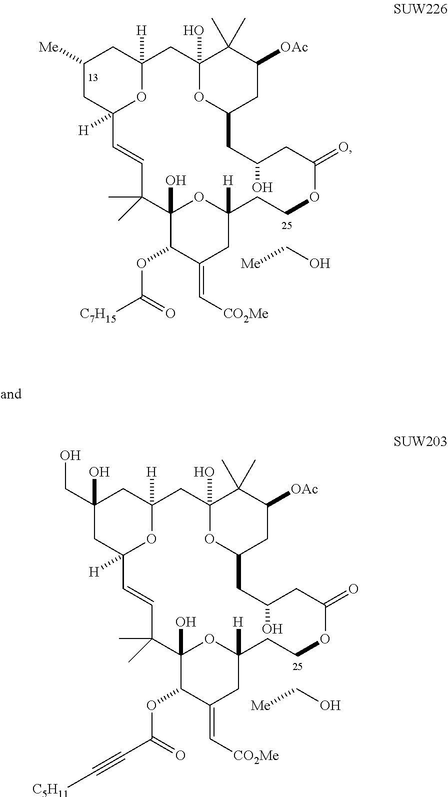

Bryostatin compounds of interest include, but are not limited to, those featuring variation at the C12 or C14 position, or both the C12 and the C14 positions. Variability in the B ring can be obtained by altering the C12 or the C14 carbon, or both the C12 and the C14 carbon in any of the structures and formulae disclosed herein. In some cases the C12 carbon is alkylated. In some cases, the C14 carbon is alkylated. In some cases the C12 carbon is substituted with a halogen. In some cases the C14 carbon is substituted with a halogen. In some cases the C12 or C14 position are independently substituted with a group selected from the group consisting of substituted alkyl, alkoxy, amine, substituted amine, amide, substituted amide, acyl, hydroxyl, heteroalkyl, heteroaryl, substituted hetereoalkyl, substituted heteroaryl, phosphate, phosphoryl, sulfate, sulfonyl, organoselenium, thio, substituted thio.

In some embodiments, the bryostatin compound is an analog of a naturally occurring bryostatin that has the formula (XXXIV):

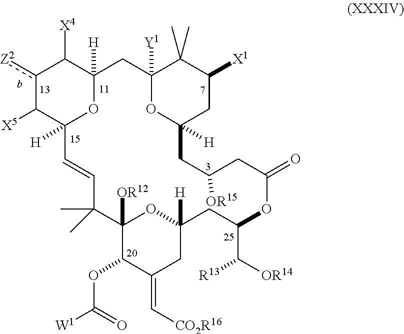

wherein:
W¹ is an alkenyl, a substituted alkenyl, an alkynyl, a substituted alkynyl, an allenyl, a substituted allenyl, an alkyl, a substituted alkyl, an aryl, a substituted aryl, a heteroaryl, a substituted heteroaryl, heteroalkyl, substituted heteroalkyl, heterocycle, substituted heterocycle, or a carbon chain containing oxygen or nitrogen atoms, and/or rings and substituted rings included cycloalkyl, cycloalkenyl and the like (e.g., a PEG or modified PEG group);
Z² is CR⁵R⁶ or NR⁷ when the covalent bond designated “b” is a double bond;
Z² is OR⁸, a phosphate, a phosphoryl, a thio group, a sulfate, a sulfonyl, an organoselenium group, or N(R⁷)₂ when the covalent bond designated “b” is a single bond;
R⁵, R⁶, R⁷ and R⁸ are each independently H, halogen, alkyloxycarbonyl, substituted alkyloxycarbonyl, alkyl or substituted alkyl;
X¹ is OH or OR¹¹;
X⁴ and X⁵ are independently selected from H, halogen, alkyl, substituted alkyl, alkoxy, amine, substituted amine, amide, substituted amide, acyl, hydroxyl, heteroalkyl, heteroaryl, substituted heteroalkyl, substituted heteroaryl, phosphate, organoselenium, thio, substituted thio;
Y¹ is H or OR¹²;
each R¹² is independently H, an alkyl or a substituted alkyl;
R¹³ is H, an alkyl or a substituted alkyl; and
R¹⁶ is H, an alkyl or a substituted alkyl,
R¹⁴ and R¹⁵ are independently H, a hydroxyl protecting group or a promoiety;

or a solvate, hydrate or prodrug form thereof and/or a salt thereof.

Bryostatin compounds of interest include, but are not limited to, those featuring variation at the C26 position. By altering the methods for synthesis of the southern hemisphere fragment, variation at the C26 alcohol can be introduced. In some instances of the subject bryostatin compounds, a hydroxy group at the C26 position is necessary for compound activity. In some cases, variation at the C26 position provides for a prodrug from of a bryostatin compound of interest, where the prodrug form is capable of conversion in vivo to a free C26 hydroxyl group.

In some embodiments, the subject compounds are provided in a prodrug form. “Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)).

Prodrugs of bryostatin compounds include particular bryostatin analogs at C26. The methods of synthesis of the Southern Hemisphere fragment can provide for derivatization of the C26-alcohol, e.g., with an ester. In some instances, introduction of an ester group at the C26 position can inactivate the resulting bryostatin derivative. In certain embodiments, the C26 ester group can be cleaved, e.g., either chemically (e.g., at a particular pH, via photochemical means) or biologically (e.g. via action of an endogenous esterase, reduction of a gamma or epsilon disulfide bond, promoting intramolecular trans thioesterification and release of the free hydroxyl group) to release a bryostatin compound having a free C26 hydroxyl group. This prodrug strategy can provide for facile alteration of a bryostatin compound of interest to improve its pharmacological properties, such as PK (pharmacokinetics) and ADME (absorption, distribution, metabolism, and excretion) properties, while maintaining the activity of bryostatin, which is gradually released as the free drug after compound administration.

The cleavable linkage may include a group that can be hydrolytically, enzymatically, or otherwise cleaved in vivo. The inactive group can range from an alkyl group (e.g., selected to provide a particular cleavage rate) to an oligopeptide or lipid (e.g., to enhance cellular uptake). Modifications can include but are not limited to esters, carbonates, carbamates, and ethers all of which can contain alkyl groups, alkenyl groups, alkynyl groups, amines, hydroxyl groups, guanidinium groups, carbocycles, and heterocycles.

In some embodiments Bryostatin 1 is modified to form a structure of formula (XXXV):

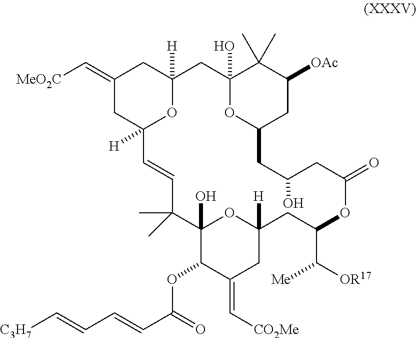

Wherein R¹⁷ is selected from an ester, a carbonate, a carbamate and an ether, all of which can be optionally substituted with one or more groups selected from, an alkyl, an alkenyl, an alkynyl, an amine, a hydroxyl, a disulfide, a guanidinium, a carbocycle, and a heterocycle. In some instances, the acetate group at C7 is replaced with a H atom, an ester, a carbonate, a carbamate or an ether, all of which can be optionally substituted with one or more groups selected from, an alkyl, an alkenyl, an alkynyl, an amine, a hydroxyl, a disulfide, a guanidinium, a carbocycle, and a heterocycle.

Bryostatin compounds of interest include, but are not limited to, those featuring variation at the C26 methyl position. The methods of preparing the southern hemisphere fragment can be adapted to prepare analogs that possess any convenient C26 substituents at the C26 methyl position.

Bryostatin compounds of interest include, but are not limited to, those featuring variation at the C20 ester position. Any convenient ester groups can be installed on the C20 hydroxyl group (e.g., as described herein). In some instances, the ester group is a alkyne containing precursor of an octadienoate group, such as the octadienoate present at the C20 ester position of bryostatin 1. By altering the southern hemisphere synthesis, analogs can be prepared through esterification of a C20 alcohol moiety with a wide variety of ester groups. It is understood that in some cases, any of the ester groups described herein can be referred to as a corresponding acyl or substituted acyl substituent of the C20 hydroxyl. In some cases, the ester group is an alkyl or a substituted alkyl ester. In some cases, the ester group is an aryl or a substituted aryl ester. In some cases, the ester group is an alkenyl or a substituted alkenyl. In some cases, the ester group is an alkynyl or a substituted alkynyl ester.

Bryostatin compounds of interest include, but are not limited to, those featuring variation at the C21 enoate ester position. Any convenient ester groups can be installed on the C20 hydroxyl group (e.g., as described herein).

Aspects of the present disclosure include bryostatin compounds, salts thereof (e.g., pharmaceutically acceptable salts), and/or solvate, hydrate and/or prodrug forms thereof. In addition, it is understood that, in any compound described herein having one or more chiral centers, if an absolute stereochemistry is not expressly indicated, then each center may independently be of R-configuration or S-configuration or a mixture thereof. It will be appreciated that all permutations of salts, solvates, hydrates, prodrugs and stereoisomers are meant to be encompassed by the present disclosure.

In some embodiments, the subject bryostatin compounds, or a prodrug form thereof, are provided in the form of pharmaceutically acceptable salts. Compounds containing an amine, imine or nitrogen containing group may be basic in nature and accordingly may react with any number of inorganic and organic acids to form pharmaceutically acceptable acid addition salts.

In some embodiments, the subject compounds, prodrugs, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

Cell Surface Antigens

As described here, cell surface antigens are meant to include neoantigens and antigens derived from the delivery or expression of mRNA. In certain cases, the cell surface antigen is endogenous. In certain cases, the cell surface antigen is an endogenous neoantigen. In certain cases, the cell surface antigen is derived from administration of mRNA, which is then expressed to produce proteins (e.g., corresponding to the mRNA message) that produce antigens. In this regard, mRNA can be used to express any protein and the externalization of that protein can then create a novel antigen. The protein produced may be any protein that produces antigens, and then the subject bryostatin agents can act to enhance translocation and presentation of the expressed antigen to the cell surface, and extend its persistence on the cell surface.

In some embodiments, the therapeutic agent binds an antigen selected from the group consisting of: 1-40-ű-amyloid, 4-1BB, 5AC, 5T4, activin receptor-like kinase 1, ACVR2B, adenocarcinoma antigen, AGS-22M6, alpha-fetoprotein, angiopoietin 2, angiopoietin 3, anthrax toxin, AOC3 (VAP-1), B7-H3, Bacillus anthracis anthrax, BAFF, beta-amyloid, B-lymphoma cell, C242 antigen, C5, CA-125, Canis lupus familiaris IL31, carbonic anhydrase 9 (CA-IX), cardiac myosin, CCL11 (eotaxin-1), CCR4, CCR5, CD11, CD18, CD125, CD140a, CD147 (basigin), CD15, CD152, CD154 (CD40L), CD19, CD2, CD20, CD200, CD22, CD221, CD23 (IgE receptor), CD25 (Į chain of IL-2receptor), CD27, CD274, CD28, CD3, CD3 epsilon, CD30, CD33, CD37, CD38, CD4, CD40, CD40 ligand, CD41, CD44 v6, CD5, CD51, CD52, CD56, CD6, CD70, CD74, CD79B, CD80, CEA, CEA-related antigen, CFD, ch4D5, CLDN18.2, Clostridium difficile, clumping factor A, CSF1R, CSF2, CTLA-4, C-X-C chemokine receptor type 4, cytomegalovirus, cytomegalovirus glycoprotein B, dabigatran, DLL4, DPP4, DR5, E. coli shiga toxin type-1, E. coli shiga toxin type-2, EGFL7, EGFR, endotoxin, EpCAM, episialin, ERBB3, Escherichia coli, F protein of respiratory syncytial virus, FAP, fibrin II beta chain, fibronectin extra domain-B, folate hydrolase, folate receptor 1, folate receptor alpha, Frizzled receptor, ganglioside GD2, GD2, GD3 ganglioside, glypican 3, GMCSF receptor Į-chain, GPNMB, growth differentiation factor 8, GUCY2C, hemagglutinin, hepatitis B surface antigen, hepatitis B virus, HER1, HER2/neu, HER3, HGF, HHGFR, histone complex, HIV-1, HLA-DR, HNGF, Hsp90, human scatter factor receptor kinase, human TNF, human beta-amyloid, ICAM-1 (CD54), IFN-Į, IFN-Û, IgE, IgE Fc region, IGF-1 receptor, IGF-1, IGHE, IL 17A, IL 17F, IL 20, IL-12, IL-13, IL-17, IL-1ű, IL-22, IL-23, IL-31RA, IL-4, IL-5, IL-6, IL-6 receptor, IL-9, ILGF2, influenza A hemagglutinin, influenza A virus hemagglutinin, insulin-like growth factor I receptor, integrin Į4ű7, integrin Į4, integrin Į5ű1, integrin Į7 ű7, integrin ĮIIbű3, integrin Įvű3, interferon Į/ű receptor, interferon gamma-induced protein, ITGA2, ITGB2 (CD18), KIR2D, Lewis-Y antigen, LFA-1 (CD11a), LINGO-1, lipoteichoic acid, LOXL2, L-selectin (CD62L), LTA, MCP-1, mesothelin, MIF, MS4A1, MSLN, MUC1, mucin CanAg, myelin-associated glycoprotein, myostatin, NCA-90 (granulocyte antigen), neural apoptosis-regulated proteinase 1, NGF, N-glycolylneuraminic acid, NOGO-A, Notch receptor, NRP1, Oryctolagus cuniculus, OX-40, oxLDL, PCSK9, PD-1, PDCD1, PDGF-R Į, phosphate-sodium co-transporter, phosphatidylserine, platelet-derived growth factor receptor beta, prostatic carcinoma cells, Pseudomonas aeruginosa, rabies virus glycoprotein, RANKL, respiratory syncytial virus, RHD, Rhesus factor, RON, RTN4, sclerostin, SDC1, selectin P, SLAMF7, SOST, sphingosine-1-phosphate, Staphylococcus aureus, STEAP1, TAG-72, T-cell receptor, TEM1, tenascin C, TFPI, TGF-ű 1, TGF-ű 2, TGF-ű, TNF-Į, TRAIL-R1, TRAIL-R2, tumor antigen CTAA16.88, tumor specific glycosylation of MUC1, tumor-associated calcium signal transducer 2, TWEAK receptor, TYRP1(glycoprotein 75), VEGFA, VEGFR1, VEGFR2, vimentin, and VWF.

Enhanced CAR-T Cell Therapy Methods

The present disclosure contemplates methods of using CAR-T cell therapy and a bryostatin agent to modulate a T cell-mediated immune response to a target cell population in a subject. The present disclosure also contemplates methods using CAR-NK cell therapy and a bryostatin agent to modulate a NK cell-mediated immune response to a target cell population in a subject. A particular embodiment contemplates a method of modulating a T cell-mediated or NK cell-mediated immune response to a target cell population in a subject, comprising a) introducing to the subject a therapeutically effective plurality of cells genetically modified to express a chimeric antigen receptor, wherein the chimeric antigen receptor comprises at least one antigen-specific targeting region capable of binding to the target cell population, and wherein the binding of the chimeric antigen receptor targeting region to the target cell population is capable of eliciting activation-induced cell death; and b) administering to the subject a therapeutically effective amount of a bryostatin agent sufficient to prevent or limit the activation-induced cell death. In particular embodiments, the CAR comprises an antigen binding domain which specifically recognizes a CD22 target cell population. In certain embodiments of the present disclosure, the bryostatin agent enhances the function of activated memory CD8+ T cells. In other embodiments, the amount of the bryostatin agent administered is sufficient to enhance cytotoxic function. In certain cases, the amount of bryostatin agent administered is sufficient to enhance the activity of CAR T cells by increasing the number of cell surface antigens on target cells.

Embodiments are contemplated wherein administration of the bryostatin agent is prior to, simultaneously with, or subsequent to administration of the therapeutically effective plurality of cells. In certain embodiments of the present disclosure, the bryostatin agent is administered subcutaneously.

In certain cases, the bryostatin agent is contacted with the plurality of cells ex vivo prior to administration to the subject. In certain embodiments, the cells for contacting ex vivo with the bryostatin agent are derived from the subject to be treated (e.g., autologous cells). In other embodiments, the cells for contacting ex vivo with the bryostatin agent are derived from a donor (e.g., allogenic cells).

A chimeric antigen receptor of the present disclosure may include two or more polypeptide chains. In some embodiments, the receptor is a T cell receptor (TCR). In some embodiments, the TCR includes one or more CD3 polypeptides, e.g., one or more CD3ζ polypeptides. In some embodiments, the cell surface receptor is a TCR that includes a protease cleavage site disposed: between the variable region of the alpha chain (αv) and the constant region of the alpha chain (αc); between the constant region of the alpha chain (αc) and the transmembrane region of the alpha chain (αt); between the variable region of the beta chain (βv) and the constant region of the beta chain (βc); between the constant region of the beta chain (βc) and the transmembrane region of the beta chain (βt); if a CD3ζ polypeptide is present, between the transmembrane domain of the CD3ζ polypeptide and the cytoplasmic domain of the CD3ζ polypeptide; or any combination thereof when the TCR includes two or more protease cleavage sites. In some cases, one or more protease cleavage sites (and optionally, one or more corresponding proteases) may be disposed: (1) between the variable region of the alpha chain (αv) and the constant region of the alpha chain (αc); (2) between the constant region of the alpha chain (αc) and the transmembrane region of the alpha chain (αt); (3) between the variable region of the beta chain (βv) and the constant region of the beta chain (βc); (4) between the constant region of the beta chain (βc) and the transmembrane region of the beta chain (βt); and (5) between the transmembrane region of the CD3ζ and the cytoplasmic domain of CD3ζ. In some embodiments, a TCR of the present disclosure includes the cleavage site and the protease (cis configuration), e.g., present within a linker. In some embodiments, when the cell surface receptor is a TCR, the protease is supplied in trans—that is, not part of the polypeptide chain that includes the cleavage site. In some embodiments, when the protease is supplied in trans, the protease is tethered to a different chain of the TCR. For example, when the cleavage site is disposed within the a chain, the protease may be supplied on the R chain, and vice versa. Also by way of example, the cleavage site may be disposed within one of the CD3 chains (epsilon, gamma, delta, or zeta), and the protease may be supplied in a different CD3 chain.

The extracellular binding domain of an engineered receptor of the present disclosure (e.g., a CAR or engineered TCR) may specifically bind to an antigen, e.g., a cell surface antigen, such as an antigen on the surface of a cancer cell, or an antigenic peptide associated with an MHC molecule. The extracellular binding domain “specifically binds” to the antigen if it binds to or associates with the antigen with an affinity or Kₐ (that is, an equilibrium association constant of a particular binding interaction with units of 1/M) of, for example, greater than or equal to about 10⁵ M⁻¹. In certain embodiments, the extracellular binding domain binds to an antigen with a Kₐ greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding refers to binding with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater. Alternatively, affinity may be defined as an equilibrium dissociation constant (K_{D}) of a particular binding interaction with units of M (e.g., 10⁻⁵ M to 10⁻¹³ M, or less). In some embodiments, specific binding means the extracellular binding domain binds to the target molecule with a K_{D} of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the extracellular binding domain for the target antigen can be readily determined using conventional techniques, e.g., by competitive ELISA (enzyme-linked immunosorbent assay), equilibrium dialysis, by using surface plasmon resonance (SPR) technology (e.g., the BIAcore 2000 instrument, using general procedures outlined by the manufacturer); by radioimmunoassay; or the like.

The extracellular binding domain binds to a target antigen of interest, e.g., a particular antigen on the surface of a target cell. An extracellular binding domain may include or consist of an antibody (e.g., a single-chain antibody, such as an scFv), a receptor (e.g., a variable lymphocyte receptor), a receptor fragment (e.g., an Fc receptor fragment), a ligand, a cytokine, a DARPin, an adnectin, a nanobody, and a peptide.

In some embodiments, the extracellular binding domain of the CAR includes a single chain antibody, non-limiting examples of which include a single-chain variable fragment (scFv). The single-chain antibody may be a monoclonal single-chain antibody, a chimeric single-chain antibody, a humanized single-chain antibody, a fully human single-chain antibody, and/or the like (e.g., as described herein). Suitable extracellular binding domains include those described in Labanieh et al. (2018) Nature Biomedical Engineering 2:377-391, the disclosure of which is incorporated herein by reference in its entirety for all purposes. In some embodiments, the extracellular binding domain of the CAR is an antibody approved by the United States Food and Drug Administration and/or the European Medicines Agency (EMA) for use as a therapeutic antibody (e.g., for inducing antibody-dependent cellular cytotoxicity (ADCC) of certain disease-associated cells in a patient, etc.), or a fragment thereof (e.g., a single-chain version of such an antibody, such as an scFv version of the antibody) that retains the ability to bind the target molecule.

In another aspect, the extracellular binding domain of the CAR specifically binds a molecule on the surface of a target cell. The target cell may be any cell type of interest. For example, the target cell may be a genetically and/or phenotypically normal cell. In other embodiments, the target cell is a genetically and/or phenotypically abnormal cell. Abnormal cells of interest include, but are not limited to, cancer cells, cells in the tumor microenvironment (e.g., tumor stromal cells) such as cancer-associated fibroblasts (CAFs), myeloid-derived suppressor cells (MDSCs), tumor-associated macrophages (TAMs), tumor endothelial cells (TECs), and the like. See, e.g., Labanieh et al. (2018) Nature Biomedical Engineering 2:377-391. By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a hematological malignancy (e.g., a leukemia cell, a lymphoma cell, a myeloma cell, etc.), a primary tumor, a metastatic tumor, and the like.

In certain embodiments, the CAR expressing T cells or NK cells are effective for treating B cell malignancy, CLL, ALL, B-ALL, leukemia, lymphoma or solid tumors. In some cases, the solid tumors are selected from breast cancer, prostate cancer, bladder cancer, soft tissue sarcoma, lymphomas, esophageal cancer, uterine cancer, bone cancer, adrenal gland cancer, lung cancer, thyroid cancer, colon cancer, glioma, liver cancer, pancreatic cancer, renal cancer, cervical cancer, testicular cancer, head and neck cancer, ovarian cancer, neuroblastoma and melanoma.

The CAR may include an antigen-binding (e.g., an antibody, such as an scFv), a transmembrane domain, and an intracellular signaling domain.

In some embodiments, the CAR includes one or more linker sequences between the various domains. A “variable region linking sequence” is an amino acid sequence that connects a heavy chain variable region to a light chain variable region and provides a spacer function compatible with interaction of the two sub-binding domains so that the resulting polypeptide retains a specific binding affinity to the same target molecule as an antibody that includes the same light and heavy chain variable regions. In certain aspects, a linker separates one or more heavy or light chain variable domains, hinge domains, transmembrane domains, co-stimulatory domains, and/or primary signaling domains. In particular embodiments, the CAR includes one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

In some embodiments, the binding domain of the CAR is followed by one or more spacer domains that moves the antigen binding domain away from the effector cell surface (e.g., the surface of a T cell expressing the CAR) to enable proper cell/cell contact, antigen binding and/or activation. The spacer domain (and any other spacer domains, linkers, and/or the like described herein) may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain may include the amino acid sequence of a naturally occurring immunoglobulin hinge region or an altered immunoglobulin hinge region. In one embodiment, the spacer domain includes the CH2 and/or CH3 of IgG1, IgG4, or IgD. Illustrative spacer domains suitable for use in the CARs described herein include the hinge region derived from the extracellular regions of type 1 membrane proteins such as CD8a and CD4, which may be wild-type hinge regions from these molecules or variants thereof. In certain aspects, the hinge domain includes a CD8a hinge region. In some embodiments, the hinge is a PD-1 hinge or CD152 hinge.

The “transmembrane domain” (TM domain) is the portion of the CAR that fuses the extracellular binding portion and intracellular signaling domain and anchors the CAR to the plasma membrane of the cell (e.g., immune effector cell). The Tm domain may be derived either from a natural, synthetic, semi-synthetic, or recombinant source. In some embodiments, the Tm domain is derived from (e.g., includes at least the transmembrane region(s) or a functional portion thereof) of the alpha or beta chain of the T-cell receptor, CD35, CD3ζ, CD3γ, CD3δ, CD4, CD5, CD8α, CD9, CD16, CD22, CD27, CD28, CD33, CD37, CD45, CD64, CD80, CD86, CD134, CD137, CD152, CD154, and PD-1.

In one embodiment, a CAR includes a Tm domain derived from CD8a. In certain aspects, a CAR includes a Tm domain derived from CD8a and a short oligo- or polypeptide linker, e.g., between 1, 2, 3, 4, 5, 6, 7, 8, 9, or 10 amino acids in length, that links the Tm domain and the intracellular signaling domain of the CAR. A glycine-serine linker may be employed as such a linker, for example.

The “intracellular signaling” domain of a CAR refers to the part of a CAR that participates in transducing the signal from CAR binding to a target molecule/antigen into the interior of the immune effector cell to elicit effector cell function, e.g., activation, cytokine production, proliferation and/or cytotoxic activity, including the release of cytotoxic factors to the CAR-bound target cell, or other cellular responses elicited with target molecule/antigen binding to the extracellular CAR domain. Accordingly, the term “intracellular signaling domain” refers to the portion of a protein which transduces the effector function signal and that directs the cell to perform a specialized function. To the extent that a truncated portion of an intracellular signaling domain is used, such truncated portion may be used in place of a full-length intracellular signaling domain as long as it transduces the effector function signal. The term intracellular signaling domain is meant to include any truncated portion of an intracellular signaling domain sufficient for transducing effector function signal.

Signals generated through the T cell receptor (TCR) alone are insufficient for full activation of the T cell and a secondary or costimulatory signal is also required. Thus, T cell activation is mediated by two distinct classes of intracellular signaling domains: primary signaling domains that initiate antigen-dependent primary activation through the TCR (e.g., a TCR/CD3 complex) and costimulatory signaling domains that act in an antigen-independent manner to provide a secondary or costimulatory signal. As such, a CAR of the present disclosure may include an intracellular signaling domain that includes one or more “costimulatory signaling domains” and a “primary signaling domain.”

Primary signaling domains regulate primary activation of the TCR complex either in a stimulatory manner, or in an inhibitory manner. Primary signaling domains that act in a stimulatory manner may contain signaling motifs which are known as immunoreceptor tyrosine-based activation motifs (or “ITAMs”). Non-limiting examples of ITAM-containing primary signaling domains suitable for use in a CAR of the present disclosure include those derived from FcRγ, FcRβ, CD3γ, CD3δ, CD3ε, CD3ζ, CD22, CD79α, CD79β, and CD66δ. In certain embodiments, a CAR includes a CD3ζ primary signaling domain and one or more costimulatory signaling domains. The intracellular primary signaling and costimulatory signaling domains are operably linked to the carboxyl terminus of the transmembrane domain.

In some embodiments, the CAR includes one or more costimulatory signaling domains to enhance the efficacy and expansion of T cells expressing the CAR. As used herein, the term “costimulatory signaling domain” or “costimulatory domain” refers to an intracellular signaling domain of a costimulatory molecule or an active fragment thereof. Example costimulatory molecules suitable for use in CARs contemplated in particular embodiments include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, KD2C, SLP76, TRIM, and ZAP70. In some embodiments, the CAR includes one or more costimulatory signaling domains selected from the group consisting of 4-1BB, CD28, CD137, and CD134, and a CD3ζ primary signaling domain.

In certain aspects, a CAR of the present disclosure includes an antigen-binding portion (e.g., a single chain antibody, such as an scFv) that binds to an antigen of interest; a transmembrane domain from a polypeptide selected from the group consisting of: CD4, CD8α, CD154, and PD-1; one or more intracellular costimulatory signaling domains from a polypeptide selected from the group consisting of: 4-1BB, CD28, CD134, and CD137; and an intracellular signaling domain from a polypeptide selected from the group consisting of: FcRγ, FcRβ, CD3γ, CD3δ, CD3ε, CD3ζ, CD22, CD79α, CD79β, and CD66δ. Such a CAR may further include a spacer domain between the antigen-binding portion and the transmembrane domain, e.g., a CD8 alpha hinge.

Enhanced Targeted Anticancer Methods

The present disclosure contemplates methods of enhancing targeted anticancer therapy utilizing a bryostatin agent to modulate target cancer cells to selectively enhance expression or cell surface presentation of an antigen in the cancer cells. A particular embodiment contemplates a method of treating cancer in a subject, comprising a) introducing to the subject a therapeutically effective amount of a bryostatin agent (e.g., as described herein) to enhance cell surface antigen or neoantigen presentation on the target cancer cells; and b) administering to the subject a therapeutically effective amount of therapeutic agent that specifically binds the cell surface antigen to treat the subject for cancer. In particular embodiments, the subject is relapsed or refractory to targeted anticancer therapy.

In certain embodiments of the present disclosure, the target cancer cells prior to treatment with the bryostatin agent present cell surface antigens on the target cell surface at a therapeutically ineffective level. The subject bryostatin agents can enhance the cell surface antigens present at the cell surface, so as to be present at a therapeutically effective level. In some cases, the bryostatin agent enhances expression of cell surface antigens. In some cases, the bryostatin agent enhances translocation of expressed cell surface antigens to the target cell surface. In some cases, the bryostatin agent enhances persistence of cell surface antigens on the target cell surface. Accordingly, the amount of the bryostatin agent administered is sufficient to enhance cytotoxic function of the surface antigens.

In some embodiments of the methods of treating cancer, the bryostatin agent enhances cell surface presentation of the cell surface antigen by 50% or more. In some case, the bryostatin agent enhances cell surface presentation by 55% or more, such as 60% or more, 65% or more, 70% or more, 75% or more, 80% or more, 85% or more, 90% or more, 100% or more, or even more. In some cases, the bryostatin agent enhances cell surface presentation by 2-fold or more, such as 3-fold or more, 4-fold or more, 5-fold or more, or even more.

In some embodiments of the methods of treating cancer, cell surface antigen persistence on the target cancer cell is enhanced for 2 days or more after administration of the bryostatin agent. In certain cases, cell surface antigen presentation on the target cancer cell is enhanced for 3 days or more, such as 4 days or more, 5 days or more, 6 days or more, 7 days or more, 8 days or more, 9 days or more, 10 days or more, or even more after administration of the bryostatin agent.

Embodiments are contemplated wherein administration of the bryostatin agent is prior to, simultaneously with, or subsequent to administration of the therapeutically effective amount of therapeutic agent that specifically binds the cell surface antigen to treat the subject for cancer. In certain embodiments of the present disclosure, the bryostatin agent is administered subcutaneously.

In some embodiments, when the target cell is a cancer cell, the molecule on the surface of the cancer cell to which the antigen-binding portion of the therapeutic agent binds is a tumor-associated cell surface molecule or a tumor-specific cell surface molecule. By “tumor-associated cell surface molecule” is meant a cell surface molecule expressed on malignant cells with limited expression on cells of normal tissues, a cell surface molecule expressed at much higher density on malignant versus normal cells, or a cell surface molecule that is developmentally expressed.

When the target cell is a cancer cell, the cancer cell may express on its surface a tumor-associated molecule or tumor-specific molecule to which the antigen-binding portion of the therapeutic agent binds. In certain embodiments, the target cancer cells comprise a tumor antigen selected from CD10, CD19, CD20, CD21, CD22, CD30, CD34, CD40, CD52, CD80, CD86 and CD340. In certain embodiments, such a tumor-associated molecule or tumor-specific molecule is selected from HER2, B7-H3 (CD276), CD19, CD20, GD2, CD22, CD30, CD33, CD56, CD66/CEACAM5, CD70, CD74, CD79b, CD123, CD133 CD138, CD171, Nectin-4, Mesothelin, Transmembrane glycoprotein NMB (GPNMB), Prostate-Specific Membrane Antigen (PSMA), SLC44A4, CA6, tyrosine-protein kinase Met (c-Met), epidermal growth factor receptor variant III (EGFRvIII), mucin 1 (MUC1), ephrin type-A receptor 2 (EphA2), glypican 2 (GPC2), glypican 3 (GPC3), fms-like tyrosine kinase 3 (FLT3), folate receptor alpha (FRα), IL-13 receptor alpha 2 (IL13Ra2), fibroblast activation protein (FAP), receptor tyrosine kinase-like orphan receptor 1 (ROR1), B-cell maturation antigen (BCMA), delta-like 3 (DLL3), κ light chain, vascular endothelial growth factor receptor 2 (VEGFR2), Trophoblast glycoprotein (TPBG), anaplastic lymphoma kinase (ALK), CA-IX, an integrin, C-X-C chemokine receptor type 4 (CXCR4), neuropilin-1 (NRP1), matriptase, and any other tumor-associated or tumor-specific molecules of interest.

In certain embodiments, the therapeutic agent that specifically binds the cell surface antigen to treat the subject for cancer is an antibody agent. Antibodies that can be used as inhibitors in connection with the present disclosure can encompass, but are not limited to, monoclonal antibodies, polyclonal antibodies, bispecific antibodies, Fab antibody fragments, F(ab)₂ antibody fragments, Fv antibody fragments (e.g., V_{H} or V_{L}), single chain Fv antibody fragments and dsFv antibody fragments. Furthermore, the antibody molecules can be fully human antibodies, humanized antibodies, or chimeric antibodies. The antibodies that can be used in connection with the present disclosure can include any antibody variable region, mature or unprocessed, linked to any immunoglobulin constant region. Minor variations in the amino acid sequences of antibodies or immunoglobulin molecules are encompassed by the present disclosure, providing that the variations in the amino acid sequence maintain 75% or more, e.g., 80% or more, 90% or more, 95% or more, or 99% or more of the sequence. In particular, conservative amino acid replacements are contemplated. Conservative replacements are those that take place within a family of amino acids that are related in their side chains. Whether an amino acid change results in a functional peptide can be determined by assaying the specific activity of the polypeptide derivative.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment which contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

Antibodies that can be used in connection with the present disclosure thus can encompass monoclonal antibodies, polyclonal antibodies, bispecific antibodies, Fab antibody fragments, F(ab)2 antibody fragments, Fv antibody fragments (e.g., VH or VL), single chain Fv antibody fragments and dsFv antibody fragments. Furthermore, the antibody molecules can be fully human antibodies, humanized antibodies, or chimeric antibodies. In some embodiments, the antibody molecules are monoclonal, fully human antibodies.

The antibodies that can be used in connection with the present disclosure can include any antibody variable region, mature or unprocessed, linked to any immunoglobulin constant region. If a light chain variable region is linked to a constant region, it can be a kappa chain constant region. If a heavy chain variable region is linked to a constant region, it can be a human gamma 1, gamma 2, gamma 3 or gamma 4 constant region, more preferably, gamma 1, gamma 2 or gamma 4 and even more preferably gamma 1 or gamma 4.

Minor variations in the amino acid sequences of antibodies or immunoglobulin molecules are encompassed by the present invention, providing that the variations in the amino acid sequence maintain at least 75%, e.g., at least 80%, 90%, 95%, or 99% of the sequence. In particular, conservative amino acid replacements are contemplated. Conservative replacements are those that take place within a family of amino acids that are related in their side chains. Whether an amino acid change results in a functional peptide can readily be determined by assaying the specific activity of the polypeptide derivative. Fragments (or analogs) of antibodies or immunoglobulin molecules, can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Sequence motifs and structural conformations can be used to define structural and functional domains in accordance with the invention.

Non-limiting examples of antibodies which may find use of the present disclosure include Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or an antigen-binding variant thereof. As used herein, the term “variant” refers to an antibody that binds to a particular cognate antigen (e.g., HER2 for trastuzumab) but has fewer or more amino acids than the parental antibody, has one or more amino acid substitutions relative to the parental antibody, is a single-chain variant (such as an scFv variant) of the parental antibody, or any combination thereof.

In certain embodiments, the therapeutic agent that specifically binds the cell surface antigen to treat the subject for cancer is an antibody (e.g., as described herein) conjugated to a cytotoxic agent. In certain embodiments the cytotoxic agent is a cytotoxin or a radioactive agent. In certain cases, the cytotoxic agent is selected from calicheamicins, auristatins, maytansinoids, taxol derivatives and duocarmycins.

In certain cases, the cytotoxic agent is chemotherapeutic agent. Specific chemotherapeutic agents of interest include, but are not limited to, Gemcitabine, Docetaxel, Bleomycin, Erlotinib, Gefitinib, Lapatinib, Imatinib, Dasatinib, Nilotinib, Bosutinib, Crizotinib, Ceritinib, Trametinib, Bevacizumab, Sunitinib, Sorafenib, Trastuzumab, Ado-trastuzumab emtansine, Rituximab, Ipilimumab, Rapamycin, Temsirolimus, Everolimus, Methotrexate, Doxorubicin, Abraxane, Folfirinox, Cisplatin, Carboplatin, 5-fluorouracil, Teysumo, Paclitaxel, Prednisone, Levothyroxine, Pemetrexed, navitoclax, and ABT-199. Peptidic compounds can also be used. Cancer chemotherapeutic agents of interest include, but are not limited to, dolastatin and active analogs and derivatives thereof; and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); benzodiazepines and active analogs and derivatives thereof (e.g., pyrrolobenzodiazepine (PBD); calicheamicins and active analogs and derivatives thereof (e.g., antibody-drug conjugates using calicheamicins include gemtuzumab ozogamicin, and inotuzumab ozogamicin). In some embodiments, the ADC is selected from inotuzumab ozogamicin and gemtuzumab ozogamicin.

In certain embodiments of the ADC, the cytotoxic agent is conjugated to the antibody via a linker. Any convenient linking groups can be utilized in the subject ADCs. The terms “linker”, “linkage” and “linking group” are used interchangeably and refer to a linking moiety that covalently connects two or more compounds (e.g., an antibody to a cytotoxic agent of interest). In some cases, the linker is divalent. In certain cases, the linker is a branched or trivalent linking group. In some cases, the linker has a linear or branched backbone of 200 atoms or less (such as 100 atoms or less, 80 atoms or less, 60 atoms or less, 50 atoms or less, 40 atoms or less, 30 atoms or less, or even 20 atoms or less) in length. A linking moiety may be a covalent bond that connects two groups or a linear or branched chain of between 1 and 200 atoms in length, for example of about 1, 2, 3, 4, 5, 6, 8, 10, 12, 14, 16, 18, 20, 30, 40, 50, 100, 150 or 200 carbon atoms in length, where the linker may be linear, branched, cyclic or a single atom. In certain cases, one, two, three, four or five or more carbon atoms of a linker backbone may be optionally substituted with a sulfur, nitrogen or oxygen heteroatom. In certain instances, when the linker includes a PEG group, every third atom of that segment of the linker backbone is substituted with an oxygen. The bonds between backbone atoms may be saturated or unsaturated, usually not more than one, two, or three unsaturated bonds will be present in a linker backbone. The linker may include one or more substituent groups, for example an alkyl, aryl or alkenyl group. A linker may include, without limitations, oligo(ethylene glycol), ethers, thioethers, disulfide, amides, carbonates, carbamates, tertiary amines, alkyls, which may be straight or branched, e.g., methyl, ethyl, n-propyl, 1-methylethyl (iso-propyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable. A linker may be peptidic, e.g., a linking sequence of residues. A linker may be a hydrazine linker. A linker may be a disulfide linker.

In certain embodiments, the therapeutic agent that specifically binds the cell surface antigen to treat the subject for cancer is a bispecific antibody. In certain cases, the bispecific antibody is an anti-CD20/anti-CD22 bispecific antibody fusion protein. In certain cases, the bispecific antibody is an anti-CD19/anti-CD22 bispecific antibody fusion protein.

In certain embodiments of the enhanced targeted anticancer methods, the subjects innate immune system is effective to act as the therapeutic agent to treat the subject for the cancer. In certain cases of the enhanced targeted anticancer methods, the subjects adaptive immune system is effective to act as the therapeutic agent to treat the subject for cancer. In this regard, a particular embodiment contemplates a method of treating cancer in a subject, comprising a) introducing to the subject a therapeutically effective amount of a bryostatin agent (e.g., as described herein) to enhance cell surface antigen or neoantigen presentation on the target cancer cells; and b) clearance of the enhanced cell surface antigen or neoantigens by the subject's immune system to treat the subject for cancer.

Compositions

Aspects of the invention also include compositions, e.g., compositions comprising a bryostatin agent of interest.

The herein-discussed compositions can be formulated using any convenient excipients, reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the bryostatin agent is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

In some embodiments, the bryostatin agent and a second therapeutic agent that specifically binds the cell surface antigen (e.g., as described herein), e.g. chimeric antigen receptor expressing T cells (CAR T-cells), antibody agent, antibody drug conjugate (ADC) and bispecific antibody agent, etc. (e.g., as described herein) are administered to individuals in a formulation (e.g., in the same or in separate formulations) with a pharmaceutically acceptable excipient(s). In some embodiments, the formulation is conjointly administering at least one additional anti-cancer therapy to the patient, wherein the additional anti-cancer therapy is selected from radiation therapy, chemotherapy, immunotherapy, checkpoint inhibitors, surgery and vasculature-targeting therapy. In certain embodiments, the checkpoint inhibitor is selected from a cytotoxic T-lymphocyte-associated antigen 4 (CTLA-4) inhibitor, a programmed death 1 (PD-1) inhibitor, or a PD-L1 inhibitor.

In another aspect, a pharmaceutical composition is provided, comprising, or consisting essentially of, a bryostatin agent, or a pharmaceutically acceptable salt, isomer, tautomer or prodrug thereof, and further comprising one or more additional anti-cancer agents of interest. Any convenient anti-cancer agents can be utilized in the subject methods in conjunction with the subject compounds. The subject compounds may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

Although such may not be necessary, compounds and agents described herein can optionally be targeted to the site of cancer, using any known targeting means. The compounds of the disclosure may be formulated with a wide variety of compounds that have been demonstrated to target compounds to the site of cancer. The terms “targeting to the site of cancer” and “cancer targeted” refer to targeting of a compound to a site of cancer, such that at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%, or more, of the compound administered to the subject enters the site of cancer.

### Dosage and Administration

In some embodiments, a “therapeutically effective amount” is an amount of a subject bryostatin agent that, when administered to an individual in one or more doses, in monotherapy or in combination therapy, is effective to enhance expression of a protein in the target cells in the subject by at least about 20%, at least about 30%, at least about 40%, at least about 50%, at least about 60%, at least about 70%, at least about 80%, or at least about 90%, compared to the expression of the protein in the target cells in the absence of treatment with the bryostatin agent. In other embodiments, a “therapeutically effective amount” is an amount of a subject bryostatin agent that, when administered to an individual in one or more doses, in monotherapy or in combination therapy, is effective to enhance surface presentation of a protein in the target cells in the subject by at least about 20%, at least about 30%, at least about 40%, at least about 50%, at least about 60%, at least about 70%, at least about 80%, or at least about 90%, compared to the surface presentation of the protein in the target cells in the absence of treatment with the bryostatin agent.

In some embodiments, an effective amount of a bryostatin agent is an amount that ranges from about 50 ng/ml to about 50 μg/ml (e.g., from about 50 ng/ml to about 40 μg/ml, from about 30 ng/ml to about 20 μg/ml, from about 50 ng/ml to about 10 μg/ml, from about 50 ng/ml to about 1 μg/ml, from about 50 ng/ml to about 800 ng/ml, from about 50 ng/ml to about 700 ng/ml, from about 50 ng/ml to about 600 ng/ml, from about 50 ng/ml to about 500 ng/ml, from about 50 ng/ml to about 400 ng/ml, from about 60 ng/ml to about 400 ng/ml, from about 70 ng/ml to about 300 ng/ml, from about 60 ng/ml to about 100 ng/ml, from about 65 ng/ml to about 85 ng/ml, from about 70 ng/ml to about 90 ng/ml, from about 200 ng/ml to about 900 ng/ml, from about 200 ng/ml to about 800 ng/ml, from about 200 ng/ml to about 700 ng/ml, from about 200 ng/ml to about 600 ng/ml, from about 200 ng/ml to about 500 ng/ml, from about 200 ng/ml to about 400 ng/ml, or from about 200 ng/ml to about 300 ng/ml).

In some embodiments, an effective amount of a bryostatin agent is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 μg, from about 1 μg to about 10 μg, from about 10 μg to about 50 μg, from about 50 μg to about 150 μg, from about 150 μg to about 250 μg, from about 250 μg to about 500 μg, from about 500 μg to about 750 μg, from about 750 μg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

In some embodiments, a single dose of a bryostatin agent is administered. In other embodiments, multiple doses are administered. Where multiple doses are administered over a period of time, the compound can be administered twice daily (bid), daily (qd), every other day (qod), every third day, three times per week (tiw), or twice per week (biw) over a period of time. For example, a compound is administered bid, qd, qod, tiw, or biw over a period of from one day to about 2 years or more. For example, a bryostatin agent is administered at any of the aforementioned frequencies for one week, two weeks, one month, two months, six months, one year, or two years, or more, depending on various factors. In some embodiments, the compound may be administered orally, ocularly, aurally, subcutaneously, intravenously, intramuscularly, intradermally, intraperitoneally and by inhalation, among other routes of administration. In some embodiments, the compound may be administered in courses wherein “drug holidays” are allowed that may last from 1-7 days.

In certain embodiments, the dose of a bryostatin agent is a prodrug of a bryostatin agent, e.g., as described herein.

Kits

Also provided are kits that include bryostatin agents of the present disclosure. Kits of the present disclosure may include one or more dosages of the bryostatin agent, and optionally one or more dosages of one or more additional therapeutic agents. In some embodiments the kit includes a one or more dosages of a bryostatin agent (e.g., as described herein); and one or more dosages of a therapeutically effective amount of a therapeutic agent that specifically binds the cell surface antigen (e.g., as described herein). Conveniently, the formulations may be provided in a unit dosage format. In such kits, in addition to the containers containing the formulation(s), e.g. unit doses, is an informational package insert describing the use of the subject formulations in the methods of the invention, e.g., instructions for using the subject unit doses to treat cellular conditions associated with pathogenic angiogenesis. The term kit refers to a packaged active agent or agents. In some embodiments, the subject system or kit includes a dose of a subject compound (e.g., as described herein) and a dose of a second active agent (e.g., as described herein) in amounts effective to treat a subject for a disease or condition associated with angiogenesis (e.g., as described herein).

In addition to the above-mentioned components, a subject kit may further include instructions for using the components of the kit, e.g., to practice the subject method. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, DVD-ROM, BluRay, diskette, Hard Disk Drive (HDD), portable flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In some embodiments, a kit includes a first dosage of a subject pharmaceutical composition comprising a bryostatin agent and a second dosage of a subject pharmaceutical composition comprising a therapeutic agent of interest.

Utility

The subject methods find use in selectively enhancing the expression or cell surface presentation of a protein in target cells of interest to modulate activity of the target cells. The subject methods may find use in a variety of applications, including therapeutic, diagnostic and research applications, in which the modulation of cells of interest is desirable.

The subject methods may find use in the treatment of diseases for which there are no effective therapies, including the eradication of HIV/AIDS and cancer. The subject methods may also find use in sensitizing target cells of interest to clearance by innate or adaptive immune system cells. The subject methods may also find use in treating a subject who is relapsed or refractory to targeted anticancer therapy.

The subject methods may find use in diagnostic applications, including assessing one or more biomarkers in a sample of a subject to assay the status of a disease (e.g., cancer).

The following example(s) is/are offered by way of illustration and not by way of limitation.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Introduction

The development of targeted biologics and cell therapies for the treatment of cancer, including monoclonal antibodies (mAbs), antibody-drug conjugates (ADC's), bi-specific antibodies (biAbs) and chimeric antigen receptor (CAR) T cells, is revolutionizing oncology. By targeting tumor-specific cell surface antigens and neoantigens, these therapies can offer distinct advantages over traditional treatment options as they can avoid the systemic toxicity associated with cytotoxic chemotherapies while efficiently and selectively clearing malignant cells. While mAbs, ADCs, biAbs, and CAR T therapies rely on vastly different mechanisms of action and leverage different host biological systems for tumor clearance, each is fundamentally based on a common requirement, specifically sufficient and sustained target antigen presentation. Notwithstanding the clinical promise of these recent advances in immuno-oncology, tumor escape and acquired resistance driven by decreased surface expression of target antigens limit the efficacy and scope of these approaches. Indeed, poor durability of response and patient relapse associated with variable and decreased antigen expression have been observed across several indications. See, e.g., Lim et al. Cell 2017, 168(4), 724-740; June et al. Science 2018, 359 (6382), 1361-1365; Sharma et al. Cell 2017, 168 (4), 707-723; Loganzo et al. Mol. Cancer Ther. 2016, 15 (12), 2825-2834; Fry et al. Nat. Med. 2017; and Majzner et al. Cancer Discov. 2018, 8 (10), 1219-1226. The identification of adjuvants that enhance and sustain surface expression of target antigens could broadly address this problem and improve the efficacy of these approaches, as they could be used in combination with targeted biologics and cell therapies to increase both the number of patient responders and the durability of the response.

Bryostatin 1, a marine macrolide and potent PKC modulator (see. e.g., Pettit et al. J. Am. Chem. Soc. 1982, 104 (24), 6846-6848; and Kortmansky et al. Cancer Invest. 2003, 21 (6), 924-936), can alter expression of surface antigens in tumor and other cell lines, making them more immunogenic and thus more susceptible to immune clearance. Several pre-clinical and clinical studies have reported that bryostatin 1 can alter the immunophenotype and increase the immunogenicity of cancer cells in acute lymphoblastic leukemia (ALL), chronic lymphocytic leukemia (CLL), and non-Hodgkin's lymphoma (NHL) (see e.g., Hammond et al. J. Immunother. 2005, 28 (1), 28-39; Katib et al. Hematol. 1993, 21 (1), 61-64; Varterasian et al. J. Clin. Oncol. 1998, 16 (1), 56-62; Al Katib et al. J. Immunother. 1993, 14, 33-42; Varterasian et al. Clin. Cancer Res. 2000, 6 (3), 825-828; and Shaha et al. Clin. Exp. Immunol. 2009, 158 (2), 186-198). It has also been demonstrated that a designed bryostatin analog (bryology or “bryostatin agent”), like bryostatin 1, can increase cell surface antigen presentation on chronic lymphocytic leukemia (CLL) cells (Hammond et al. J. Immunother. 2005, 28 (1), 28-39; and Katib et al. J. Immunother. 1993, 14, 33-42).

Additionally, it has also been found that bryostatin 1 and some of its analogs can induce the presentation of CD69, a cell surface activation marker on latently HIV infected CD4+ T cells, thus serving as preclinical leads in a “kick and kill” approach to HIV eradication (DeChristopher et al. Nat. Chem. 2012, 4 (9), 705-710; Marsden et al. PLOS Pathog. 2017, 13 (9), e1006575; and Albert et al. Sci. Rep. 2017, 7 (1), 7456).

Among antigen targeting strategies in clinical evaluation, CAR T cell therapy has emerged in recent years as a highly effective treatment for B-cell malignancies. Despite this, antigen loss has been observed as a primary driver in acquired resistance and patient relapse. The present inventors discovered that bryostatin 1 and exemplary analogs can be used in combination with anti-CD22 CAR T therapy to improve patient outcomes by ensuring that malignant cells maintain sufficient levels of CD22 surface expression to be effectively cleared.

The vast majority of research in this area has focused on only a small number of natural products and more specifically bryostatin 1. As evident from studies on the taxanes and avermectins, immediate synthetic precursors and derivatives of natural products, so called “close-in” analogs, often provide superior clinical performance (Omura et al. Nat. Rev. Microbiol. 2004, 2 (12), 984-989; and Pazdur et al. Cancer Tret. Rev. 1993, 19 (4), 351-386). Unfortunately, access to such analogs in the case of bryostatin 1 has been precluded by the lack of available material required to make synthetic derivatives, which is further exacerbated by the inherent challenges associated with modifying the exquisitely complex bryostatin scaffold. The original “hand collection” of bryostatin, which provided material for clinical use, produced only 18 grams of material from 14 tons of the source organism Bugula neritina (0.00014% yield) and is now nearly depleted (Schaufelberger et al. J. Nat. Prod. 1991, 54 (5), 1265-1270). Re-collection from this marine source would raise environmental concerns due to bryostatin's poor and variable natural availability (Keough, J. Biol. Bull. 1989, 177 (2), 277-286). Acquaculture (“in tank” and “in sea”) and engineered biosynthesis have been explored, but the former encountered capitalization and yield problems and the latter difficulties in cultivation of the symbiotic bacterium necessary for production of bryostatin 1 (Mendola, D. Biomol. Eng. 2003, 20 (4-6), 441-458. Recently, the inventors reported a solution to this problem, a practical chemical synthesis of bryostatin 1, that has afforded for the first-time sustainable access to gram scale quantities of the natural product as needed to insure its continued clinical evaluation (Wender et al. Science 2017, 358 (6360), 218-223). Additionally, the chemical synthesis can also serve as a platform for the development of bryostatin analogs and the exploration of structure activity relationships at various positions around bryostatin's macrocyclic skeleton, thereby enabling the design and synthesis of the next generation of bryostatin-inspired small molecules for research and the treatment of human disease. Given that bryostatin 1 is in preclinical and clinical studies for numerous indications including the treatment of Alzheimer's disease, eradication of HIV/AIDS, multiple sclerosis, Niemann Pick disease, Fragile X, and enhanced immunotherapy, and given that many of these indications involve different PKC isoforms, it is proposed that different analogs might exhibit superior activity across different indications due to their differing selectivity's and tolerability's.

### Example 1—Design of Bryostatin Analogs

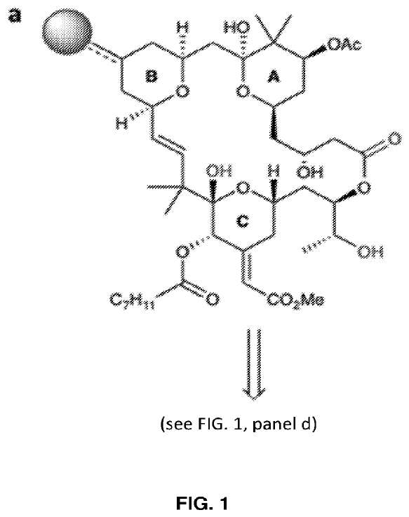

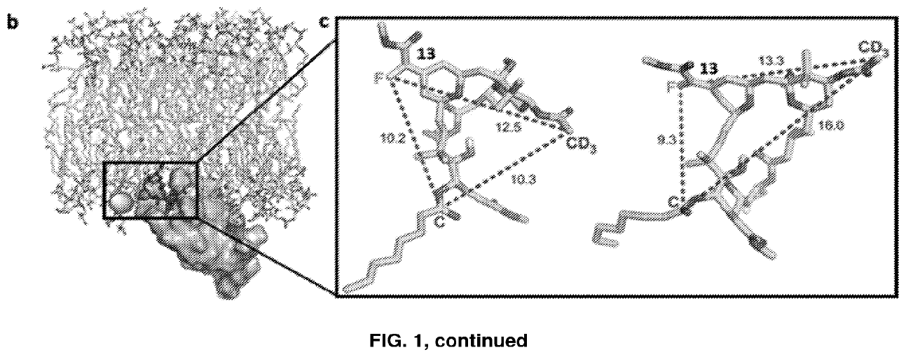

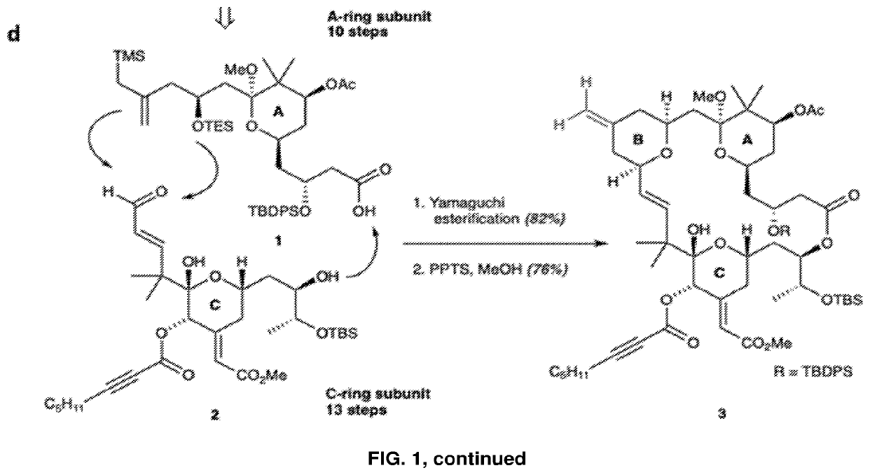

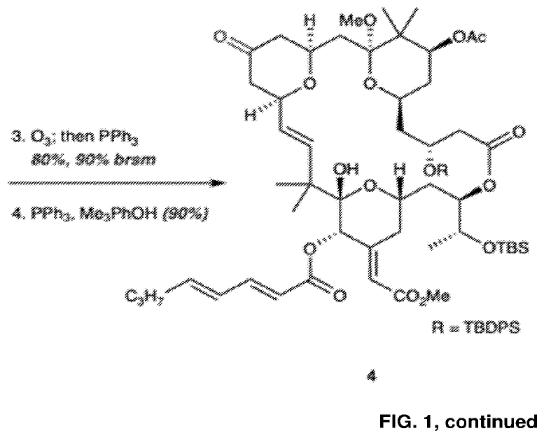

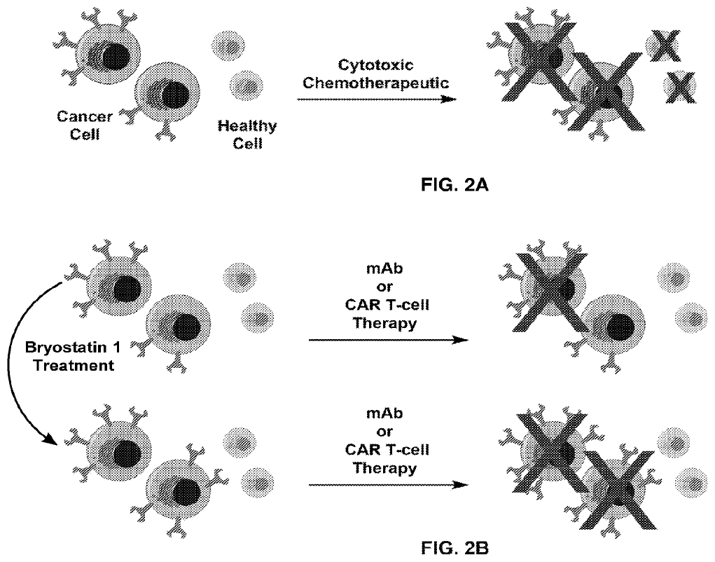

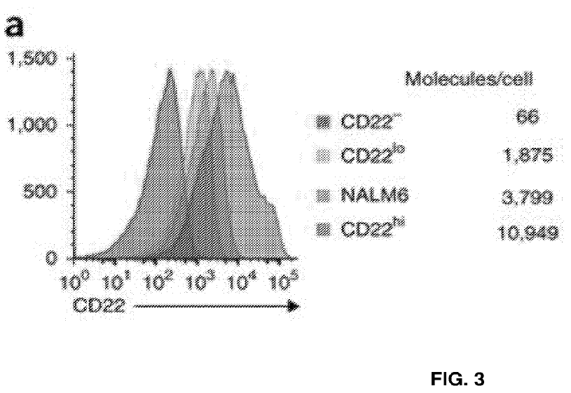

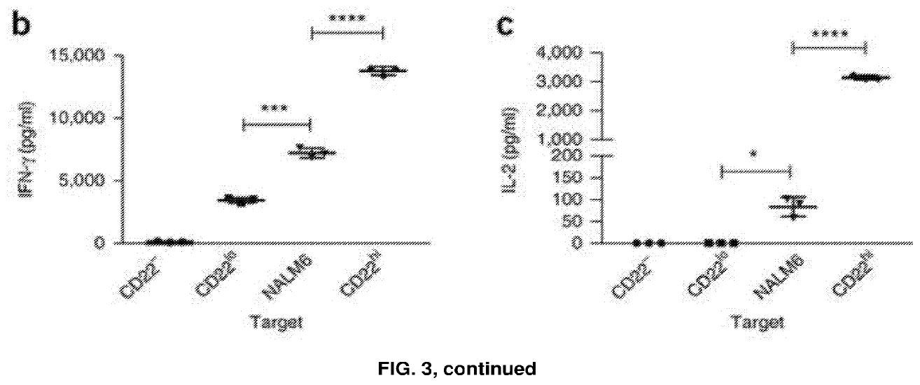

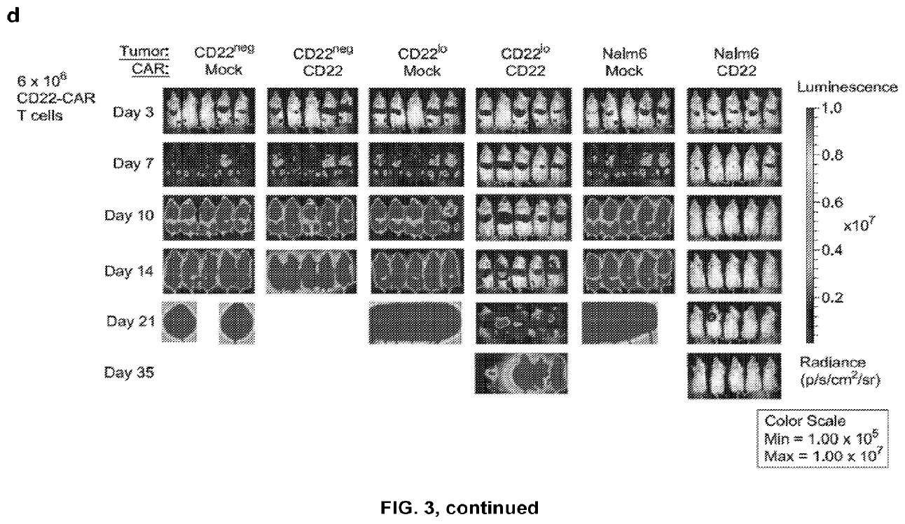

Herein is disclosed the design, synthesis, and evaluation of exemplary bryostatin analogs. Design strategy focused on making chemical modifications to the bryostatin scaffold that would not be expected to significantly impact compound affinity to PKC but could influence PKC function and potentially be used as needed to tune formulation and ADME (absorption, distribution, metabolism, and excretion) characteristics (FIG. 1, panel A). 18 analogs were prepared.

FIG. 1. (Panel A) Retrosynthetic analysis of the bryostatin scaffold with pharmacophoric elements of the C-ring subunit identified to be the C1 carbonyl, C19 hemiketal, and C26 alcohol. C13 (indicated by sphere) highlighted as an area of interest for analog synthesis. (Panel B) Rendering of the PKC-bryostatin-membrane ternary complex. Bryostatin 1 shown in the rectangular box. Pharmacophoric elements of the C-ring subunit of the bryostatin scaffold (see FIG. 1, panel A) interact directly with PKC (dark gray) while the A and B rings are imbedded in the plasma membrane (light gray). See ACS Cent. Sci. 2018, 4, 89-96. (Panel C) Representative conformers of the bryostatin scaffold bound to PKC that fit experimentally determined intramolecular distances determined by REDOR NMR. See ACS Cent. Sci. 2018, 4, 89-96. (Panel D) Convergent construction of the bryostatin scaffold from acid 1 and enal 2. C13 functionality provides a versatile starting point for late-stage diversification, avoiding interference with the pharmacophoric elements of the C ring (as described above for FIG. 1, panel A).

These compounds were designed to retain the pharmacophoric functionalities proposed for PKC binding in our original pharmacophore model. Consistent with that model, most of the exemplary analogs disclosed herein exhibited single digit nanomolar affinities to representative PKC isoforms, comparable to bryostatin 1 (Wender et al. Proc. Natl. Acad. Sci. U.S.A. 1986, 83 (12), 4214-4218; and Wender et al. Proc. Natl. Acad. Sci. U.S.A. 1988, 85 (19), 7197-7201). In contrast, a diverse array of activity profiles were observed in a functional assay measuring PKC activation in living cells, suggesting that the modifications made can indeed elicit differential biological functions, irrespective of cell-free binding affinity data. Significantly, in connection with antigen-targeted therapies, the ability of exemplary analogs to increase CD22 surface expression in an in vitro model of ALL was also investigated. It was found that several exemplary analogs exhibited activity similar to bryostatin 1, suggesting that these compounds could serve as more accessible, efficacious, and better tolerated adjuvants for cancer immunotherapy and other indications.

Design strategy for exemplary bryostatin analogs was guided by our previously proposed bryostatin pharmacophore model (Wender et al. Proc. Natl. Acad. Sci. U.S.A. 1988, 85 (19), 7197-7201) and further augmented by molecular dynamics simulations (Ryckbosch et al. Nat. Commun. 2017, 8 (1), 6) and REDOR NMR studies (Yang et al. ACS Cent. Sci. 2018, 4 (1), 89-96) of bryostatin 1 and a labeled analog bound to PKC in a membrane microenvironment. PKC is a family of seven homologous signaling kinases classified as conventional (α, β, γ) or novel (δ, ε, η, θ) based on their subdomain architectures (Newton, A. C. J. Biol. Chem. 1995, 270 (48), 28495-28498). Individual isoforms, combinations of isoforms, and mutant isoforms of PKC are implicated in a number of disease pathologies (Newton, A. C. AJP Endocrinol. Metab. 2010, 298 (3), E395-E402; and Newton, A. C. Semin. Cancer Biol. 2018, 48, 18-26). PKC maturation and activation are governed by a sequence of highly orchestrated phosphorylations, conformational changes, and ultimately translocation to the plasma membrane where they effect phosphorylations of numerous downstream signaling proteins (Newton, A. C. J. Biol. Chem. 1995, 270 (48), 28495-28498; and Newton, A. C. Chem. Rev. 2001, 101 (8), 2353-2364). The hallmark of ligand-induced PKC activation is formation of a ternary complex between the plasma membrane, ligand, and PKC (FIG. 1, panel B). In this context, the bryostatin scaffold can be considered as two subunits that have distinct but interdependent functions. Our original computational similarity search across a number of PKC modulators revealed that hydrogen-bonding functionalities in or proximate to the C-ring subunit of the bryostatin scaffold, specifically the C1 carbonyl, C26 hydroxyl, and C19 hemiketal, are spatially preorganized by the A and B rings of the northern subunit into a binding conformation that mimics PKC's endogenous ligand, DAG (FIG. 1, panels A and C) (Wender et al. Proc. Natl. Acad. Sci. U.S.A. 1988, 85 (19), 7197-7201; Ryckbosch et al. Nat. Commun. 2017, 8 (1), 6; Yang et al. ACS Cent. Sci. 2018, 4 (1), 89-96; and Wender et al. Proc. Natl. Acad. Sci. U.S.A. 1998, 95 (12), 6624-6629). Indeed, seminal studies indicated that modification or deletion of C1, C26 and C19 functionalities reduced or eliminated PKC binding while changes in the A- and B-rings generally had little or no effect on binding, suggesting that changes to these subunits in exemplary bryostatin analogs could be used to change function and biodistribution (Wender et al. Proc. Natl. Acad. Sci. U.S.A 1998, 95 (12), 6624-6629; Wender et al. Proc. Natl. Acad. Sci. U.S.A. 1988, 85 (19), 7197-7201; Wender et al. In Natural Products in Medicinal Chemistry; Wiley-VCH, 2014; pp 473-544; Wender et al. J. Am. Chem. Soc. 2008, 130 (21), 6658-6659; Wender et al. Proc. Natl. Acad. Sci. U.S.A. 2011, 108 (17), 6721-6726; and DeChristopher et al. Nat. Chem. 2012, 4 (9), 705-710).

In addition to spatially restricting the pharmacophoric elements of bryostatin's C-ring subunit to a productive PKC-binding conformation, the northern subunit is also thought to influence translocation efficiency of PKC as well as the depth and orientation of the ligand-PKC complex in membranes, as suggested by recent long-timescale (400-500 μs) molecular dynamics simulations (Wender et al. Proc. Natl. Acad. Sci. U.S.A. 1988, 85 (19), 7197-7201; Ryckbosch et al. Nat. Commun. 2017, 8 (1), 6; Yang et al. ACS Cent. Sci. 2018, 4 (1), 89-96; and Wender et al. Proc. Natl. Acad. Sci. U.S.A. 1998, 95 (12), 6624-6629). Specifically, interaction of oxygenation in the A and B-rings with water molecules at the membrane-cytosol interface putatively causes the bryostatin-PKC complex to adopt a shallow, angled orientation with respect to the membrane (FIG. 1, panel B) (Ryckbosch et al. Nat. Commun. 2017, 8 (1), 6; Yang et al. ACS Cent. Sci. 2018, 4 (1), 89-96). This orientation was found to be unique to the bryostatin 1-PKC complex and was not populated when other potent PKC ligands such as phorbol dibutyrate (PDBu) and prostratin were modeled, providing a possible explanation for their competitive PKC binding but often contrasting downstream activities. In a separate study, a combination of REDOR NMR and molecular dynamics simulations identified a distribution of PKC-bound bryostatin conformers (FIG. 1, panel C), a feature that was unique to the bryostatin scaffold and not observed in a phorbol ester derivative (Yang et al. ACS Cent. Sci. 2018, 4 (1), 89-96). Together, these studies suggest that the multiple PKC-bound conformers available to the bryostatin scaffold could generate differential orientations of the activated PKC-ligand complex and influence PKC's interactions with downstream effector proteins, thereby explaining bryostatin's unique biological activity. This in turn suggests that modifications to the A and B rings of the bryostatin scaffold could influence compound function by affecting the conformational energy landscape of the bryostatin scaffold and ultimately the orientation of the active PKC signaling complex. Using this guiding framework, a series of compounds were designed that can retain the pharmacophoric elements of the bryostatin C-ring but incorporate a series of systematic modifications to the bryostatin B-ring at C13. This site was selected because computational studies suggested that modifications at this position would preserve PKC binding affinity but could influence function. By leveraging the unique reactivity at this position found in late stage synthetic intermediates exemplary bryostatin analogs could be generated with diverse chemical functionality at this position to explore how various substitution patterns in the northern hemisphere can affect compound function by influencing membrane association, trafficking, and downstream signaling outcomes.

### Example 2—Synthesis of Bryostatin Analogs

Previously the inventors reported a convergent, step-economical synthesis of bryostatin 1, which allowed for the assembly of a diversifiable bryostatin macrocylic skeleton (exo-olefin 3, FIG. 1, panel D) in 25 total steps and 15 steps in the longest linear sequence from two building blocks of roughly equal complexity (FIG. 1, panel D), see, e.g., Wender et al. Science 2017, 358 (6360), 218-223; and International Patent Application No. PCT/US2017/054158, filed Sep. 28, 2017 the disclosures of which are incorporated herein by reference. With reference to FIG. 1, panel D, Yamaguchi esterification brings carboxylic acid 1 and enal 2 together in 82% yield. Subsequent Prins-driven macrocylization forms bryostatin's B-ring and closes the macrocycle. Importantly, the Prins annulation generates exo-olefin 3, which was expected to serve as a chemical handle for late-stage diversification of this advanced intermediate. Exo-olefin 3 can be selectively modified to directly install new B ring functionality or converted into the corresponding ketone 4 via a stoichiometric ozonolysis procedure developed by our group. These two functional groups afford chemical reactivity that is largely orthogonal to the functionality that decorates the remainder of the complex and delicate bryostatin scaffold. As such, we sought to use these groups as starting points for exploring how modifications to bryostatin's B-ring can influence compound function.

During initial exploration of the chemical space at C13, it was sought to leverage the unique reactivity of the C13 exo-olefin to generate a series of exemplary structural analogs. Previous synthetic work on the bryostatin scaffold indicated that this olefin is likely to be the most reactive of the four pi systems found in this advanced intermediate (Wender et al. Science 2017, 358 (6360), 218-223). An exemplary synthetic scheme for the chemoselective functionalization of the C13 exo-olefin is shown below (Scheme 1).

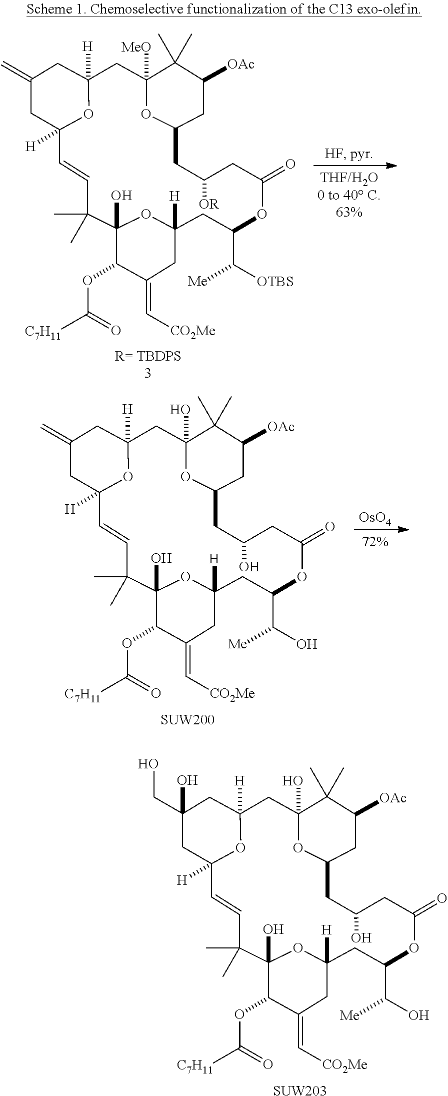

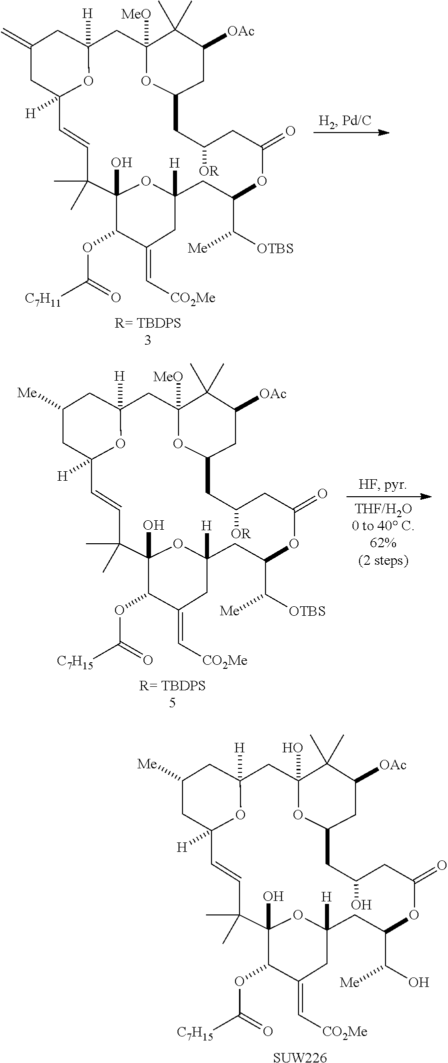

With reference to Scheme 1, di-hydroxylation of SUW200 cleanly afforded vicinal diol 5 in the presence of the C16/C17 alkene, C20 alkynoate and C21 dienoate. In contrast, epoxidations of the exo-olefin 3 and bryostatin 1 with DMDO and mCPBA proved challenging to control, but interestingly provided different chemoseletivities. Attempts to reduce the C13 olefin using H₂ and catalytic Pd/C were also difficult to control, often providing mixtures of reduction products. However, we were able to achieve clean reduction of the C13 exo-olefin and C20 alkynoate without reducing either the C21 enoate or the C16/17 olefin to generate compound 5, which was subsequently deprotected to afford SUW226. Additionally, olefin 3 can be directly deprotected as previously described to afford SUW200 (Wender et al. Science 2017, 358 (6360), 218-223).

Following efforts to modify the C13 olefin, next attention was focused on modification of the C13 ketone 4. This advanced intermediate served as an ideal diversification point as the C13 ketone presents chemical reactivity that is orthogonal to the rest of the functionality found in this series of advanced intermediates, thus circumventing problems arising from the selective modification of the C13 exo-olefin in 3 in the presence of several other pi-systems. An exemplary synthetic scheme for the chemoselective functionalization of the C13 ketone 4 is shown below (Scheme 2).

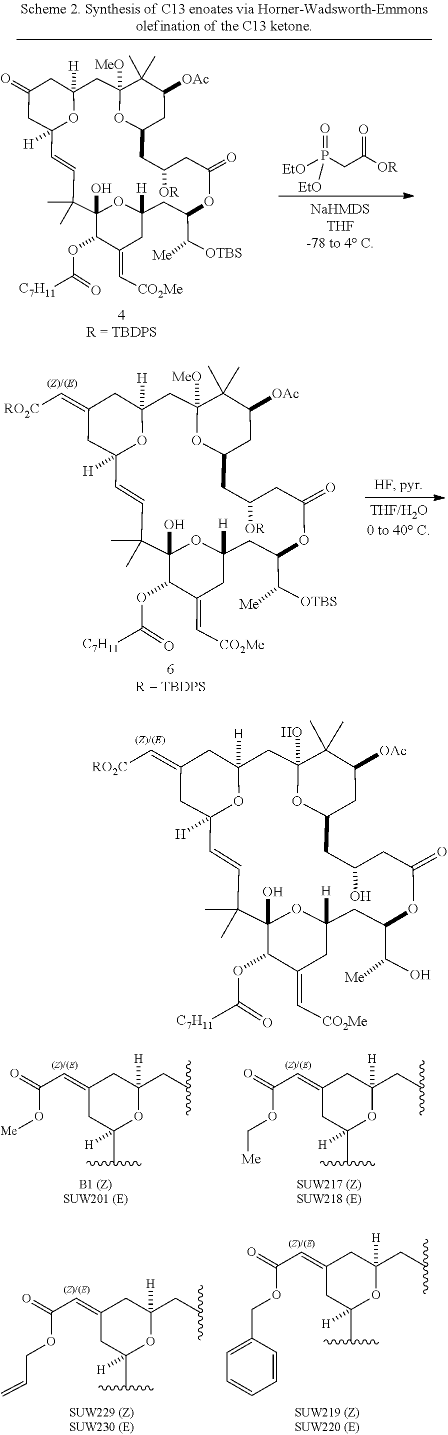

With reference to Scheme 2, the synthesis began with the synthesis of a series of alkyl enoates at the C13 position. From ketone 4, a series of compounds were generated with various ester groups to explore how substituent size and enoate geometry affected compound function. Ester derivatives were accessed via a Horner-Wadsworth-Emmons (HWE) olefination of ketone 4 using the corresponding HWE reagents that were prepared via straightforward coupling of the desired alcohol to diethyl phosphonoacetic acid. Contrasting the use of a chiral HWE reagent to control double bond geometry reported in our bryostatin 1 synthesis, we chose to perform these reactions using simple, non-stereoselective HWE reagents in order to access both the (E) and (Z) isomers of the olefination product 6. Subsequent global deprotection and HPLC separation of the enoate diastereomers afforded exemplary analogs.

In addition to ketone-derived unsaturated esters, installation of an alcohol at C13 was expected to provide a convenient functional handle for the late stage diversification through esterification and an opportunity to explore how C13 hydridization affects function. This was readily accomplished via borohydride reduction of ketone 4. With the C13 alcohol in hand, a number of ester groups were installed via standard esterification procedures (Scheme 3), giving rise to a structurally diverse set of compounds, which included the C13 alcohol, aliphatic esters, an indoyl ester, a phenyl carbamate, and both cationic and anionic moieties.

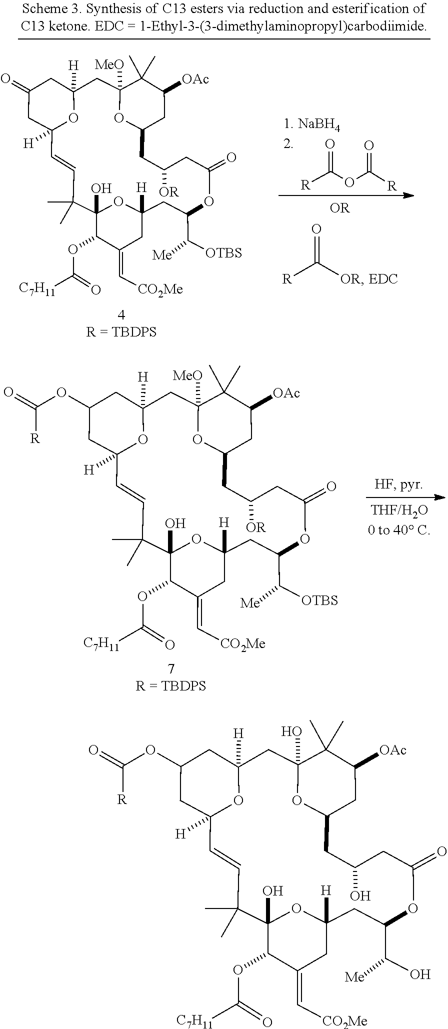

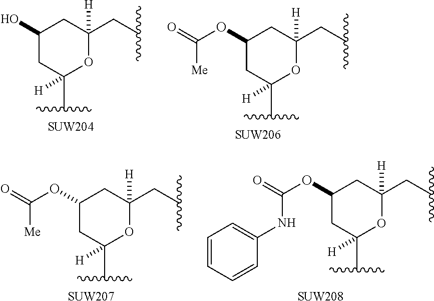

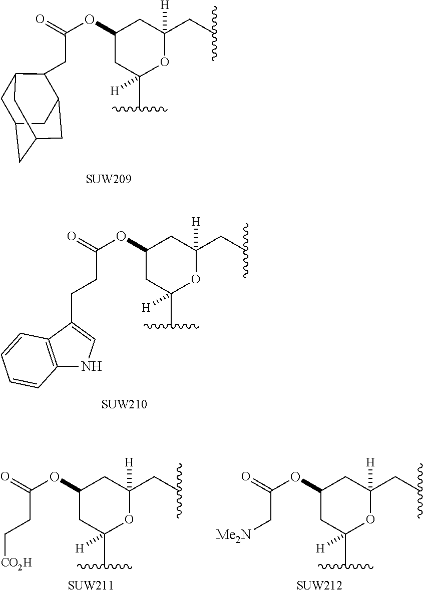

### Synthetic Procedures

Compounds may be synthesized using any convenient method. Methods which can be adapted for use in preparing compounds of this disclosure includes the exemplary synthetic methods described in Wender et al. Science 2017, 358 (6360), 218-223, and International Patent Application No. PCT/US2017/054158, filed Sep. 28, 2017. Many general references providing commonly known chemical synthetic schemes and conditions useful for synthesizing the disclosed compounds are also available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978). Reactions may be monitored by thin layer chromatography (TLC), LC/MS and reaction products characterized by LC/MS and ¹H NMR. Intermediates and final products may be purified by silica gel chromatography or by HPLC.

All reactions were conducted in oven- or flame-dried glassware under a nitrogen or argon atmosphere unless otherwise noted. Reactions were concentrated under reduced pressure with a rotary evaporator unless otherwise noted. Commercial reagents were used as received or purified using the methods indicated herein. Dichloromethane, diethyl ether, dimethylformamide, pentane, tetrahydrofuran, and toluene were passed through an alumina-drying column (Solv-Tek Inc.) using nitrogen pressure; ethyl acetate, hexanes, and petroleum ether were obtained from Fisher Scientific. Analytical thin-layer chromatography (TLC) was carried out on 250 m silica gel 60G plates with fluorescent indicator F254 (EMD Millipore). Plates were visualized with UV light and treated with p-anisaldehyde, ceric ammonium molybdate, or potassium permanganate stain with gentle heating. Flash column chromatography was performed using silica gel (230-400 mesh, grade 60, particle size 40 to 63 m) purchased from Fischer Scientific. pH 7 buffered silica gel was prepared by adding 10% weight pH 7 phosphate buffer to silica and rotating for ˜12 hrs. NMR spectra were acquired on a Varian INOVA 600, Varian INOVA 500, or Varian 400 magnetic resonance spectrometer. ¹H chemical shifts are reported relative to the residual solvent peak (CHCl₃=7.26 ppm, C₆H₆=7.16 ppm) as follows: chemical shift (6), multiplicity (app=apparent, b=broad, s=singlet, d=doublet, t=triplet, q=quartet, m=multiplet, or combinations thereof), coupling constant(s) in Hz, integration. ¹³C chemical shifts are reported relative to the residual solvent peak (CHCl₃=77.16 ppm, C₆H₆=128.06 ppm). Infrared spectra were acquired on a Nicolet iS 5 FT-IR Spectrometer (ThermoFisher). Optical rotations were acquired on a P-2000 Digital Polarimeter (Jasco). High-resolution mass spectra (HRMS) were acquired at the Vincent Coates Foundation Mass Spectrometry Laboratory at Stanford.

### Synthetic Procedure for SUW200

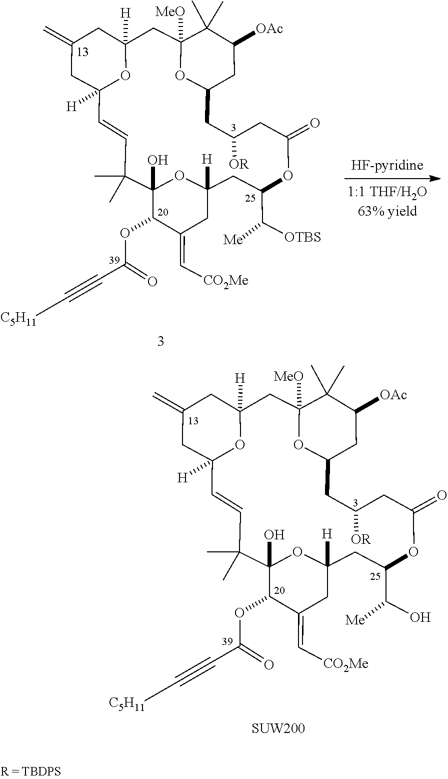

To a 15 mL polypropylene falcon tube equipped with magnetic stir bar was added compound 3 (15 mg, 0.0124 mmol, 1 equiv) and 3:1 THF/H₂O (1 mL). The falcon tube was transferred to a 4° C. cold room. HF-pyridine (0.32 mL) was added (final concentration ˜0.01M). After 96 h, the reaction mixture was warmed to room temperature. After an additional 64 h (˜6.5 days in total), the reaction mixture was quenched by slowly syringing the solution into a separatory funnel containing saturated aqueous NaHCO₃ (20 mL) and EtOAc (20 mL). The layers were separated, and the aqueous layer was extracted with EtOAc (4×20 mL). The combined organic layers were washed with 0.5M HCl (10 mL) to remove pyridine, and aqueous layer back-extracted with EtOAc (2×20 mL). The combined organic layers were dried over Na2SO₄, filtered, and concentrated. Purification was accomplished by silica gel flash column chromatography (25-65% EtOAc/Hex) affording SUW200 (6.6 mg, 63% yield) as a white solid. Compound purity was established by TLC (one spot) analysis. TLC R_{f}=0.56 (60% EtOAc/Hex, UV active, dark purple spot in p-anisaldehyde); [α]^{22.3}_{D}=−1.8° (c=0.18, CH₂Cl₂); IR (thin film): 3460, 3331, 2929, 2855, 2234, 1738, 1716, 1666, 1435, 1408, 1366, 1244, 1157, 1098, 1055, 982 cm⁻¹; ¹H-NMR (600 MHz, CDCl₃) δ 5.98 (d, J=2.0 Hz, 1H, C₃₄H), 5.77 (d, J=15.8 Hz, 1H, C₁₇H), 5.30 (dd, J=15.8, 8.6 Hz, 1H, C₁₆H), 5.28 (s, 1H, C₁₉—OH), 5.21 (ddd, J=12.0, 5.5, 3.0 Hz, 1H, C₂₅H), 5.16 (dd, J=11.9, 4.8 Hz, 1H, C₇H), 5.14 (s, 1H, C₂₀H), 4.76 (bs, 1H, C₃₀Hₐ), 4.74 (bs, 1H, C₃₀H_{b}), 4.27 (d, J=12.1 Hz, 1H, C₃—OH), 4.27-4.19 (m, 1H), 4.20-4.13 (m, 1H), 4.07-3.99 (m, 2H), 3.85-3.76 (m, 1H), 3.72-3.67 (m, 1H, C₂₂Hₐ), 3.68 (s, 3H, CO₂Me), 3.66-3.61 (m, 1H), 2.50 (dd, J=12.5, 2.4 Hz, 1H, C₂Hₐ), 2.45 (app. t, J=12.0 Hz, 1H, C₂H_{b}), 2.35 (s, 1H, C9-OH), 2.32 (t, J=7.2 Hz, 2H, C₄₂H₂), 2.14-2.05 (m, 5H), 2.05 (s, 3H, C7-OAc), 2.03-1.90 (m, 4H), 1.86 (ddd, J=14.0, 11.6, 3.0 Hz, 1H, C₂₄H_{b}), 1.77 (ddd, J=12.2, 4.6, 2.7 Hz, 1H, C₆H_{eq}), 1.67 (d, J=15.1 Hz, 1H, C₁₀H_{b}), 1.64-1.55 (m, 3H, C₄H_{b}, C₄₃H₂), 1.47 (app. q, J=12.0 Hz, 1H, C₆H_{b}), 1.41-1.28 (m, 4H, C₄₄H₂, C₄₅H₂), 1.22 (d, J=6.5 Hz, 3H, C₂₇H₃), 1.20 (s, 3H), 1.01 (s, 3H), 1.00 (s, 3H), 0.95 (s, 3H), 0.90 (t, J=7.2 Hz, 3H, C₄₆H₃); ¹³C-NMR (125 MHz, CDCl₃) δ 172.3, 170.9, 167.0, 152.4, 151.1, 143.5 (C13), 138.8, 130.0, 120.4, 109.2 (C30), 102.0, 98.9, 91.3 (C41), 80.2, 75.4, 73.8, 73.1, 72.9, 72.1, 70.3, 68.7, 65.9, 64.8, 51.3, 45.0, 42.8, 42.6, 42.1, 41.3, 41.1, 40.1, 35.8, 33.5, 31.3, 31.1 (C44), 29.9, 27.3 (C43), 24.6, 22.2 (C45), 21.3, 21.2, 19.9, 18.9 (C42), 17.0, 14.1 (C46); HRMS calculated for C₄₅H₆₆NaO₁₅ [M+Na]⁺: 869.4294; found 869.4290 (TOF ESI+).

### Synthetic Procedure for SUW203

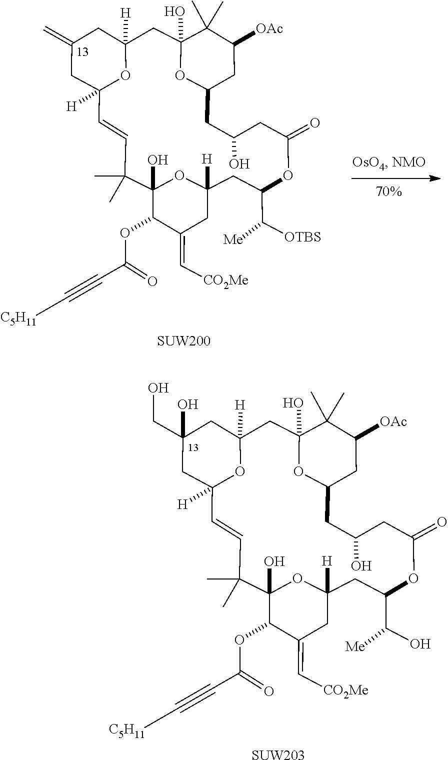

To a 1-dram vial was added SUW200 (6.6 mg, 0.0078 mmol, 1 equiv) in acetone (0.27 mL). NMO (1.4 mg, 0.0117 mmol, 1.5 equiv) and OsO₄ (30 μL of a 0.01 M solution in water, 0.00031 mmol, 0.04 equiv) were added sequentially and the resulting mixture was stirred for 24 h. The reaction mixture was diluted with EtOAc and sat. Na₂S₂O₃ and extracted with EtOAc (2×). The combined organic layers were dried over Na₂SO₄ and concentrated to a white paste. Purification by flash chromatography (pipette column, 5-10% MeOH/DCM) gave diol SUW203 as a white powder (4 mg, 60% yield). TLC: R_{f}=0.1 (80% EtOAc/hexane, UV active, purple spot in p-anisaldehyde) [α]23.9D=−9.8° (c=0.14, CH₂Cl₂); IR (thin film): 3447, 2928, 2858, 2234, 1735, 1718, 1654, 1412, 1366, 1245, 1158, 1106, 1080, 1063 cm⁻¹; ¹H NMR (600 MHz, CDCl₃) δ 5.96 (s, 1H), 5.71 (d, J=15.9 Hz, 1H), 5.26-5.18 (m, 2H), 5.12 (m, 3H), 4.22 (t, J=11.9 Hz, 1H), 4.14 (m, 1H), 4.06 (m, 1H), 3.99 (m, 2H), 3.86-3.76 (m, 2H), 3.75 (m, 1H), 3.68-3.66 (m, 4H), 2.49 (t, J=14.7 Hz, 2H), 2.30 (t, J=7.2 Hz, 2H), 2.05-1.94 (m, 6H), 1.92-1.51 (m, 22H), 1.48-1.34 (m, 6H), 1.33-1.26 (m, 6H), 1.23 (s, 3H), 1.21 (d, J=6.4 Hz, 3H), 1.16 (s, 3H), 0.98 (s, 3H), 0.97 (s, 3H), 0.91 (s, 3H), 0.87 (m 6H); ¹³C NMR (126 MHz, CDCl₃) δ 167.13, 151.20, 144.87, 142.48, 128.74, 128.66, 126.07, 120.58, 110.77, 102.10, 99.06, 95.85, 75.47, 73.27, 70.47, 68.81, 68.61, 68.18, 66.26, 66.01, 64.91, 51.44, 46.96, 45.11, 42.31, 41.85, 41.33, 40.14, 38.98, 32.46, 31.25, 30.61, 30.26, 27.43, 26.95, 24.74, 22.98, 22.36, 21.46, 21.29, 19.05, 17.15, 14.41, 14.17; HRMS calculated for C₄₅H₆₈O₁₇ [M+Na]⁺: 903.4349; found 903.4334 (TOF ESI+).

### Synthetic Procedure for SUW226

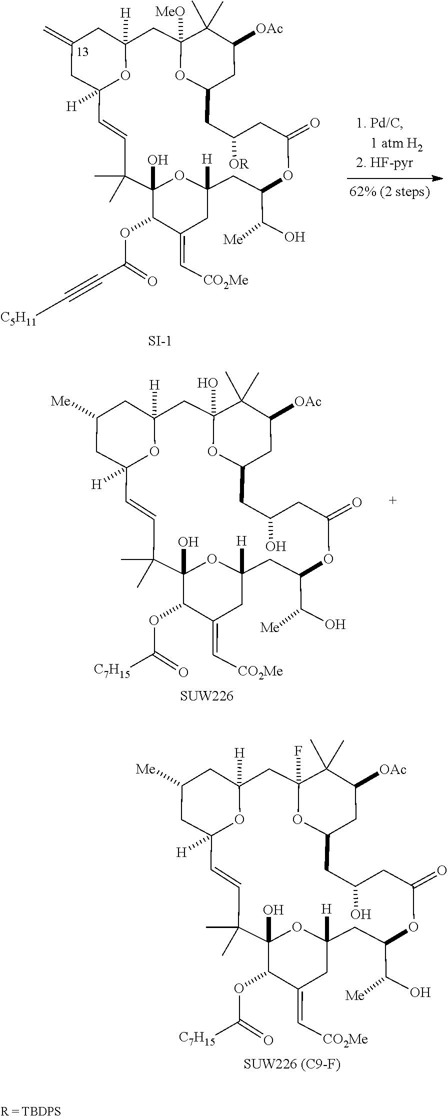

Hydrogenation: To an 8-dram vial equipped with magnetic stir bar was added Prins product SI-1 (Science, 2017, 358, 218-223) (50 mg), EtOAc (500 uL), and palladium on carbon (50 mg). The vial was placed under a hydrogen atmosphere (balloon). After 2 h, TLC analysis indicated complete conversion of starting material. The reaction mixture was filtered over a pipette containing celite and directly concentrated. Purification was accomplished by silica gel flash column chromatography (25% EtOAc/pentane) affording 40 mg of material.

Global deprotection: To a cooled (0° C.) solution of this material (40 mg) in 1:1 THF/pyridine (800 uL) was added 70% HF-pyridine (400 uL), affording a 1:2:2 HF-pyr/THF/pyridine solution. The reaction mixture was allowed to warm to room temperature. After 36 h, H₂O (400 uL) was added, and the reaction mixture was heated to 40° C. After 3 h at 40° C., the reaction mixture was cooled to 0° C., diluted with EtOAc (10 mL), and slowly quenched by adding saturated aqueous NaHCO₃ dropwise until bubbling ceased. The layers were separated, and the aqueous layer was extracted with 80% EtOAc/Hex (5×10 mL). The combined organic layers were dried over NaSO₄, filtered, and concentrated. Purification was accomplished by silica gel flash column chromatography (33-67% EtOAc/Hex) affording a mixture of SUW226 and its C9-fluoride (24 mg, 62% combined yield over 2 steps). 15 mg of this diastereomeric mixture was then subjected to RP-HPLC (70-100% MeCN/H₂O) affording pure samples of SUW226 and its C9-fluoride. The stereochemistry at C13 was assigned from 2D-ROESY data (cross-peaks observed between C₁₃CH₃ and C₁₁H, and between C₁₃CH₃ and C₁₅H). TLC R_{f}=0.31 (50% EtOAc/Hex, UV active, dark purple spot in p-anisaldehyde); [α]²³_{D}=18.2° (c=0.18, CH₂Cl₂); IR (thin film): 3450 (bs), 2928, 1738, 1720, 1437, 1366, 1237, 1157, 1099, 1060, 1002 cm⁻¹; ¹H-NMR (600 MHz, CDCl₃) δ 6.46 (d, J=2.0 Hz, 1H, C₃₄H), 6.24 (d, J=15.8 Hz, 1H, C₁₇H), 5.84 (s, 1H, C₁₉—OH), 5.74 (s, 1H, C₂₀H), 5.55 (dd, J=15.8, 8.2 Hz, 1H, C₁₆H), 5.40-5.36 (m, 1H, C₂₅H), 5.35 (dd, J=11.9, 4.9 Hz, 1H, C₇H), 4.72-4.65 (m, 1H, C₁₅H), 4.62 (d, J=12.3 Hz, 1H, C₃—OH), 4.53-4.45 (m, 1H, C₂₃H), 4.34 (dd, J=13.6, 2.2 Hz, 1H, C₂₂H_{eq}), 4.12 (app. t, J=11.6 Hz, 1H, C₃H), 4.08-4.01 (m, 1H, C₁₁H), 3.90-3.82 (m, 1H, C₅H), 3.68 (app. p, J=6.3 Hz, 1H, C₂₆H), 3.21 (s, 3H), 2.48-2.37 (m, 2H, C₂Ha, C₂₂Hₐₓ), 2.20-2.10 (m, 3H, C₂H_{b}, C₄₀H₂), 2.07 (dd, J=15.0, 7.1 Hz, 1H, C₁₀Hₐ), 1.94-1.87 (m, 1H, C₁₃H), 1.81-1.70 (m, 2H, C₂₄H₂), 1.68 (s, 3H), 1.55 (s, 3H), 1.28 (s, 3H), 1.24 (d, J=7.3 Hz, 3H, C₁₃CH₃), 1.00 (d, J=6.4 Hz, 3H, C₂₇H₃), 0.93 (s, 3H), 0.90 (s, 3H), 0.86 (t, J=7.2 Hz, 3H, C₄₆H₃); ¹³C-NMR (125 MHz, C₆D₆) δ 172.9, 171.8, 170.0, 166.8, 152.7, 138.6, 131.2, 120.5, 102.2, 99.8, 75.0, 74.4, 74.0, 73.0, 70.4, 68.8, 65.7, 65.5, 65.2, 50.6, 45.3, 42.8, 42.6, 41.2, 39.9, 39.8, 38.2, 36.2, 34.8, 33.7, 32.1, 32.0, 29.33, 29.27, 26.4, 25.4, 25.1, 23.0, 21.2, 20.7, 20.0, 19.8, 18.0, 17.0, 14.3; HRMS calculated for C₄₅H₇₂NaO₁₅ [M+Na]⁺: 875.4764; found 875.4737 (TOF ESI+).

### Procedure for Obtaining SUW201

SUW201 is the C13 (E)-enoate isomer of bryostatin 1. Because the HWE olefination produces a ˜10:1 mixture of C13 isomers, SUW201 can be separated from bryostatin 1 via RP-HPLC. See Science, 2017, 358, 218-223 for synthesis and purification conditions. [α]^{23.2}_{D}=−35.5° (c=0.26, CH₂Cl₂); IR (thin film): 3464, 3336, 2951, 2928, 1716, 1657, 1643, 1615, 1435, 1408, 1366, 1284, 1242, 1156, 1098, 1078, 1057, 1002, 859 cm⁻¹; ¹H-NMR (500 MHz, CDCl₃) δ 7.30-7.23 (dd obscured by chloroform peak, 1H, C₄₁H), 6.21-6.11 (m, 2H, C₄₂H, C₄₃H), 6.00 (d, J=1.9 Hz, 1H, C₃₄H), 5.81 (d, J=15.8 Hz, 1H), 5.80 (d, J=15.3 Hz, 1H), 5.71 (s, 1H, C₃₀H), 5.30 (dd, J=15.9, 8.4 Hz, 1H, C₁₆H), 5.24 (s, 1H, C₁₉—OH), 5.24-5.18 (m, 1H, C₂₅H), 5.20 (s, 1H, C₂₀NH), 5.15 (dd, J=11.8, 4.7 Hz, 1H, C₇H), 4.27 (d, J=12.1 Hz, 1H, C₃—OH), 4.28-4.20 (m, 1H, C₅H), 4.21-4.09 (m, 2H, C₃H, C₁₅H), 4.02 (app. t, J=11.4 Hz, 1H, C₂₃H), 3.86-3.78 (m, 1H, C₁₁H), 3.79-3.73 (m, 1H, C₂₆H), 3.74-3.67 (m, 2H, C₂₂H_{eq}, C₁₄H_{eq}), 3.69 (s, 3H, CO₂Me), 3.66 (s, 3H, CO₂Me), 2.67 (bs, 1H, C₉—OH), 2.53-2.38 (m, 2H, C₂H₂), 2.19-1.90 (m, 10H), 2.05 (s, 3H, C7-OAc), 1.86-1.79 (m, 1H, C₂₄H_{b}), 1.79-1.72 (m, 2H, C₆H_{eq}, C₁₀H_{b}), 1.63-1.56 (m, 1H, C₄H_{b}), 1.54-1.41 (m, 3H, C₆Hₐₓ, C₄₅H₂), 1.23 (d, J=6.5 Hz, 3H, C₂₇H₃), 1.14 (s, 3H), 1.004 (s, 3H), 1.001 (s, 3H), 0.95 (s, 3H), 0.92 (t, J=7.4 Hz, 3H, C₄₆H₃); ¹³C-NMR (125 MHz, CDCl₃) δ 172.4, 171.0, 167.2, 167.1, 165.7, 156.9, 152.0, 146.5, 145.7, 139.7, 129.2, 128.5, 119.8, 118.7, 114.5, 101.9, 99.1, 79.9, 74.2, 73.7, 73.0, 71.5, 70.3, 68.7, 65.9, 64.8, 51.2 (2C), 45.0, 43.2, 42.5, 42.2, 41.2, 40.0, 37.6, 35.9, 35.2, 33.5, 31.4, 24.7, 22.0, 21.3, 21.2, 19.9, 19.8, 17.0, 13.8; HRMS calculated for C₄₇H₆₈NaO₁₇ [M+Na]⁺: 927.4349; found 927.4338 (TOF ESI+).

### Synthesis of Modified C13 Enoates: HWE Olefination of C13 Ketone

Preparation of HWE Reagents from Diethylphosphonoacetic Acid:

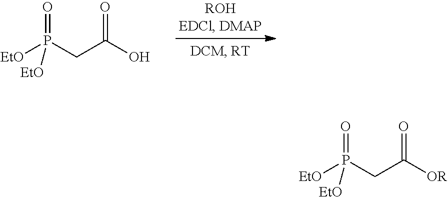

Chemicals:

Diethylphosphonoacetic acid (Aldrich, used without purification)

DMAP (4-dimethylaminopyridine) (Aldrich): recrystallized from hexanes

EDCI (1-(3-dimethylaminopropyl)-3-ethylcarbodimmide hydrochloride) (Chem-Impex): used without purification

To a flame-dried 8-dram vial equipped with magnetic stir bar was added diethylphosphonoacetic acid (1.0 equiv) in DCM (˜0.2 M). The corresponding alcohol (1.1 equiv) was added, followed by DMAP (0.5 equiv) in a single portion. EDCI (2.0 equiv) was then added in a single portion and the reaction was stirred at RT for 30 minutes, at which point TLC indicated complete conversion of starting material. The reaction mixture was directly diluted with water and extracted with 3×EtOAc. The combined organic layers were washed with brine and dried over Na₂SO₄, filtered, and concentrated. The crude residue was purified by silica gel flash chromatography (80-90% EtOAc in hexanes) to afford the desired phosphonate, which was then used in the subsequent HWE olefination. Compound purity was established by TLC (one spot) analysis. Characterization data matched literature values reported by Lloyd et al. (allyl phosponate, Organic and Biomolecular Chemistry 2016, 14, 8971-8988.) and O'Leary et al. (benzyl phosphonate, JACS 2001, 123, 11519-33).

Note that exact amounts of phosphonates prepared varied, but procedure was generally carried out on a ˜200 mg scale.

HWE Olefination of C13 Ketone:

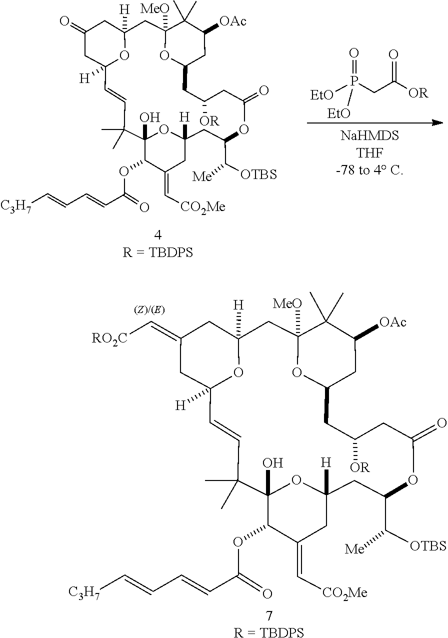

Chemicals:

NaHMDS (sodium hexamethyldisilazide, 1M in THF, Aldrich): used without purification

Triethylphosphonate (Aldrich): used without purification

Alternative phosphoates prepared via procedure presented above.

To a flame-dried 8-dram vial equipped with a magnetic stir bar was added the corresponding phosphonate (azeotroped in benzene×2) in THF. The solution was cooled to 0° C. and NaHMDS was added dropwise. The reaction mixture was allowed to stir at 0° C. for 30 minutes, at which point it became bright yellow. Separately, ketone 4 (azeotroped in benzene×2) was added to a flame-dried 8-dram vial equipped with a magnetic stir bar in THF. The solution was cooled to −78° C. and an aliquot of the deprotonated phosphonate was added dropwise as a solution in THF. The reaction was allowed to stir at −78° C. for 5 minutes, at which point it was transferred to a cold room and stirred at 4° C. for ˜2 hours, at which point TLC indicated complete consumption of starting material. The reaction mixture then was pipetted in to sat. aq. NH₄Cl and extracted with 3×Et₂O. The combined organic layers were dried over Na₂SO₄, filtered, and concentrated. The crude residue was purified by silica gel flash chromatography (15-25% EtOAc in hexanes; TLC R_{f}=0.52 in 60% EtOAc/hexanes, purple spot p-anisaldehyde) to afford the desired C13 enoates as a mixture of (Z) and (E) isomers which are inseparable on silica gel. The mixture of geometric isomers was moved directly in to the subsequent global deprotection.

Synthesis of Modified C13 Enoates: Global Deprotection

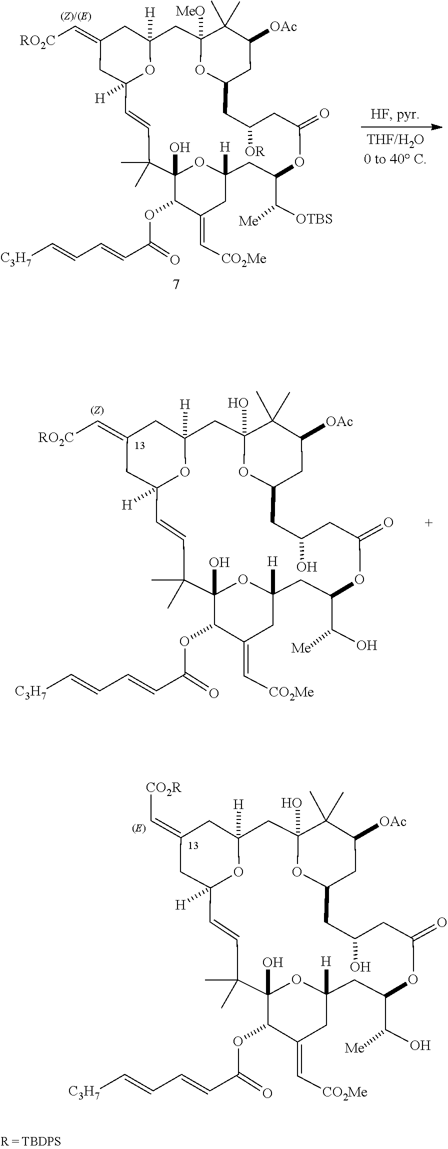

Chemicals

70% HF-pyridine (Sigma-Aldrich): used without purification

Pyridine (Sigma-Aldrich): distilled from CaH₂ before use

To a 15 mL polypropylene falcon tube equipped with a magnetic stir bar was added enoate 7 in THF. The solution was cooled to 0° C. and pyridine was added dropwise. HF-pyridine was then added dropwise, affording a 0.0075 M solution of enoate 7 in a 1:2:2 mixture of HF-pyridine/THF/pyridine, and the resulting reaction mixture was directly transferred to a 40° C. oil bath. The reaction was allowed to stir for 20 hrs., at which point H₂O (equal volume to HF-pyridine) was added dropwise. The reaction mixture was allowed to stir for an additional 2 hrs at 40° C. The reaction mixture was then directly syringed in to the aqueous layer of a pre-chilled mixture of EtOAc/sat. aq. NaHCO₃. The aqueous layer was extracted with 3×EtOAc and the combined organic layers were washed with 1 M HCl followed by brine. The combined organic layers were then dried over Na₂SO₄, filtered, and concentrated. The product was purified by silica gel flash chromatography (10-35-45-50-60% EtOAc in hexanes), affording a mixture of C13 geometric isomers, which were subsequently separated by HPLC.

Preparative HPLC

˜1:1 Z:E mixture of C13 enoate isomers was further purified by preparative HPLC using a Shimadzu LC-20AP Prominence preparative liquid chromatograph system with detectors set to 254 and 280 nm. Separations were performed using a Restek Ultra C18 column (5 m particle size, 250 mm×10 mm). The mobile phase was a gradient elution from 75% MeCN/H2O to 100% MeCN/H2O over 30 min, followed by 100% MeCN for 10 min (flow rate of 5 mL/min). The sample was dissolved in 1:1 MeCN/MeOH for loading. Two fractions were collected, affording diastereomerically pure samples of the (Z)- and (E)-enoate isomers as a fluffy white powder (fraction 1: (Z) enoate, fraction 2: (E) enoate). Sterochemistry of the C13 enoate was confirmed by ROESY NMR on SUW218 (more details below). Additionally, HPLC retention times and relative C30 1H chemical shifts matched pattern observed for C13 (Z) and (E)-methyl enoates of bryostatin 1/SUW201 (Science 2017, 358, 218-223).

Characterization data for SUW217-SUW220, SUW229, and SUW230 provided below.

Characterization data for SUW217: TLC R_{f}=0.31 (60% EtOAc/pentane, purple spot in p-anisaldehyde); [α]^{25.0}_{D}=24.0° (c=0.08, CH₂Cl₂); IR (thin film) 3463, 3341, 2926, 2854, 1653, 1436, 1365, 1247, 1003, 860 cm⁻¹; ¹H NMR (600 MHz, CDCl₃) δ 7.29-7.24 (m, 1H) 6.19-6.15 (m, 2H), 6.01 (d, J=2.1 Hz, 1H), 5.81 (d, J=3.8 Hz, 1H), 5.79 (d, J=4.3 Hz, 1H), 5.67 (s, 1H), 5.31 (dd, J=15.7, 8.4 Hz, 1H), 5.24-5.18 (m, 3H), 5.15 (dd, J=11.9, 4.7 Hz, 1H), 4.22 (t, J=11.6 Hz, 1H), 4.16 (q, J=7.1 Hz, 3H), 4.08 (app t, J=9.7 Hz, 1H), 4.02 (app t, J=11.3 Hz, 1H), 3.84-3.74 (m, 2H), 3.71-3.64 (m, 1H), 3.67 (s, 3H), 2.49 (d, J=10.8 Hz, 1H), 2.43 (t, J=11.8 Hz, 1H), 2.34 (br s, 1H), 2.21 (t, J=12.4 Hz, 1H), 2.18-2.12 (m, 2H), 2.12-2.06 (m, 2H), 2.05 (s, 3H), 2.05-1.92 (m, 1H), 1.91 (t, J=12.8 Hz, 1H), 1.83 (t, J=12.3 Hz, 1H), 1.79-1.74 (m, 1H), 1.67 (d, J=15.1 Hz, 1H), 1.60 (app d, J=15.1 Hz, 1H), 1.51-1.42 (m, 3H), 1.28 (t, J=7.1 Hz, 4H), 1.27-1.21 (m, 12H), 1.15 (s, 3H), 1.01 (s, 6H), 0.95 (s, 3H), 0.92 (t, J=7.4 Hz, 3H); ¹³C NMR (126 MHz, CDCl₃) δ 172.31, 170.97, 167.16, 166.43, 165.74, 156.15, 152.11, 146.52, 145.67, 139.37, 129.56, 128.51, 119.72, 118.74, 115.08, 101.94, 99.14, 79.28, 74.18, 73.82, 72.86, 71.60, 70.31, 68.60, 65.97, 64.83, 59.93, 51.23, 45.05, 44.25, 42.53, 42.12, 41.09, 39.98, 36.43, 36.00, 35.21, 33.46, 32.08, 31.43, 24.73, 22.02, 21.20, 19.97, 16.96, 14.44, 13.85, 1.18; HRMS calculated for C₄₈H₇₀NaO₁₇ [M+Na]⁺: 941.4505; found 941.4478 (TOF ESI+).

Characterization data for SUW218: TLC R_{f}=0.31 (60% EtOAc/pentane, purple spot in p-anisaldehyde); [α]^{24.7}_{D}=6.66 (c=0.13, CH₂Cl₂); IR (thin film) 3463, 3343, 2927, 2854, 1734, 1716, 1644, 1435, 1241, 1146, 1003 cm⁻¹; ¹H NMR (600 MHz, CDCl₃) δ 7.30-7.23 (dd obscured by chloroform peak, 1H), 6.19-6.13 (m, 2H), 6.01 (d, J=1.9 Hz, 1H), 5.82 (d, J=9.8 Hz, 1H), 5.79 (d, J=9.2 Hz, 1H), 5.70 (s, 1H), 5.30 (dd, J=15.7, 8.5 Hz, 1H), 5.23 (s, 1H), 5.23-5.20 (m, 1H), 5.20 (s, 1H), 5.15 (dd, J=11.8, 4.8 Hz, 1H), 4.25 (m, 2H), 4.19-4.10 (m, 4H), 4.03 (t, J=11.2 Hz, 1H), 3.82 (p, J=6.3 Hz, 1H), 3.76-3.67 (m, 3H), 3.67 (s, 3H), 2.49 (dd, J=12.4, 2.8 Hz, 1H), 2.45 (q, J=12.5, 11.4 Hz, 1H), 2.19-2.06 (m, 5H), 2.04 (s, 3H), 2.03-1.91 (m, 3H), 1.87-1.79 (m, 1H), 1.79-1.75 (m, 1H), 1.76 (d, J=15.2 Hz, 1H), 1.51-1.41 (m, 3H), 1.28 (t, J=7.1 Hz, 3H), 1.26 (s, 3H), 1.23 (d, J=6.5 Hz, 3H), 1.14 (s, 3H), 1.01 (s, 3H), 1.00 (s, 3H), 0.95 (s, 3H), 0.92 (t, J=7.4 Hz, 3H), 0.88 (t, J=7.0 Hz, 1H); ¹³C NMR (126 MHz, CDCl₃) δ 172.39, 170.93, 167.14, 166.79, 165.70, 156.47, 152.00, 146.52, 145.70, 139.70, 129.22, 128.50, 119.79, 118.73, 114.96, 101.88, 99.13, 79.91, 74.15, 73.70, 72.95, 71.50, 70.24, 68.72, 65.91, 64.84, 59.97, 51.24, 45.03, 43.14, 42.56, 42.20, 41.16, 40.04, 37.58, 35.90, 35.21, 33.51, 31.42, 24.73, 22.02, 21.33, 21.20, 19.91, 19.83, 16.94, 14.43, 13.85; HRMS calculated for C₄₈H₇₀NaO₁₇ [M+Na]⁺: 941.4505; found 941.4485 (TOF ESI+).

Characterization data for SUW219: TLC: R_{f}=0.32 (50% EtOAc/pentane, UV active, purple spot by p-anisaldehyde stain); [α]^{23.2}=+5.9° (c=0.1, CH₂Cl₂); IR (thin film): 3467, 3314, 2927, 1717, 1654, 1647, 1458, 1437, 1364, 1287, 1246, 1161, 1079, 1058, 1027, 1004, and 860 cm⁻¹; ¹H-NMR (600 MHz, CDCl₃): δ 7.37-7.31 (m, 5H, phenyl), 7.29-7.25 (m, 1H, obscured by residual chloroform peak, C₄₁H), 6.18-6.16 (m, 2H, C₄₂H, and C₄₃H), 6.01 (d, 1H, J=1.8 Hz, C₃₄H), 5.81 (d, 1H, J=15.2 Hz, C₄₀H), 5.80 (d, 1H, J=15.8 Hz, C₁₇H), 5.74 (t, 1H, J=1.6 Hz, C₃₀H), 5.31 (dd, 1H, J=15.8, 8.4 Hz, C₁₆H), 5.23-5.11 (m, 6H), 4.25-4.15 (m, 3H, C3-OH, C₃H, C₅H), 4.09 (ddd, 1H, J=11.0, 8.5, 2.2 Hz, C₁₅H), 4.02 (tt, 1H, J=11.5, 2.2 Hz, C₂₂H), 3.83-3.74 (m, 2H), 3.71 (s, 1H), 3.68 (s, 1H), 3.67 (s, 3H, CO₂Me), 2.50-2.41 (m, 2H), 2.36 (s, 1H), 2.13 (t, 1H, J=12.4 Hz), 2.18-2.14 (m, 2H), 2.12 (dd, 1H, J=7.8, 15.2 Hz), 2.07 (bs, 1H), 2.05 (s, 3H, C₇OAc), 2.03-1.97 (m, 2H), 1.94-1.89 (m, 2H), 1.86-1.81 (m, 1H), 1.76 (ddd, 1H, J=2.7, 4.5, 12.5 Hz), 1.59 (dt, 1H, J=3.1, 14.9 Hz), 1.51-1.43 (m, 4H), 1.24 (d, 3H, J=6.5 Hz, C₂₇H), 1.15 (s, 3H), 1.01 (s, 6H), 0.95 (s, 3H), and 0.93 (t, 3H, J=7.4 Hz, C₄₆H) ppm; ¹³C-NMR (125 MHz, CDCl₃): δ 172.41, 171.00, 167.25, 166.23, 165.84, 156.96, 152.20, 146.63, 145.78, 139.54, 136.35, 129.60, 128.81, 128.62, 128.41, 128.40, 128.39, 128.17, 119.83, 118.84, 114.93, 102.03, 99.24, 79.36, 74.23, 73.94, 72.90, 71.65, 70.41, 68.71, 66.11, 65.98, 64.93, 51.33, 45.16, 44.35, 42.65, 42.24, 41.18, 40.06, 36.58, 36.09, 35.32, 33.55, 31.52, 24.83, 22.12, 21.42, 21.29, 20.09, 20.03, 17.06, and 13.96 ppm; HRMS: Calcd for C₅₃H₇₂O₁₇Na [M+Na⁺]: 1003.4662, found 1003.4649.

Characterization data for SUW220: TLC: R_{f}=0.38 (50% EtOAc/pentane, UV active, purple spot by p-anisaldehyde stain); [α]^{23.2}_{D}=−6.8° (c=0.1, CH₂Cl₂); IR (thin film): 3466, 3352, 2961, 2928, 1715, 1642, 1455, 1435, 1408, 1366, 1283, 1241, 1260, 1141, 1098, 1078, 1056, 1027, and 1004 cm⁻¹; ¹H-NMR (600 MHz, CDCl₃): δ 7.37-7.30 (m, 5H, Ph), 7.28-7.24 (m, 1H, obscured by the chloroform peak, C₄₁H), 6.17-6.16 (m, 2H, C₄₂H, C₄₃H), 6.01 (s, 1H, C₃₄H), 5.81 (d, 1H, J=15.7 Hz), 5.79 (d, 1H, J=15.3 Hz), 5.77 (s, 1H, C₃₀H), 5.30 (dd, 1H, J=8.1, 15.7 Hz, C₁₆H), 5.24-5.11 (m, 6H), 4.26 (d, 1H, J=12.0 Hz, C₃—OH), 4.24 (tt, 1H, J=3.0, 12.0 Hz, C₅H), 4.22 (m, 2H), 4.19-4.12 (m, 2H), 4.02 (t, 1H, J=11.2 Hz, C₂₃H), 3.82 (app. hextet, 1H, J=6.0, C₂₆H), 3.76-3.68 (m, 2H), 3.67 (s, 3H, CO₂Me), 2.49-2.44 (m, 3H), 2.17-2.06 (m, 6H), 2.05 (s, 3H, C₇—OAc), 2.03-1.93 (m, 2H), 1.91-1.73 (m, 4H), 1.62-1.59 (m, 2H), 1.31-1.25 (m, 2H), 1.20 (d, J=6.4 Hz, 3H, C₂₇H), 1.11 (s, 3H), 0.98 (bs, 6H), 0.92 (s, 3H), and 0.89 (t, 3H, J=7.4 Hz) ppm; ¹³C-NMR (125 MHz, CDCl₃): δ 172.51, 171.03, 167.24, 166.58, 165.80, 157.39, 152.09, 146.63, 145.81, 139.88, 136.35, 129.26, 128.82, 128.60, 128.42, 128.39, 119.89, 118.83, 114.73, 101.99, 99.23, 80.00, 77.47, 74.25, 73.80, 73.04, 71.58, 70.34, 68.82, 66.03, 65.98, 64.95, 51.35, 45.14, 43.31, 42.65, 42.29, 41.27, 40.13, 37.77, 35.99, 33.60, 32.19, 31.51, 24.82, 22.60, 22.12, 21.44, 21.31, 20.02, 19.93, 17.05, and 13.95 ppm; HRMS: Calcd for C₅₃H₇₂O₁₇Na⁺ [M+Na⁺]: 1003.4662, found 1003.4650.

Characterization data for SUW229: TLC R_{f}=0.38 (60% EtOAc/hexanes, purple spot in p-anisaldehyde); [α]^{24.7}_{D}=7.66 (c=0.07, CH₂Cl₂); IR (thin film) 3460, 3355, 3312, 2959, 2919, 2580, 1717, 1662, 1634, 1261, 1157, 1023 cm⁻¹; ¹H NMR (600 MHz, CDCl₃) δ 7.30-7.23 (dd obscured by chloroform peak, 1H), 6.19-6.15 (m, 2H), 6.01 (s, 1H), 5.95 (m, 1H), 5.81 (s, 1H), 5.79 (s, 1H), 5.72 (s, 1H, C30 H), 5.37-5.28 (m, 2H), 5.27-5.17 (m, 3H), 5.20 (s, 1H) 5.14 (d, J=12.1 Hz, 1H), 4.62 (appf s, 2H), 4.26-4.13 (m, 3H), 4.08 (app. t, J=10.0 Hz, 1H), 4.02 (app. t, J=12.8 Hz, 1H), 3.82 (m, 1H), 3.77 (m, 1H), 3.70 (m, 1H), 3.69-3.66 (m, 1H) 3.67 (s, 3H), 2.49 (app d, J=12.5 Hz), 2.43 (app t, J=11.5 Hz, 1H), 2.05 (s, 3H), z 1.15 (s, 3H), 1.01 (s, 6H), 0.96 (s, 3H), 0.92 (t, J=7.4 Hz, 3H); ¹³C NMR (126 MHz, CDCl₃) δ 172.39, 170.93, 167.11, 165.99, 165.73, 152.09, 146.52, 139.43, 132.49, 129.50, 128.51, 128.42, 119.73, 118.74, 118.22, 114.73, 101.94, 99.14, 79.29, 74.17, 73.87, 72.83, 71.58, 70.29, 68.59, 65.99, 64.82, 64.70, 51.31, 51.23, 45.06, 44.26, 42.52, 42.13, 41.08, 39.95, 36.47, 35.22, 32.08, 29.85, 24.72, 22.85, 22.02, 21.33, 21.19, 19.96, 16.96, 14.29, 13.85; HRMS calculated for C₄₉H₇₀O₁₇ [M+Na⁺]: 953.4505; found 953.4475 (TOF ESI+).

Characterization data for SUW230: TLC R_{f}=0.46 (60% EtOAc/hexanes, purple spot in p-anisaldehyde); [α]^{25.0}_{D}=−30.10 (c=0.07, CH₂Cl₂); IR (thin film) 3463, 3358, 2962, 2918, 2850, 1719, 1653, 1559, 1540, 1261, 1080, 1026 cm⁻¹; ¹H NMR (600 MHz, CDCl₃) δ 7.30-7.23 (dd obscured by chloroform peak, 1H), 6.18-6.14 (m, 2H), 6.01 (s, 1H), 5.98-5.88 (m, 1H), 5.81 (app t, J=14.0 Hz, 2H), 5.75 (s, 1H, C30 H), 5.33 (d, J=17.4 Hz, 1H), 5.31-5.18 (m, 3H), 5.22 (s, 1H), 5.20 (s, 1H), 5.15 (d, J=13.4 Hz, 1H), 4.60 (app s, 2H), 4.29-4.21 (m, 2H), 4.21-4.12 (m, 2H), 4.03 (t, J=10.7 Hz, 1H), 3.82 (m, 1H), 3.76-3.68 (m, 3H), 3.67 (s, 3H), 2.52-2.41 (m, 2H), 2.33 (s, 1H), 2.04 (s, 3H), 1.15 (s, 3H), 1.01 (s, 6H), 0.95 (s, 3H), 0.92 (t, J=7.8 Hz, 3H); ¹³C NMR (126 MHz, CDCl₃) δ 172.41, 170.91, 167.14, 166.36, 165.70, 157.20, 151.99, 146.52, 145.70, 139.76, 132.45, 129.17, 128.50, 119.79, 118.73, 118.26, 114.57, 101.88, 99.13, 74.14, 73.71, 72.93, 71.50, 70.24, 68.72, 65.93, 64.84, 64.73, 51.24, 45.03, 43.18, 42.55, 42.18, 41.17, 40.02, 37.62, 35.88, 35.21, 33.49, 31.41, 29.86, 24.72, 22.02, 21.33, 21.19, 19.91, 19.83, 16.94, 13.85; HRMS calculated for C₄₉H₇₀O₁₇ [M+Na⁺]: 953.4505; found 953.4479 (TOF ESI+).

Synthetic Procedure for SUW204

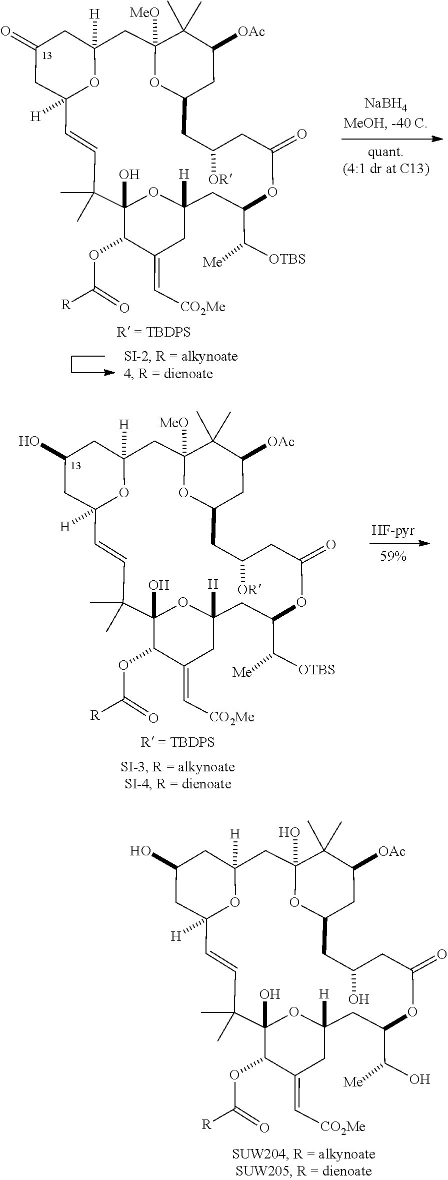

Reduction: To a cooled (−20° C.) solution of SI-2 (14 mg, 0.012 mmol, 1 equiv) in MeOH (1.15 mL, 0.01M) was added sodium borohydride (0.5 mg, 0.013 mmol, 1.15 equiv). After 2 h, TLC analysis indicated full conversion of starting material. The now yellow reaction mixture was quenched at −20° C. by adding saturated aqueous NH₄Cl (2 mL). The layers were separated, and the aqueous layer was extracted with 50% EtOAc/Hex (5×3 mL). The combined organic layers were dried over NaSO₄, filtered, and concentrated. Purification was accomplished by flash column chromatography using a 1.7×6 cm silica gel column, eluting with 15-25% EtOAc/Hex, and collecting 4 mL fractions. Frxns #20-26 afforded 11.6 mg (83% yield) of SI-3 and frxns #27-31 afforded 2 mg (14% yield) of its C13 diastereomer (structure not shown). The relative stereochemistry at C13 was assigned after C13-acetylation to afford SUW206 and SUW207, respectively. TLC of SI-3: R_{f}=0.44 (30% EtOAc/Hex, UV active); TLC of C13 diastereomer of SI-3: R_{f}=0.63 (30% EtOAc/Hex, UV active).

Global deprotection: To a cooled (0° C.) solution of SI-3 (11.6 mg, 0.01 mmol, 1 equiv) in 3:1 THF/H₂O (1 mL, 0.01M) was added 70% HF-pyridine (300 uL). The reaction mixture was allowed to warm to room temperature. After 48 h, TLC analysis indicated incomplete deprotection so additional 70% HF-pyridine (150 uL; total of 450 uL) was added. After an additional 24 h (total reaction time of 72 h), the reaction mixture was cooled to 0° C., diluted with 50% EtOAc/Hex (5 mL), and slowly quenched by adding saturated aqueous NaHCO₃ dropwise until bubbling ceased. The layers were separated, and the aqueous layer was extracted with 80% EtOAc/Hex (5×5 mL). The combined organic layers were dried over NaSO₄, filtered, and concentrated. Purification was accomplished by silica gel flash column chromatography (25% EtOAc/Hex to remove silanol, then 50-100% EtOAc/Hex), followed by RP-HPLC (70-100% MeCN/H₂O) affording SUW204 (4.8 mg, 59% yield). The same reduction/deprotection sequence was repeated with dienoate 4 to afford SI-4 and ultimately SUW205.

Characterization data for SUW204: TLC R_{f}=0.27 (100% EtOAc, not UV active, stains in CAM); [α]²⁴_{D}=6.8° (c=0.15, CH₂Cl₂); IR (thin film): 3454 (bs), 2927, 2855, 1715, 1366, 1286, 1160, 1058 cm⁻¹; ¹H-NMR (600 MHz, C₆D₆) δ 6.43 (s, 1H, C₃₄H), 6.19 (d, J=15.8 Hz, 1H, C₁₇H), 5.75 (s, 1H, C₂₀H), 5.50 (dd, J=15.8, 8.3 Hz, 1H, C₁₆H), 5.39-5.30 (m, 2H, C₂₅H, C₇H), 4.42 (app. t, J=11.8 Hz, 1H, C₂₃H), 4.30 (app. t, J=10.4 Hz, 1H, C₁₅H), 4.26 (app. d, J=13.9 Hz, 1H, C₂₂H_{eq}), 4.11-4.04 (m, 1H, C₃H), 3.86 (app. t, J=12.1 Hz, 1H, C₅H), 3.75-3.68 (m, 1H, C₁₃H), 3.68-3.61 (m, 2H, C₁₁H, C₂₆H), 3.21 (s, 3H), 2.47-2.37 (m, 2H, C₂Hₐ, C₂₂Hₐ), 2.18 (d, J=12.2 Hz, 1H, C₂H_{b}), 2.07 (dd, J=15.0, 6.9 Hz, 1H, C₁₀Hₐ), 1.69 (s, 3H), 1.66 (s, 3H), 1.03 (d, J=6.3 Hz, 3H, C₂₇H₃), 0.90 (s, 3H), 0.86 (s, 3H), 0.71 (t, J=6.4 Hz, 3H, C₄₆H₃); ¹³C-NMR (125 MHz, C₆D₆) δ 173.1, 170.2, 166.6, 152.5, 151.7, 139.0, 130.2, 121.1, 102.1, 99.7, 91.0, 77.8, 76.2, 74.5, 74.0, 73.0, 70.4, 69.6, 68.7, 67.5, 65.7, 65.4, 50.7, 45.4, 43.6, 42.5, 42.4, 42.4, 41.2, 39.8, 36.0, 33.6, 32.0, 31.8, 31.1, 27.2, 25.4, 22.3, 21.2, 20.7, 19.9, 18.6, 16.9, 14.0; HRMS calculated for C₄₄H₆₆NaO₁₆ [M+Na]⁺: 873.4243; found 873.4215 (TOF ESI+).

Synthetic Procedure for SUW206 and SUW207

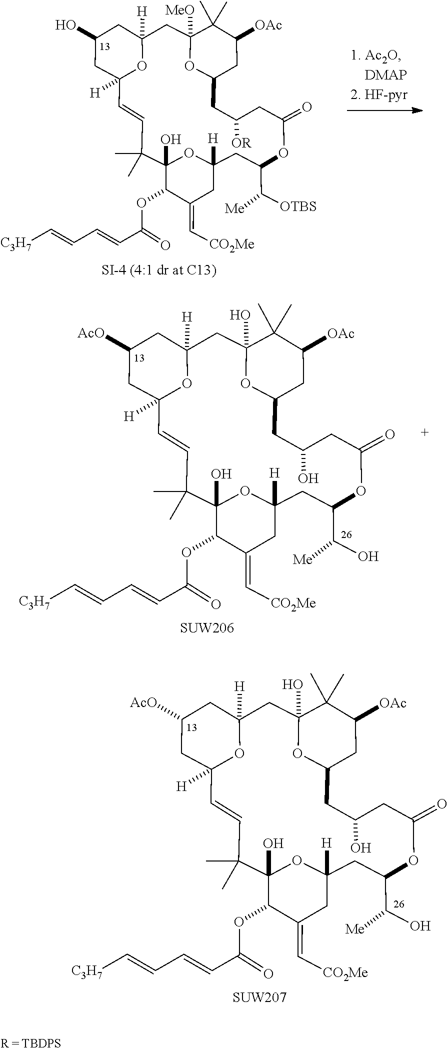

Acylation: To a solution of alcohol SI-4 (19 mg, ˜4:1 dr at C13, 0.016 mmol, 1 equiv) in CH₂Cl₂ (156 uL, 0.1M) was sequentially added DMAP (1 crystal) and acetic anhydride (2 drops). After 3 h, TLC analysis indicated complete conversion of starting material. The reaction mixture was directly flashed via silica gel flash column chromatography (20-30% EtOAc/Hex) affording the C13-OAc (quant., ˜4:1 dr at C13).

Global deprotection: To a cooled (0° C.) solution of this ester (19 mg, ˜4:1 dr at C13, 0.016 mmol, 1 equiv) in 1:1 THF/pyridine was added 70% HF-pyridine (˜0.0075M solution of 1:2:2 HF-pyr/THF/pyridine). The reaction mixture was allowed to warm to room temperature. After 20 h, H₂O (equal volume as HF-pyridine) was added, and the reaction mixture was heated to 40° C. After 3 h, the reaction mixture was cooled to 0° C., diluted with EtOAc, and slowly quenched by adding saturated aqueous NaHCO₃ dropwise until bubbling ceased. The layers were separated, and the aqueous layer was extracted with 50% EtOAc/Hex. The combined organic layers were dried over NaSO₄, filtered, and concentrated. Purification was accomplished by silica gel flash column chromatography (20% EtOAc/Hex to remove silanol, then 50-80% EtOAc/Hex), followed by RP-HPLC (70-100% MeCN/H₂O) affording SUW206 (6.6 mg, 47% yield) and SUW207 (1.4 mg, 10% yield). The relative stereochemistry at C13 was based on numerous literature examples demonstrating that axial protons are upfield of equatorial ones, e.g., for SUW206, axial proton at C13 observed at 4.97 ppm, whereas for SUW207, equatorial proton was observed at 5.13 ppm.

Characterization data for SUW206 (major C13 diastereomer): TLC R_{f}=0.58 (75% EtOAc/Hex, UV active, dark blue spot in p-anisaldehyde); [α]²³=3.4° (c=0.13, CH₂Cl₂); IR (thin film): 3452 (bs), 2927, 1735, 1718, 1246, 1157, 1075 cm⁻¹; ¹H-NMR (600 MHz, CDCl₃) δ 6.21-6.11 (m, 2H, C₄₂H, C₄₃H), 6.00 (s, 1H, C₃₄H), 5.79 (app. d, J=15.6 Hz, 2H, C₄₀H, C₁₇H), 5.26 (dd, J=15.9, 8.2 Hz, 1H, C₁₆H), 5.24-5.16 (m, 1H, C₂₅H), 5.19 (app. s, 2H, C₁₉—OH, C₂₀H), 5.17-5.12 (m, 1H, C₇H), 5.02-4.94 (m, 1H, C₁₃H), 4.32-4.20 (m, 2H, C₃—OH, C₅H), 4.19-4.10 (m, 2H, C₃H, C₁₅H), 4.03 (app. t, J=11.2 Hz, 1H, C₂₃H), 3.87-3.78 (m, 2H, C₂₆H, C₁₁H), 3.73-3.64 (m, 1H, C₂₂H_{eq}), 3.67 (s, 3H), 2.04 (s, 3H), 2.02 (s, 3H), 1.25 (d, J=6.0 Hz, 3H, C₂₇H₃), 1.12 (s, 3H), 0.99 (s, 6H), 0.95-0.89 (m, 6H); ¹³C-NMR (125 MHz, CDCl₃, 1 signal is obscured by solvent) δ 172.5, 170.9, 170.6, 167.1, 165.7, 152.1, 146.5, 145.7, 139.3, 129.1, 128.5, 119.8, 118.8, 101.9, 99.1, 74.1, 73.8, 72.9, 70.2, 69.9, 69.3, 68.6, 65.9, 64.8, 51.2, 45.0, 42.5, 42.0, 41.1, 40.0, 39.3, 37.9, 35.9, 35.2, 33.5, 31.4, 24.7, 22.0, 21.4, 21.3, 21.1, 19.9, 19.8, 17.0, 13.8; HRMS calculated for C₄₆H₆₈NaO₁₇ [M+Na]⁺: 915.4349; found 915.4322 (TOF ESI+).

Characterization data for SUW207 (minor C13 diastereomer): TLC R_{f}=0.43 (75% EtOAc/Hex, UV active, dark blue spot in p-anisaldehyde); [α]²⁴_{D}=3.1° (c=0.07, CH₂Cl₂); IR (thin film): 3463 (bs), 2931, 1743, 1719, 1244, 1158, 1099, 1057, 1029, 1003 cm⁻¹; ¹H-NMR (600 MHz, CDCl₃) δ 6.20-6.12 (m, 2H, C₄₂H, C₄₃H), 6.01 (s, 1H, C₃₄H), 5.80 (d, J=15.1 Hz, 1H), 5.75 (d, J=15.8 Hz, 1H), 5.32-5.07 (m, 6H), 4.38 (app. t, J=10.3 Hz, 1H), 4.34-4.29 (m, 1H), 4.26 (app. t, J=11.4 Hz, 1H), 4.22-4.15 (m, 1H), 4.09-4.00 (m, 2H), 3.88-3.80 (m, 1H), 3.72-3.68 (m, 1H), 3.67 (s, 3H), 2.17 (s, 3H), 2.05 (s, 3H), 1.24 (d, J=5.7 Hz, 3H, C27H₃), 1.13 (s, 3H), 1.00 (s, 6H), 0.96-0.89 (m, 6H); ¹³C-NMR (125 MHz, C₆D₆, 2 signals are obscured by solvent) δ 173.1, 169.9, 169.8, 166.8, 165.6, 152.7, 146.6, 145.1, 139.5, 120.6, 119.3, 102.0, 99.9, 74.9, 74.5, 74.3, 72.8, 70.5, 68.8, 67.8, 66.0, 65.8, 65.3, 50.6, 45.5, 42.5, 42.3, 41.2, 39.7, 37.5, 36.4, 36.1, 35.1, 33.6, 32.1, 25.3, 22.0, 21.2, 21.1, 20.7, 20.1, 19.8, 17.0, 13.7; HRMS calculated for C₄₆H₆₈NaO₁₇ [M+Na]⁺: 915.4349; found 915.4320 (TOF ESI+).

Synthetic Procedure for SUW208

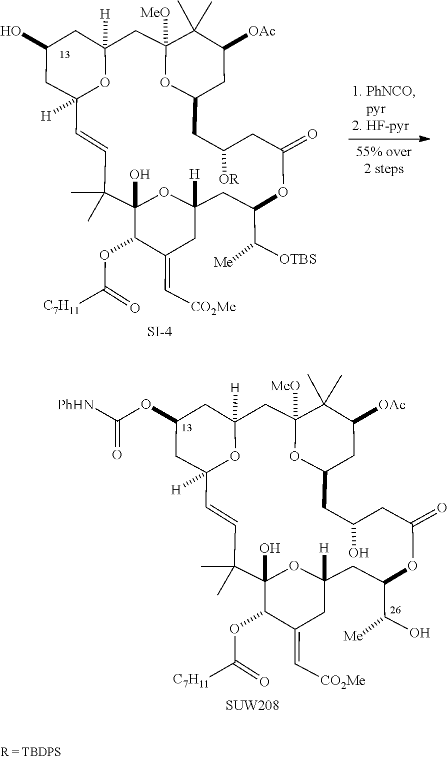

Ref: A. B. Smith et al., Design, Synthesis, and Evaluation of Carbamate-Substituted Analogues of (+)-Discodermolide. Org. Lett. 2005, 7, 311-314.

Acylation: To a solution of SI-4 (15 mg, 0.012 mmol, 1 equiv) in 2:1 CH₂Cl₂/pyridine (1.23 mL, 0.01M) was added phenyl isocyanate (40 uL, 0.369 mmol, 30 equiv). After 48 h, the reaction mixture was quenched by adding saturated aqueous NH₄Cl (2 mL). The layers were separated, and the aqueous layer was extracted with 80% EtOAc/Hex (3×5 mL). The combined organic layers were dried over NaSO₄, filtered, and concentrated. Purification was accomplished by silica gel flash column chromatography (20% EtOAc/Hex) affording the C13 carbamate (assume quantitative yield). LRMS calculated for C₇₄H₁₀₅NNaO₁₇Si₂ [M+Na]⁺: 1358.7; found 1359.0 (TOF ESI+).

Global deprotection: To a cooled (0° C.) solution of this carbamate (assume 0.012 mmol, 1 equiv) in 1:1 THF/pyridine (1.31 mL) was added 70% HF-pyridine (328 uL), affording a 0.0075M solution of 1:2:2 HF-pyr/THF/pyridine. The reaction mixture was allowed to warm to room temperature. After 20 h, H₂O (328 uL) was added, and the reaction mixture was heated to 40° C. After 2 h, the reaction mixture was cooled to 0° C., diluted with 50% EtOAc/Hex (5 mL), and slowly quenched by adding saturated aqueous NaHCO₃ dropwise until bubbling ceased. The layers were separated, and the aqueous layer was extracted with 80% EtOAc/Hex (5×5 mL). The combined organic layers were dried over NaSO₄, filtered, and concentrated. Purification was accomplished by silica gel flash column chromatography (25-50% EtOAc/Hex), followed by RP-HPLC (60-100% MeCN/H₂O) affording SUW208 (6.6 mg, 55% yield over 2 steps).

Characterization data for SUW208: TLC R_{f}=0.36 (60% EtOAc/Hex, UV active, stains in CAM); [α]²³_{D}=6.9° (c=0.10, CH₂Cl₂); IR (thin film): 3455 (bs), 2927, 1715, 1601, 1444, 1366, 1313, 1233, 1158, 1055 cm⁻¹; ¹H-NMR (500 MHz, CDCl₃) δ 7.42-7.32 (m, 3H, C₄₂H), 7.30 (t, J=7.9 Hz, 2H), 7.09-7.03 (m, 1H), 6.58 (t, J=11.3 Hz, 1H, C₄₁H), 6.55 (s, 1H), 6.09 (dt, J=14.7, 7.1 Hz, 1H, C₄₃H), 6.02 (d, J=1.9 Hz, 1H, C₃₄H), 5.80 (d, J=15.8 Hz, 1H, C₁₇H), 5.55 (d, J=11.3 Hz, 1H, C₄₀H), 5.28 (dd, J=15.8, 8.4 Hz, 1H, C₁₆H), 5.22 (s, 1H), 5.23-5.17 (m, 1H, C₂₅H), 5.19 (s, 1H), 5.15 (dd, J=11.7, 4.7 Hz, 1H, C₇H), 5.04-4.94 (m, 1H, C₁₃H), 4.33-4.12 (m, 4H, C₃—OH, C₅H, C₃H, C₁₅H), 4.08-4.00 (m, 1H, C₂₃H), 3.90-3.79 (m, 2H, C₁₁H, C₂₆H), 3.73-3.63 (m, 1H, C₂₂H_{eq}), 3.68 (s, 3H), 2.70 (bs, 1H, C₉—OH), 2.57-2.46 (m, 2H, C₂H₂), 2.04 (s, 3H, C₇—OAc), 1.12 (s, 3H), 1.01 (s, 3H), 0.99 (s, 3H), 0.94-0.90 (m, 6H); ¹³C-NMR (125 MHz, CDCl₃, 2 signals obscured by solvent) δ 172.6, 171.0, 167.2, 165.7, 152.0, 151.0, 146.5, 145.7, 139.4, 137.8, 129.2, 129.1, 128.5, 123.7, 119.8, 118.7, 101.9, 99.2, 74.1, 73.9, 73.0, 70.3, 69.4, 68.6, 65.9, 64.8, 51.3, 45.0, 42.5, 42.0, 41.1, 40.0, 39.7, 38.2, 35.9, 35.2, 33.5, 24.7, 22.0, 21.3, 21.1, 19.9, 19.8, 17.0, 13.8; HRMS calculated for C₅₁H₇₁NaNO₁₇ [M+Na]⁺: 992.4614; found 992.4585 (TOF ESI+).

Synthetic Procedure for SUW209

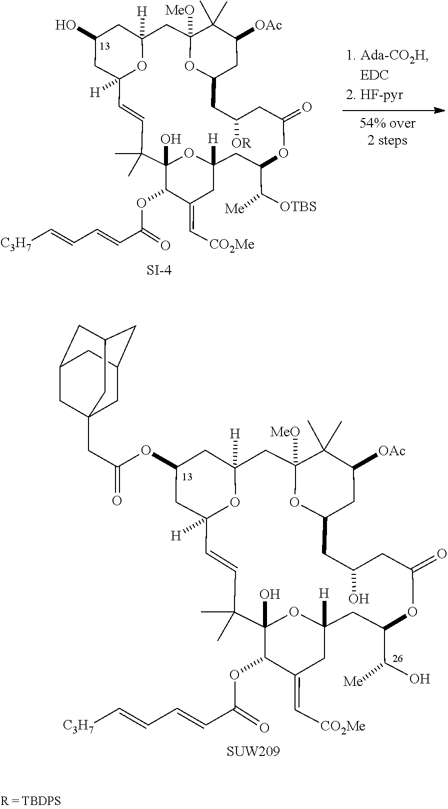

Acylation: To a solution of alcohol SI-4 (10 mg, 0.0082 mmol, 1 equiv) in CH₂Cl₂ (1 mL) was sequentially added 1-adamantaneacetic acid (8 mg, 0.041 mmol, 5 equiv), EDC-HCl (7.9 mg, 0.041 mmol, 5 equiv), and DMAP (1 crystal). After 20 h, TLC analysis indicated complete conversion of starting material. The reaction mixture was directly flashed via silica gel flash column chromatography (10% EtOAc/Hex) affording the C13-adamantyl ester in quantitative yield.

Global deprotection: To a cooled (0° C.) solution of this ester (1 equiv) in 1:1 THF/pyridine (1.5 mL) was added 70% HF-pyridine (375 uL), affording a 1:2:2 HF-pyr/THF/pyridine solution. The reaction mixture was allowed to warm to room temperature. After 24 h, H₂O (300 uL) was added, and the reaction mixture was heated to 30° C. After 4 h, the reaction mixture was cooled to 0° C., diluted with EtOAc (10 mL), and slowly quenched by adding saturated aqueous NaHCO₃ dropwise until bubbling ceased. The layers were separated, and the aqueous layer was extracted with 80% EtOAc/Hex (5×10 mL). The combined organic layers were dried over NaSO₄, filtered, and concentrated. Purification was accomplished by silica gel flash column chromatography (40-100% EtOAc/Hex), followed by RP-HPLC (70-100% MeCN/H₂O) affording SUW209 (4.5 mg, 54% yield).

Characterization data for SUW209: TLC R_{f}=0.33 (50% EtOAc/Hex, UV active, dark blue spot in p-anisaldehyde); [α]²⁴_{D}=12.5° (c=0.125, CH₂Cl₂); IR (thin film): 3461 (bs), 2906, 2849, 1720, 1640, 1366, 1245, 1158, 1137, 1100, 1058, 1003 cm⁻¹; ¹H-NMR (600 MHz, CDCl₃) δ 6.20-6.09 (m, 2H, C₄₂H, C₄₃H), 6.00 (bs, 1H, C₃₄H), 5.79 (d, J=15.6 Hz, 1H), 5.78 (d, J=15.6 Hz, 1H), 5.31-5.12 (m, 5H), 5.02-4.94 (m, 1H, C₁₃H), 4.31-4.10 (m, 4H), 4.07-3.98 (m, 1H), 3.86-3.78 (m, 2H), 3.73-3.64 (m, 1H, C₂₂H_{eq}), 3.67 (s, 3H), 2.04 (s, 3H, C₇—OAc), 2.02 (s, 2H), 1.97 (s, 3H), 1.59 (s, 6H), 1.57 (s, 6H), 1.24 (d, J=6.4 Hz, 3H, C₂₇H₃), 1.13 (s, 3H), 0.99 (s, 6H), 0.96-0.87 (m, 6H); ¹³C-NMR (125 MHz, CDCl₃, 1 signal obscured by solvent) δ 172.5, 171.3, 170.9, 167.1, 165.7, 152.1, 146.5, 145.7, 139.3, 129.1, 128.5, 119.8, 118.8, 102.0, 99.1, 74.1, 73.9, 72.9, 70.2, 69.5, 69.4, 68.6, 65.9, 64.8, 51.2, 49.3, 45.0, 42.6 (3C), 42.0, 41.1, 40.0, 39.6, 38.1, 36.9 (3C), 35.9, 35.2, 33.5, 33.0, 31.4, 28.7 (3C), 24.7, 22.2, 22.0, 21.3, 21.2, 19.9, 19.8, 17.0, 13.8; HRMS calculated for C₅₆H₈₂NaO₁₇ [M+Na]⁺: 1049.5444; found 1049.5413 (TOF ESI+).

Synthetic Procedure for SUW210

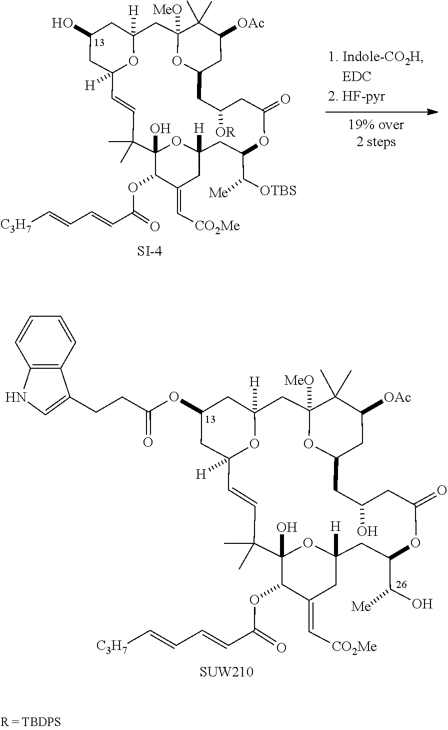

Acylation: To a solution of alcohol SI-4 (10 mg, 0.008 mmol, 1 equiv) in CH₂Cl₂ (0.5 mL) was sequentially added indole-3-propionic acid (8 mg, 0.041 mmol, 5 equiv), EDC-HCl (8 mg, 0.0041 mmol, 5 equiv), and DMAP (1 crystal). After 24 h, the reaction mixture was directly flashed via silica gel flash column chromatography (20% EtOAc/Hex) affording the C13-indole ester (assume quantitative yield).

Global deprotection: To a cooled (0° C.) solution of this ester (assume 0.008 mmol, 1 equiv) in 1:1 THF/pyridine (400 uL) was added 70% HF-pyridine (200 uL), affording a 1:2:2 HF-pyr/THF/pyridine solution. The reaction mixture was allowed to warm to room temperature. After 36 h, H₂O (200 uL) was added, and the reaction mixture was heated to 40° C. After 4 h, the reaction mixture was cooled to 0° C., diluted with EtOAc (10 mL), and slowly quenched by adding saturated aqueous NaHCO₃ dropwise until bubbling ceased. The layers were separated, and the aqueous layer was extracted with 80% EtOAc/Hex (5×10 mL). The combined organic layers were dried over NaSO₄, filtered, and concentrated. Purification was accomplished by silica gel flash column chromatography (50-80% EtOAc/Hex), followed by RP-HPLC (60-100% MeCN/H₂O) affording SUW210 (1.6 mg, 19% over 2 steps).

Characterization data for SUW210: TLC R_{f}=0.53 (75% EtOAc/Hex, UV active, purple spot in p-anisaldehyde); [α]²⁴_{D}=11.2° (c=0.085, CH₂Cl₂); IR (thin film): 3454 (bs), 2922, 2851, 1721, 1461, 1366, 1260, 1158, 1098, 1027 cm⁻¹; ¹H-NMR (600 MHz, CDCl₃) δ 7.96 (bs, 1H, NH), 7.59 (d, J=7.8 Hz, 1H), 7.35 (d, J=8.0 Hz, 1H), 7.18 (app. t, J=7.1 Hz, 1H), 7.11 (app. t, J=7.4 Hz, 1H), 7.00 (s, 1H), 6.21-6.11 (m, 2H, C₄₂H, C₄₃H), 6.00 (d, J=2.0 Hz, 1H, C₃₄H), 5.78 (app. t, J=15.1 Hz, 2H, C₄₀H, C₁₇H), 5.25 (dd, J=15.8, 8.4 Hz, 1H, C₁₆H), 5.24-5.17 (m, 1H), 5.20 (s, 1H, C₂₀NH), 5.15 (dd, J=11.8, 4.8 Hz, 1H), 5.01-4.93 (m, 1H, C₁₃H), 4.23 (t, J=11.8 Hz, 1H), 4.20-4.14 (m, 1H), 4.11 (t, J=10.0 Hz, 1H), 4.07-3.99 (m, 1H), 3.89-3.80 (m, 1H), 3.81-3.74 (m, 1H), 3.72-3.64 (m, 1H), 3.67 (s, 3H), 3.08 (t, J=7.5 Hz, 2H), 2.68 (t, J=7.6 Hz, 2H), 2.05 (s, 3H, C7-OAc), 1.12 (s, 3H), 0.99 (s, 6H), 0.94-0.90 (m, 6H); ¹³C-NMR (125 MHz, CDCl₃, 1 signal obscured by solvent) δ 173.0, 172.6, 171.0, 167.1, 165.7, 152.0, 146.6, 145.8, 139.2, 136.4, 129.1, 128.5, 127.3, 122.2, 121.6, 119.8, 119.5, 118.9, 118.7, 115.0, 111.2, 101.9, 99.2, 74.1, 73.9, 73.0, 70.3, 69.9, 69.3, 68.6, 65.9, 64.8, 51.3, 45.0, 42.5, 42.0, 41.1, 40.0, 39.3, 37.9, 35.9, 35.4, 35.2, 33.5, 31.4, 24.7, 22.0, 21.3, 21.1, 20.8, 19.9, 19.8, 17.0, 13.9; HRMS calculated for C₅₅H₇₅NaNO₁₇ [M+Na]⁺: 1044.4927; found 1044.4897 (TOF ESI+).

Synthetic Procedure for SUW211

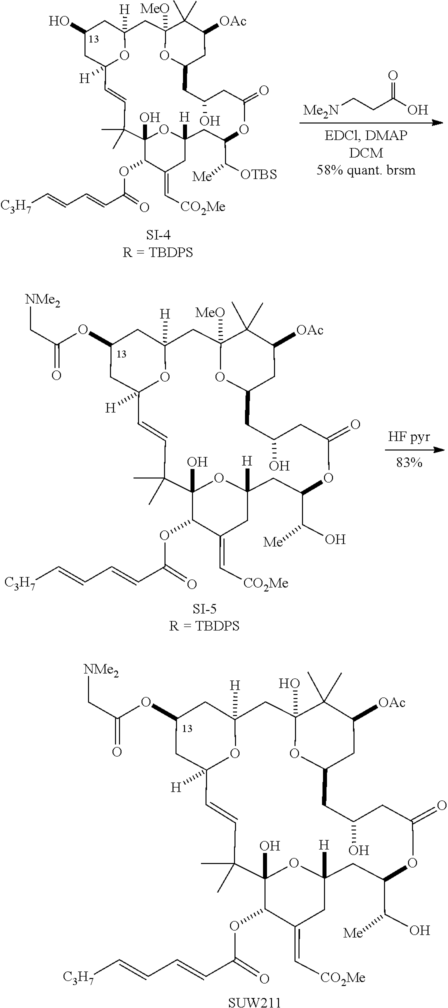

Acylation: To a vial containing SI-4 (16 mg, 0.016 mmol, 1 equiv) in DCM (0.5 mL) was added dimethylglycine (8.4 mg, 0.082 mmol, 5 equiv), EDCI (16 mg, 0.016 mmol, 5 equiv) and DMAP (10 mg, 0.082 mmol, 5 equiv). After stirring at room temperature for 16 h, the mixture was partitioned between DCM and saturated NaHCO₃. After extraction with DCM (2×), the combined organics were dried over Na2SO₄, filtered and concentrated. Flash chromatography (30-40% EtOAc/hexane) provided glycinate SI-5 as a white residue (10 mg, 58% yield, quant. brsm), which was carried forward to the next reaction.

Deprotection: Glycinate SI-5 was dissolved in 1:1 THF:pyridine (0.6 mL) in a polypropylene vial. HF:pyridine (0.2 mL) was added and the reaction mixture was stirred at 40° C. for 20 h, whereupon water (0.2 mL) was added and the resulting mixture stirred at the same temperature for 2.5 h. The reaction was quenched with sat. NaHCO₃, extracted with EtOAc (2×) and the combined organics dried over Na₂SO₄. Flash chromatography (5-10% MeOH/DCM) provided SUW211 as a white residue (6 mg, 83% yield).

Characterization data for SUW211: TLC R_{f}=0.5 (10% MeOH/DCM, UV active, purple spot in p-anisaldehyde); [α]^{22.7}_{D}=8.2° (c=0.23, CH₂Cl₂); IR (thin film): 3457, 3396, 3376, 2957, 2927, 1735, 1720, 1655, 1407, 1324, 1244, 1079, 1003 cm-1; ¹H NMR (600 MHz, CDCl₃) δ 7.39-7.30 (m, 1H), 6.14 (d, J=5.5 Hz, 2H), 5.98 (s, 1H), 5.76 (dd, J=15.5, 4.0 Hz, 2H), 5.27 (s, 1H), 5.24 (dd, J=15.8, 8.3 Hz, 1H), 5.17 (m, 3H), 5.11 (m, 2H), 4.26 (d, J=12.1 Hz, 1H), 4.12 (m, 1H), 4.00 (t, J=11.2 Hz, 1H), 3.89 (t, J=9.6 Hz, 1H), 3.80-3.76 (m, 1H), 3.69-3.59 (m, 4H), 3.46 (m, 1H), 2.67 (bs, 4H), 2.51-2.46 (m, 2H), 2.13 (m, 2H), 2.02 (m, 5H), 1.98 (d, J=13.2 Hz, 1H), 1.91 (t, J=13.0 Hz, 2H), 1.83 (m, 2H), 1.73 (m, 1H), 1.68 (d, J=15.1 Hz, 1H), 1.59 (m, 1H), 1.47-1.40 (m, 4H), 1.35 (d, J=11.7 Hz, 1H), 1.29-1.26 (m, 1H), 1.24-1.21 (m, 5H), 1.10 (s, 3H), 0.97 (m, 5H), 0.90 (m, 6H), 0.86 (t, J=7.1 Hz, 2H); ¹³C NMR (126 MHz, CDCl₃) δ 171.17, 167.27, 165.80, 152.16, 146.62, 145.82, 141.22, 139.52, 138.54, 129.19, 128.62, 119.89, 118.86, 102.13, 99.23, 74.21, 74.07, 73.13, 70.32, 69.41, 68.76, 65.93, 64.90, 51.35, 45.06, 44.44, 42.73, 42.03, 41.28, 40.13, 39.14, 37.97, 35.33, 34.39, 33.56, 31.53, 29.98, 24.85, 22.61, 22.13, 21.48, 21.32, 19.95, 19.79, 17.16, 14.34, 13.96; HRMS calculated for C₄₈H₇₃NO₁₇Na [M+Na]+: 936.4951; found 936.4950 (TOF ESI+).

Synthetic Procedure for SUW212

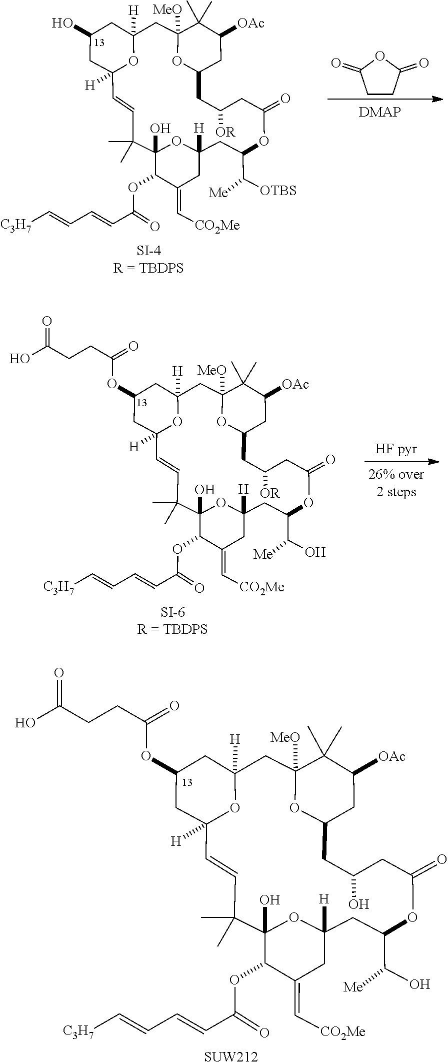

Acylation: To a vial charged with SI-4 (11 mg, 0.011 mmol, 1 equiv) in DCM (0.5 mL) was added succinic anhydride (3.4 mg, 0.034 mmol, 3 equiv) and DMAP (4.1 mg, 0.034 mmol, 3 equiv). After 16 h of stirring, the reaction was partitioned between DCM and saturated NH₄Cl. After extraction with DCM (2×), the combined organics were dried over Na2SO₄, filtered and concentrated. Flash chromatography (50% EtOAc/hexane) provided succinate SI-6 as a white residue (8 mg).

Deprotection: Succinate SI-6 was dissolved in 1:1 THF:pyridine (0.48 mL) in a polypropylene tube. HF-pyridine (0.12 mL) was added down the side of the tube. The reaction mixture was heated at 40° C. in an oil bath for 20 h, at which point H₂O (0.1 mL) was added. After an additional 2.5 h of stirring at 40 C, the reaction was partitioned between H₂O and EtOAc. Following extraction with EtOAc (3×), the combined organics were concentrated and purified by flash chromatography (10% MeOH/DCM) to provide SUW212 (2.7 mg, 26% over 2 steps).

Characterization data for SUW212: TLC R_{f}=0.6 (10% MeOH/DCM, UV-active, purple spot in p-anisaldehyde); [α]^{23.1}_{D}=3.0° (c=0.27, CH₂Cl₂); IR (thin film): 3461, 2956, 2927, 1734, 1717, 1636, 1617, 1559, 1364, 1245, 1159, 1100, 1002 cm-1; ¹H NMR (600 MHz, CDCl₃) δ 7.39-7.30 (m, 1H), 6.19-6.10 (m, 2H), 5.98 (d, J=2.0 Hz, 1H), 5.74 (dd, J=29.8, 15.5 Hz, 2H), 5.27 (s, 1H), 5.24 (dd, J=15.8, 8.3 Hz, 1H), 5.20-5.11 (m, 4H), 4.97 (td, J=11.1, 5.6 Hz, 1H), 4.33 (m, 1H), 4.20-4.10 (m, 2H), 4.07 (t, J=9.9 Hz, 1H), 4.01 (t, J=11.1 Hz, 1H), 3.95-3.89 (m, 1H), 3.78 (p, J=6.4 Hz, 1H), 3.64 (m, 4H), 2.75-2.51 (m, 4H), 2.44 (d, J=12.2 Hz, 1H₁), 2.13 (q, J=7.1 Hz, 2H), 2.08-1.94 (m, 7H), 1.90 (t, J=11.9 Hz, 1H₁), 1.86-1.77 (m, 3H), 1.75-1.69 (m, 1H), 1.67 (d, J=15.0 Hz, 1H), 1.60-1.52 (m, 1H), 1.49-1.40 (m, 3H), 1.35-1.25 (m, 3H), 1.24-1.20 (m, 6H), 1.10 (s, 3H), 0.98 (s, 3H), 0.96 (s, 3H), 0.94 (s, 3H), 0.89 (t, J=7.4 Hz, 3H), 0.86 (t, J=7.2 Hz, 2H); ¹³C NMR (126 MHz, CDCl₃) δ 171.74, 171.32, 167.33, 165.84, 152.27, 146.67, 146.63, 145.82, 129.63, 128.65, 119.88, 118.92, 102.17, 99.27, 95.83, 74.27, 74.21, 73.35, 70.23, 69.40, 68.81, 65.81, 64.91, 51.38, 46.59, 45.05, 42.63, 41.33, 40.22, 39.20, 38.13, 35.78, 35.35, 33.63, 31.57, 30.00, 28.86, 24.89, 22.59, 22.15, 21.52, 21.42, 20.04, 19.65, 18.15, 17.22, 13.98.

### Example 3: PKC Binding Assays

With a panel of compounds bearing diverse functionality at C13 in hand, we began to explore how these modifications affect biological function. Because a prerequisite for PKC pathway involvement is binding to PKC, compounds were initially evaluated for PKC affinity in a cell-free competitive binding assay with tritiated phorbol dibutyrate ([³H]-PDBu). Assays were performed with representative members of both the conventional (PKCα) and novel (PKCδ) PKC families. Subsequently, compounds were assayed for PKC activation in living cells using an isoform translocation assay. Translocation to the plasma membrane is the hallmark of PKC activation and therefore optically monitoring the subcellular localization of a PKCδ-GFP fusion protein via confocal microscopy can be used as an assay to determine whether a compound enters a cell and engages its PKC isoform target (Wender et al. Proc. Natl. Acad. Sci. U.S.A. 2011, 108 (17), 6721-6726).

PKC Binding Assay Protocol

The protein kinase C (PKC) affinity of bryostatin 1 and bryostatin analogs was performed via competition with ³H-phorbol-12,13-dibutyrate (³H-PDBu) as described below. This procedure entails a glass-fiber filtration method to determine bound radioligand.

Preparation of PKC Binding Assay Buffer

To a 50 mL polypropylene tube was added Tris-HCl (pH 7.4, 1 M, 1 mL), KCl (1 M, 2 mL), CaCl₂) (0.1 M, 30 μL), and bovine serum albumin (BSA, 40 mg, Sigma-Aldrich). This mixture was diluted to 20 mL with deionized H₂O and mixed gently. The buffer was stored on ice until use. The final concentration of these constituents is shown in Table 1:

TABLE 1
PKC binding assay buffer composition

| | Stock | | | Final | |
|---|---|---|---|---|---|
| Constituent | concentration | Quantity | | Concentration | |
| pH 7.4 | 1.0M | 1.0 | mL | 50 | mM |
| Tris-HCl | | | | | |
| KCl | 1.0M | 2.0 | mL | 100 | mM |
| CaCl₂ | 0.10M | 30 | μL | 0.15 | mM |
| BSA | — | 40 | mg | 2 | mg/mL |

| | | | |
|---|---|---|---|
| Deionized | — | Final vol of | — |
| H₂O | | 20 mL | |

Preparation of Phosphatidylserine (PS) Vesicles

For every two assays, 3.5 mg phosphatidylserine (Avanti Polar Lipids, porcine, 25 mg/mL CHCl₃ solution) was concentrated by removing chloroform under a stream of nitrogen followed by reduced pressure. The solid PS was suspended as vesicles in freshly prepared PKC binding assay buffer (3.5 mL) by sonicating six times for 30 sec, with a 30 sec rest between sonications (Branson Sonifier 250, power=2, 50% duty cycle). The resulting milky cloudy mixture (1 mg/mL) was stored on ice until use.

Preparation of PKC Isoform Solution

Assay PKC was prepared by dissolving a 4 μg aliquot of the indicated recombinant human PKC isoform (Invitrogen) into 11.6 mL of PKC binding assay buffer (this amount is sufficient for two assays). The diluted PKC was stored on ice for immediate use.

Preparation of ³H-PDBu Solution

³H-PDBu (American Radiolabeled Chemicals, Inc.; 1 mCi/mL acetone solution; specific activity: 20 Ci/mmol) was diluted 10-fold with DMSO. The resulting 500 nM stock solution was further diluted with DMSO to 30 nM.

Preparation of Analog Compound Dilutions

Compound dilutions were prepared by serially diluting from a chosen “high” concentration by factors of 3 or 4. For each analog compound, seven concentrations were used to define the inhibition curve (i.e. for SUW200, the analog concentrations used were 3000 nM, 750 nM, 188 nM, 46.9 nM, 11.7 nM, 2.93 nM, and 0.73 nM).

“Master Mix” Solution

To a polypropylene tube was added 3.3 mL of 1 mg/mL PS vesicles solution, 11 mL of PKC isoform solution, and 1.1 mL of 30 nM ³H-PDBu solution were added. The resulting solution was vortexed to mix and stored on ice.

PKC Binding Assay Protocol

Materials:
Glass-fiber filters (Whatman GF/B) were prepared by soaking them in a solution of aqueous polyethyleneimine (10% by vol, 18 mL) in deionized water (600 mL) for ≥1 h.
500 mL “rinsing buffer” of 20 mM Tris, pH 7.4 was cooled on ice for the duration of the incubation period and for the remainder of the assay.

Triplicate data points were obtained for each analog concentration. For each data point, 280 μL of “Master Mix” Solution and 20 μL of analog compound at a specified concentration were added to a polypropylene tube. Non-specific ³H-PDBu binding was assessed in triplicate by substitution of the analog compound with unlabeled PDBu (20 μL of a 75 μM stock, assay concentration: 5 μM). Maximal ³H-PDBu binding was assessed in triplicate by substitution of the analog compound with 20 μL DMSO. The solutions were vortexed to mix, incubated at 37° C. for 10 min, and incubated on ice for at least 30 min prior to filtration. Using a Brandel Harvester, the assay contents from each polypropylene tube were vacuum-filtered through polyethylenimine-soaked filters, washing with rinsing buffer (3×) and drying first under vacuum for 5 min and then under ambient conditions for ≥2 h. The resulting filters had circular perforations for each data point, which were removed with forceps and placed in a scintillation vial. Scintillation vials were filled with Bio-Safe II scintillation fluid (5 mL) and measured for radioactivity using a Beckman LS 6000SC scintillation counter. Counts per minute (cpm) were averaged for each triplicate dilution. The data were plotted—cpm vs. log(concentration)—using Prism® by GraphPad Software and an IC₅₀ was determined using that program's built-in one-site competition least squares regression function. Kᵢ values were calculated using the equation: Kᵢ=IC₅₀/(1+([3H−PDBu]/K_{d})). The K_{d} of ³H-PDBu for PKC isoforms was measured separately via saturation binding experiments under identical conditions.

PKCδ-GFP Translocation Assay Protocol

Cell Culture

Chinese hamster ovary factor K1 (CHO-k1, ATCC) cells were cultured in F-12 Kaighn's media (Hyclone, 10% fetal bovine serum, 1% penicillin/streptomycin added, referred to as F-12+/+ below) at 37° C. in an incubator (5% CO₂). Cell cultures were maintained by splitting cells 1:3 when they reached ˜75-100% confluence (every 2-3 days) as follows:

Media was aspirated (taking care not to disturb adherent cells) and 3 mL of 0.25% trypsin EDTA (Gibco) was used to remove the cells from the culture flask (T75, Falcon). 1 mL of the cell suspension was then added to 9 mL of fresh F-12+/+ and the sample was sub-cultured until reaching confluence (˜2-3 days).

Cell Plating

A confluent culture of CHO-k1 cells was detached from a T75 flask with 3 mL 0.25% trypsin EDTA. Cells were counted using a Countess II Automated Cell Counter (Fisher). The cell suspension was diluted to 240,000 cells/mL with fresh F-12+/+ and 2.5 mL of this diluted stock was added to one well in a 6-well plate. The cells were then cultured for 24 hours.

Transfection

Cells were transfected with Lipofectamine 2000 reagent (Invitrogen) or DA 13:11 at a 10:1 charge ratio as previously described by McKinlay et al. (PNAS 2017, 114, E448-E456).

Lipofectamine 2000

F-12+/+ was aspirated and cells were washed with F-12−/−. 2 mL of fresh F-12−/− was then added to each well, taking care not to disturb adherent cells. For each well of CHO-k1 cells, 12.5 μL Lipofectamine 2000 reagent (Invitrogen) was added to 250 μL Opti-MEM reduced serum media (Invitrogen) in a polypropylene tube and incubated for 20 minutes at RT. Meanwhile, for each well, 4 μg of PKCδ-GFP pDNA and 250 μL Opti-MEM reduced serum media was added to a separate polypropylene tube. 250 μL of the Lipofectamine 2000 suspension was added to the DNA suspension and the solution incubated for 30 minutes at RT. 500 μL of the Lipofectamine/DNA suspension was added to the respective wells of the 6-well plate. The cells were then incubated at 37° C. (5% CO2) for ˜24 hrs.

DA 13:11

F-12+/+ was aspirated and cells were washed with F-12−/−. 2.4 mL of fresh F-12−/− was then added to each well, taking care not to disturb adherent cells. A 4 μg aliquot of PKCδ-GFP pDNA was added to PBS (pH 5.5, final volume 100 μL). 5.6 μL of DA 13:11 (2 mM stock in DMSO) was then added to the DNA solution, and the mixture was gently mixed (by flicking) for 20 seconds, at which point it was added directly to the respective wells of the 6-well plate. The cells were then incubated at 37° C. (5% C02) for ˜24 hrs.

Plating on Chambered Coverglass Slides

After incubation, the media was aspirated and cells were washed with PBS (2.0 mL) and trypsinized (500 μL). The cell suspension was then diluted with 2.0 mL F-12+/+. 200 μL aliquots were added to 3 wells of a Lab-Tek II 4-well chambered coverglass slide (Fisher), producing 4 slides in total, each with three wells of cells. The cell suspension was directly diluted with 600 μL of additional F-12+/+. The resulting samples were incubated for ˜24 hrs. prior to imaging.

Dosing an Acquiring Data

Fluorescent images were obtained using a Leica SP8 White Light Confocal microscope and the Leica AF software package. Prior to analysis, media was aspirated and 800 μL of PBS (Hyclone, without Ca²⁺ or Mg²⁺) supplemented with glucose (10 mM) was added to each well of the chambered coverglass slide. Bryostatin and bryostatin analogs were diluted to the appropriate concentration in 200 μL of 10 mM glucose in PBS. Cells were located for imaging and data was recorded for three wells in parallel, imaging at predetermined positions in each well using adaptive focus control. Cells were imaged at 30 second intervals following the addition of compound (set to t=0) for 20-40 minutes. Data were recorded at room temperature. Images were exported as .lif files and fluorescence intensity was analyzed using FIJI (NIH) software. To monitor the translocation, small cytosolic regions of interest were selected in each cell, and fluorescence intensity values were plotted vs. time following background subtraction and normalization. Graphed data represents the average of at least replicates.

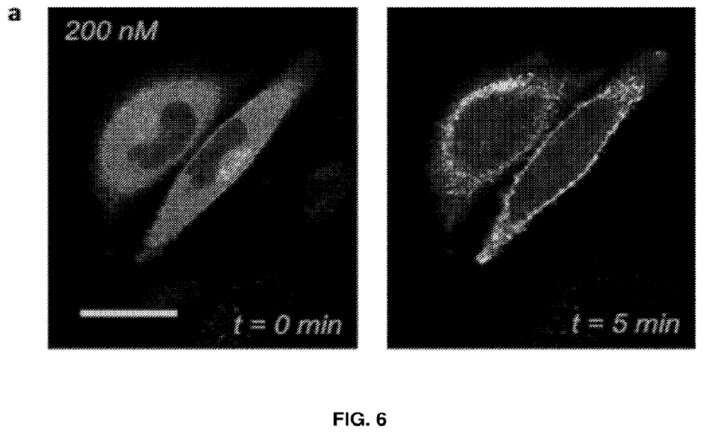

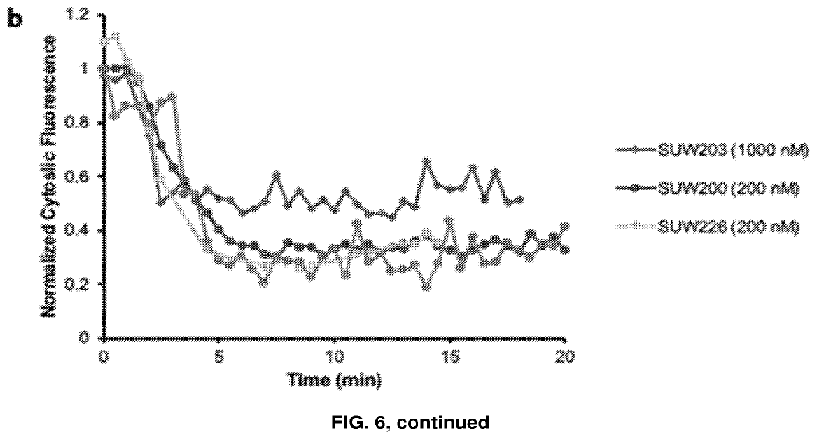

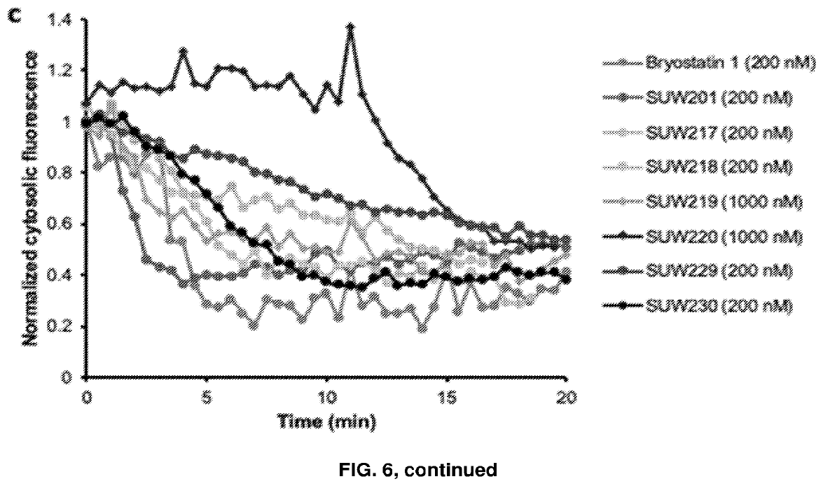

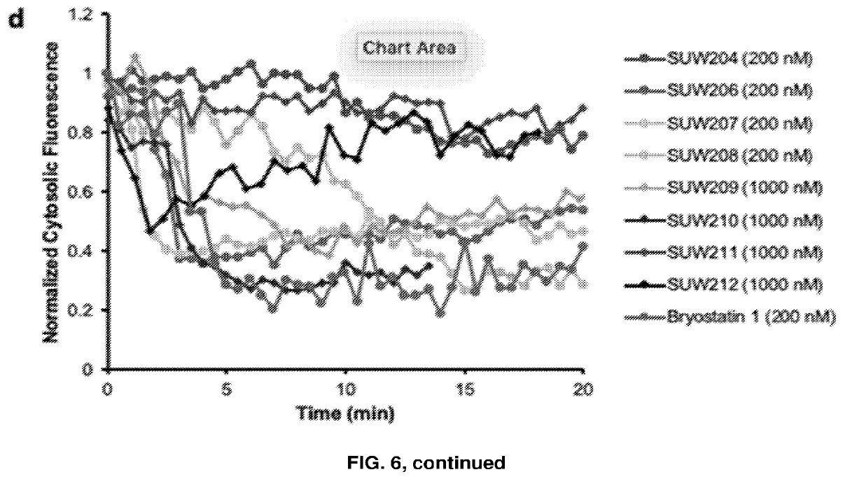

This array of biological assays provided key insights in to the effect of B-ring substitutions on compound function. Data are summarized in FIG. 6, panels A-B and Table 2.

| | Kᵢ | Kᵢ | % | Fold increase |
|---|---|---|---|---|
| | PKCα | PKCδ | Translocation | CD22 |
| Compound | (nM) | (nM) | (concentration*) | expression** |
| Bryostatin 1 | 0.8 | 1.1 | 70% (200 nM) | 2.10 |
| SUW200 | 4.1 | 7.6 | 70% (200 nM) | 1.73 |
| SUW201 | 0.54 | 1.4 | 65% (200 nM) | 1.59 |
| SUW203 | 1.0 | 4.1 | 50% (1000 nM) | 1.16 |
| SUW204 | 1.4 | 3.7 | 25% (200 nM) | 0.98 |
| SUW206 | 1.0 | 1.3 | 60% (200 nM) | 1.63 |
| SUW207 | 2.0 | 5.4 | 60% (200 nM) | 1.33 |
| SUW208 | 1.0 | 4.7 | 75% (200 nM) | 1.68 |
| SUW209 | 1.6 | 3.5 | 60% (1000 nM) | 1.45 |
| SUW210 | 5.6 | 7.3 | 75% (1000 nM) | 1.49 |
| SUW211 | 98 | 27 | 20% (1000 nM) | ND |
| SUW212 | 40 | 24 | 50% (1000 | ND |
| | | | nM)*** | |
| SUW217 | 9.2 | 9.4 | 75% (200 nM) | 1.97 |
| SUW218 | 7.1 | 6.6 | 55% (200 nM) | 1.92 |
| SUW219 | 37 | 25 | 60% (1000 nM) | ND |
| SUW220 | 16 | 15 | 50% (1000 nM) | ND |
| SUW226 | 5.8 | 6.6 | 75% (200 nM) | 1.76 |
| SUW229 | 1.8 | 1.2 | 50%(200 nM) | 2.35 |
| SUW230 | 4.2 | 9.4 | 65% (200 nM) | 1.62 |

As seen in Table 2, compounds were evaluated for PKC binding affinity in a competitive binding assay with [3H]-phorbol dibutyrate in PKCα and PKCδ, members of the conventional and novel PKC isoform families respectively. Cell entry and compound association with PKC informs in living cells in vitro was determined by monitoring translocation of a PKC-GFP fusion protein from the cytosol to the plasma membrane. Representative images are shown in FIG. 6, panel A. *Indicates minimum effective concentration required to induce translocation of PKC-GFP to the membrane. **At 1 nM, relative to DMSO control. ***Indicates only brief translocation observed (see FIG. 6, panel D). ND=not determined.

FIG. 6, panel A. Bryolog-induced activation of PKC determined by monitoring PKCδ-GFP translocation to the plasma membrane using confocal microscopy. FIG. 6, panels B-D. Cytosolic fluorescence normalized to t=0 (time immediately prior to addition of compound to media) and plotted against time. Error bars excluded for clarity. Maximum translocation of PKCδ-GFP to the plasma membrane reported in Table 2.

The goal was to determine whether cell-free PKC affinity and intracellular PKC translocation correlate and how binding and translocation influence downstream function (e.g., CD22 induction). As expected, nearly all of the compounds designed using our pharmacophore model retained potent binding affinity for PKC (<10 nm) comparable to the reported values of bryostatin 1 (Table 2). However, certain C13 functionalities decreased PKC binding affinity. For example, charged substituents at C13 (SUW211, SUW212) decreased affinity to PKC by ˜20-100-fold (Table 2), consistent with the less effective partitioning of these groups in the phosphatidyl serine (PS) vesicle complex (membrane surrogate used in the cell-free binding assay). Additionally, substitution of the C13 methyl-(Z)-enoate with a benzyl-(Z)-enoate resulted in a ˜30-fold decrease in potency. Similar decreases in binding affinity were not observed in C13 esters with more conformationally flexible linkers bearing large relatively hydrophobic substituents (SUW209, SUW210, Table 2). Aside from these exceptions, the majority of compounds tested exhibited single-digit nanomolar binding affinities to PKC, further validating the predictive value of our proposed pharmacophore model and prompting the progression of these compounds to more advanced in vitro PKC translocation assays.

In its inactive state, PKC resides in the cytosol. In contrast, upon binding to its endogenous ligand DAG or exogenous small molecule ligands such as the phorbol esters and bryostatin, the resulting PKC-ligand complex resides at the inner leaf of the plasma membrane (Newton, A. C. AJP Endocrinol. Metab. 2010, 298 (3), E395-E402). This translocation of PKC is a pre-requisite for PKC activation, and thus most downstream activities. Both experimental studies and molecular dynamics simulations suggest that the PKC-ligand complex can assume multiple bound states, putatively influencing the differential association of scaffolding proteins and phosphorylation of downstream effector proteins, thereby resulting in divergent signaling outcomes (Newton et al. Crit. Rev. Biochem. Mol. Biol. 2018, 53 (2), 208-230; Ryckbosch et al. Nat. Commun. 2017, 8 (1), 6; Newton, A. C. J. Biol. Chem. 1995, 270 (48), 28495-28498; and Newton, A. C. Chem. Rev. 2001, 101 (8), 2353-2364). This dynamic nature of the PKC signaling synapse allows PKC to transmit a variety of signals with diverse biological implications (Newton, A. C. AJP Endocrinol. Metab. 2010, 298 (3), E395-E402). Therefore, our goal was to develop compounds with modifications to the B-ring of the bryostatin scaffold that elicit differential signaling outcomes by establishing different interactions of the active signaling complex with the plasma membrane. Monitoring the membrane translocation of a PKCδ-GFP fusion in living cells is a convenient in vitro assay for compound function and cell permeability that also allows us to determine qualitative differences in on-target compound activity in real time (Wender et al. Proc. Natl. Acad. Sci. U.S.A. 2011, 108 (17), 6721-6726). Our design strategy for exemplary bryostatin analogs (vide supra) enabled us to examine how substituents at this position influence the dynamics of PKC activation.

Studies herein show that the thermodynamics of PKC binding and the kinetics of ligand cell entry and PKC translocation are often decoupled, a finding that could be exploited to control isoform-selective signaling. As a positive comparator, bryostatin 1, a single digit nanomolar binder of all PKC isoforms, translocates ˜70% of cytosolic PKCδ-GFP to the plasma membrane within 5 minutes at a concentration of 200 nM (Table 2, FIG. 6, panel C). While introducing hydrophilic substituents at C13 (SUW203, SUW204) generated compounds that bind both conventional and novel PKC isoforms with single-digit nanomolar affinity, these analogs exhibited different behavior than bryostatin 1 in the translocation assay (FIG. 6, panels B-C). At 200 nM SUW204 translocated ˜25% of cytosolic PKC to the plasma membrane at 20 minutes while SUW203 required a concentration of 1000 nM to translocate ˜50% of cytosolic PKC. Similarly, compounds with charged substituents at C13 (SUW211, SUW212) did not achieve sustained translocation of PKC at up to 1000 nM (Table 2, FIG. 6, panel D). This suggests that interaction of the membrane with the functionality on bryostatin's B ring is important for compound function and that PKC binding is necessary, but not sufficient for compound activity in vitro. Compounds with hydrophilic or charged modifications at C13 would putatively not be able to effectively embed in the plasma membrane, and thus would exhibit attenuated PKC function.

In addition to exploring the effect of hydrophilic functionality at C13, we also examined a series of C13 esters of different sizes, hydrophobicities, and varied structural motifs (aryl, heteroaryl, alkyl, adamantyl), all of which could be efficiently accessed from the corresponding C13 alcohol (see e.g., Scheme 3). While we found that diverse functionality was tolerated at this position with respect to PKC binding affinity, our analog library exhibited vastly different profiles in functional assays. In contrast to the free alcohol SUW204, capping the C13 hydroxyl group with smaller hydrophobic substituents generated compounds that were comparably effective to bryostatin 1 at translocating PKC (FIG. 6, panel D). The two diastereomers of the C13 acetate (SUW206 and SUW207) exhibited almost identical behavior both to each other and to bryostatin 1. While C13 phenyl carbamate SUW208 effectively translocated PKC to the plasma membrane at 200 nM, it exhibited a delayed time course, requiring about 15 minutes for translocation, relative to other analogs that were active in this assay (FIG. 6, panel D), indicating that small changes in size and/or polarity at this position can influence ligand-mediated PKC activation and signaling and/or the kinetics and efficacy of cell entry.

To complement the synthesis of compounds with relatively similar size requirements at C13, we also installed larger substituents at this position with vastly different functionalities than is found in the natural product. C13 adamantyl ester SUW209 and C13 indoyl ester SUW210 were designed to enhance potential membrane interactions via increased localized hydrophobicity and the potential to pick up cation-pi contacts with cationic lipid headgroups on the inner leaflet of the plasma membrane, respectively. We hypothesized that these two modes of membrane association could differentially bias the orientation of the PKC-ligand complex in the membrane and therefore affect downstream signaling outcomes. We found that while both compounds are high affinity PKC binders (Table 2), they are inactive at 200 nM in the PKC translocation assay and require 1000 nM to translocate ˜70% of cytosolic PKC to the plasma membrane at about 5 minutes (FIG. 6, panel D).

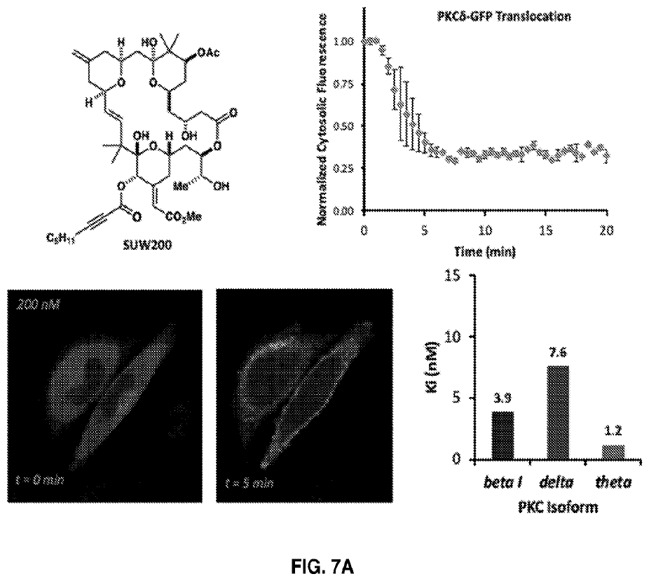

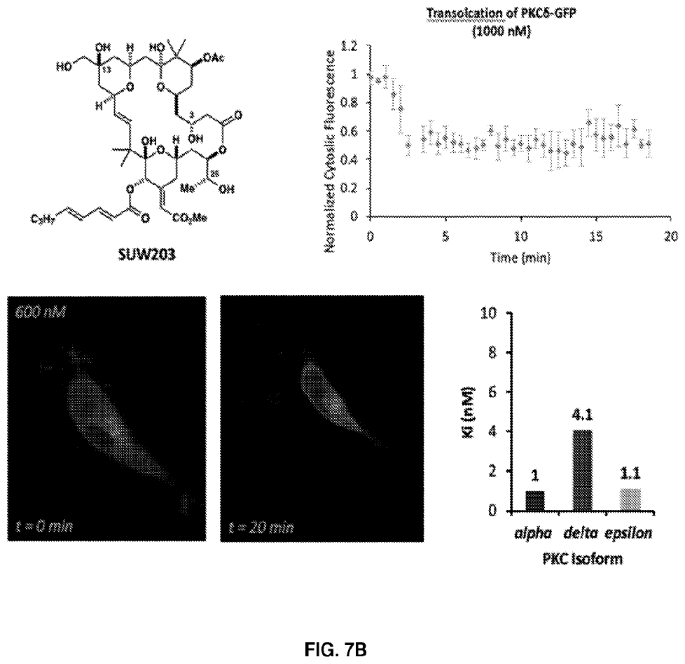

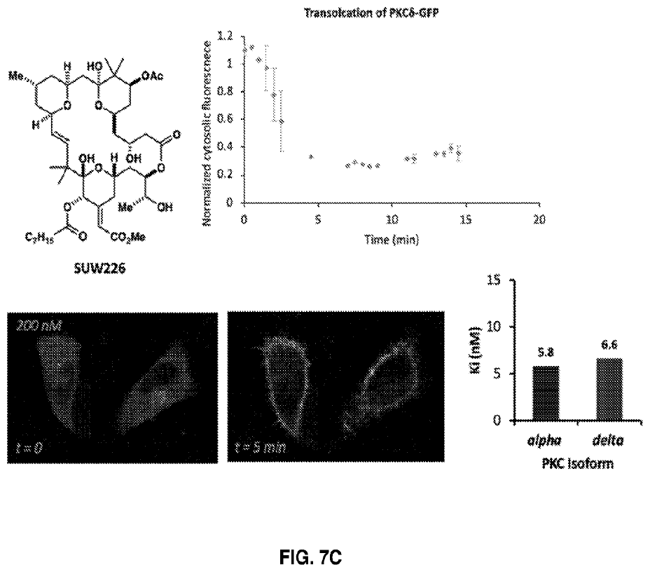

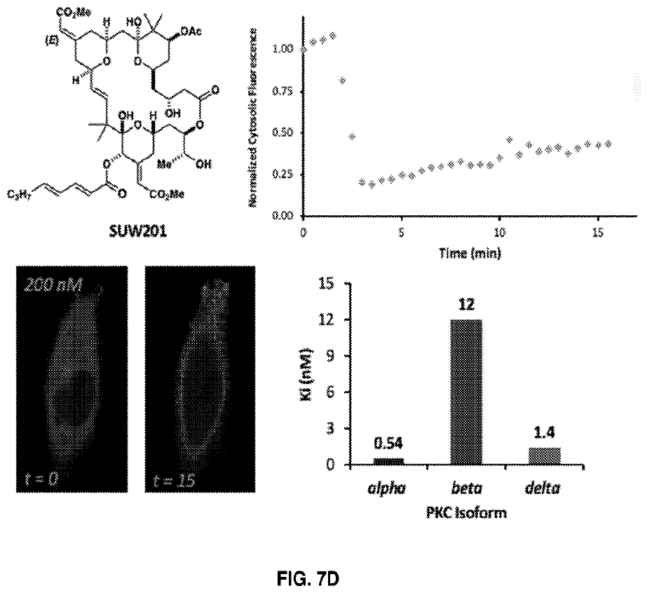

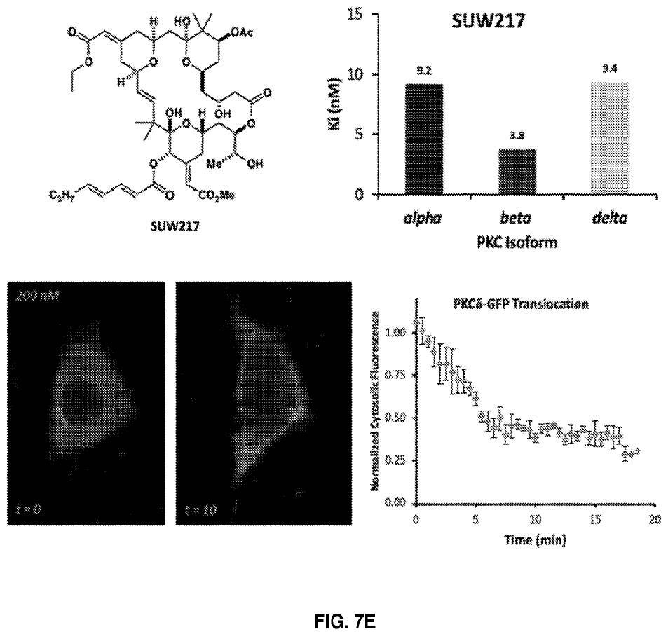

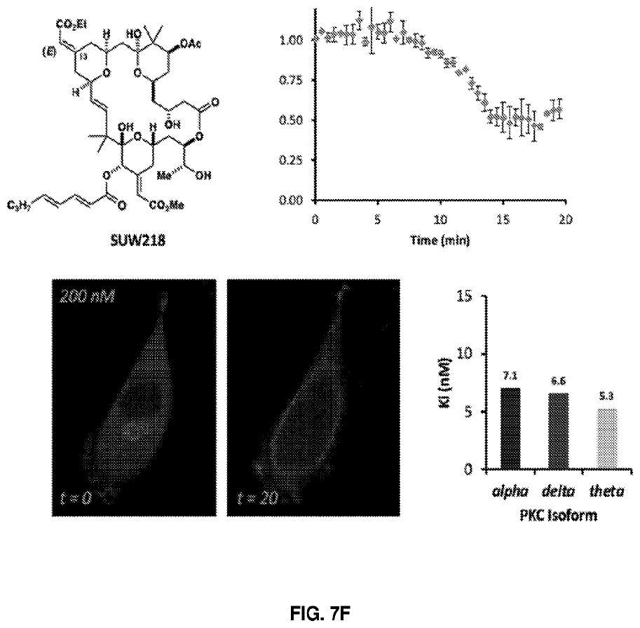

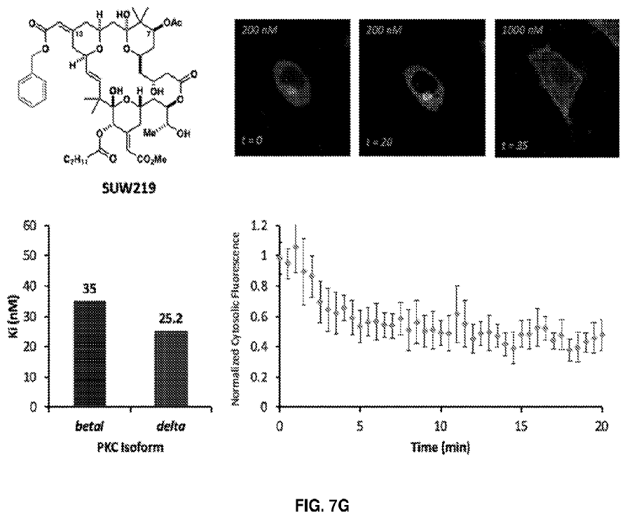

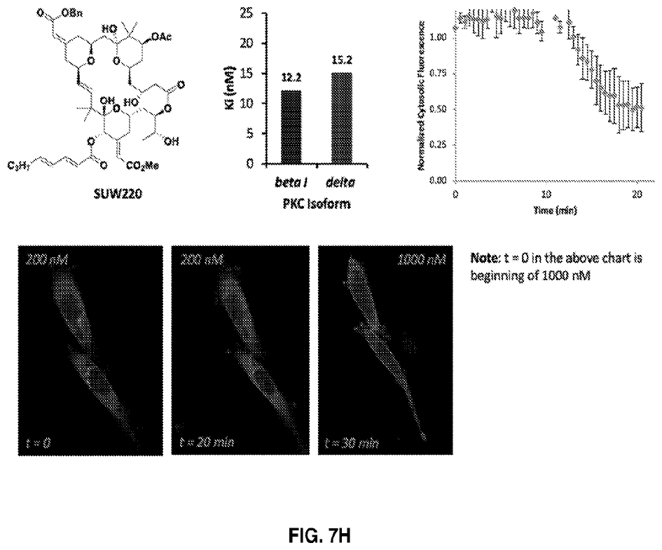

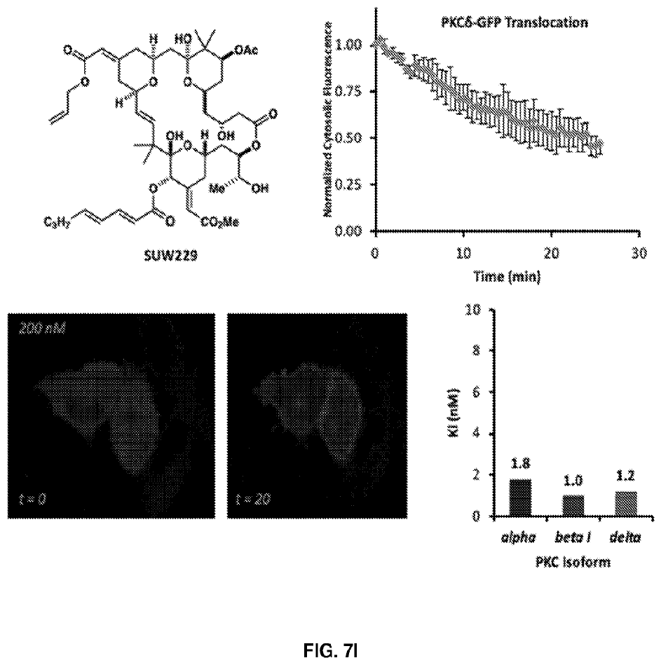

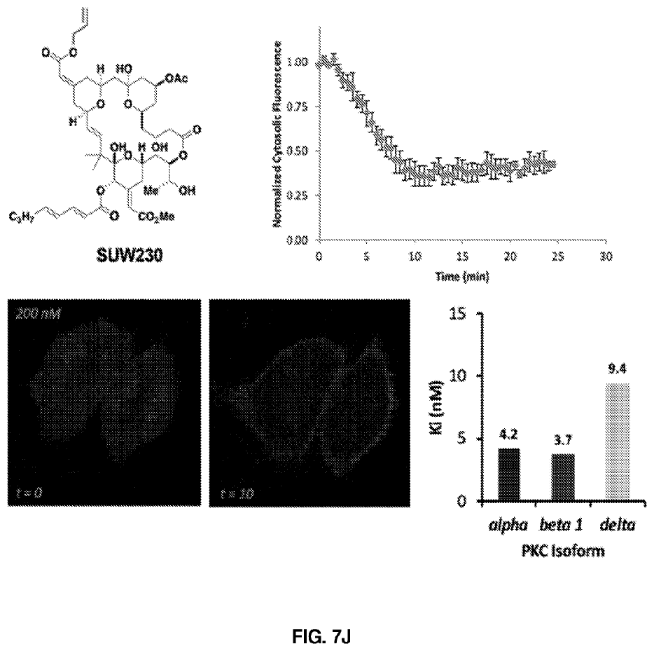

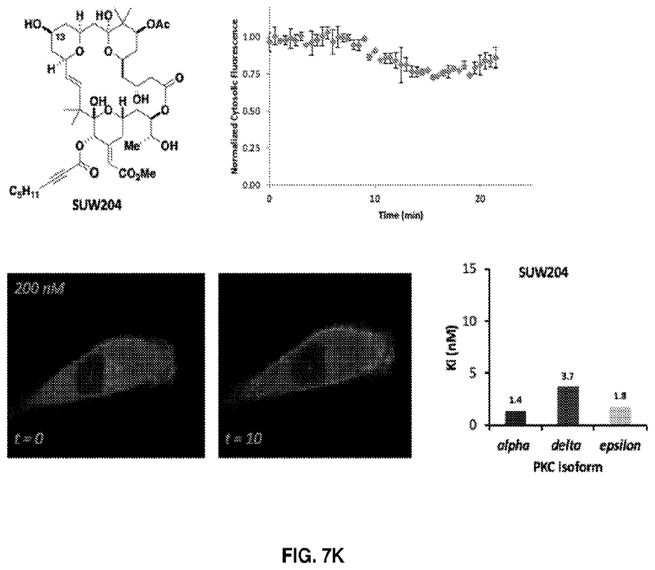

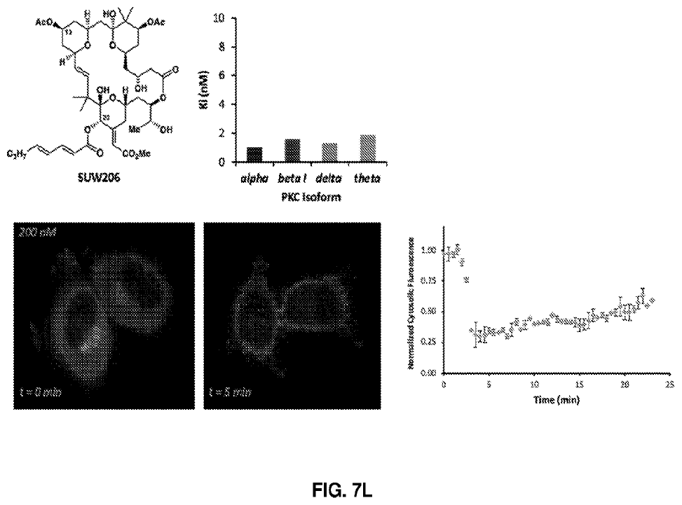

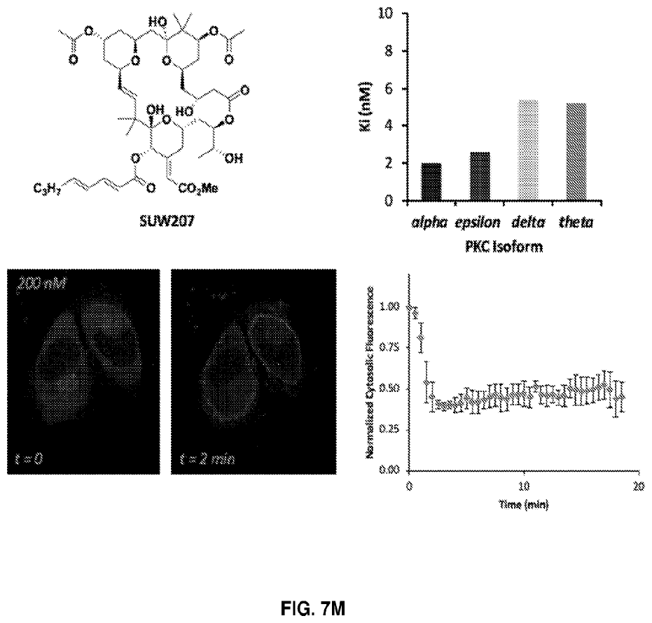

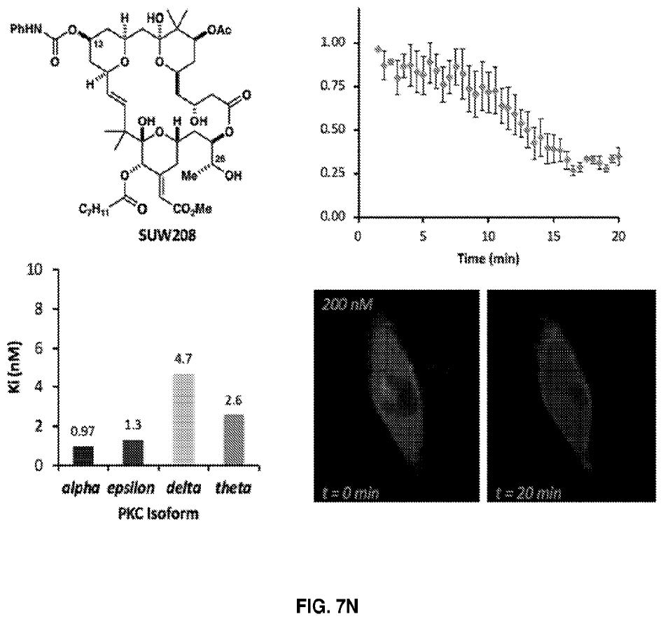

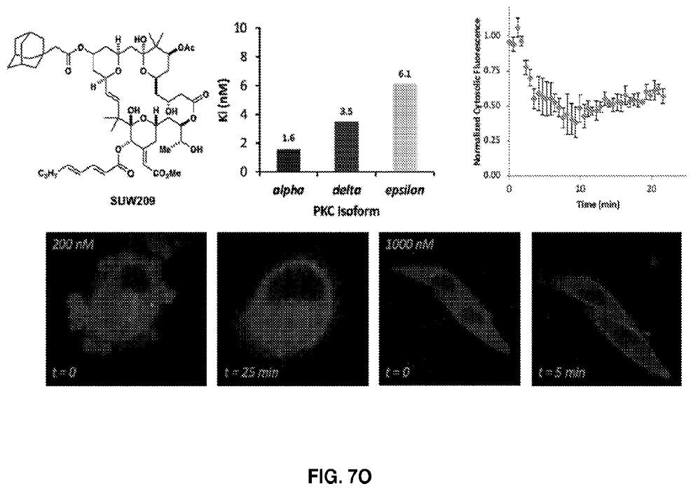

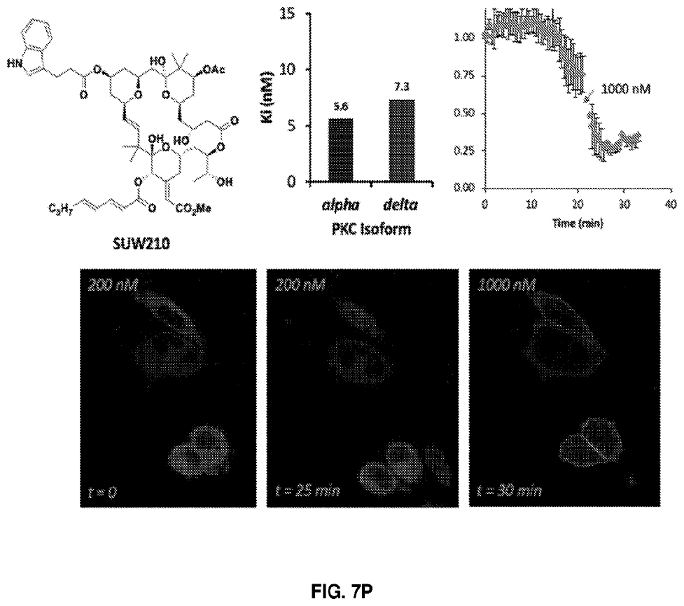

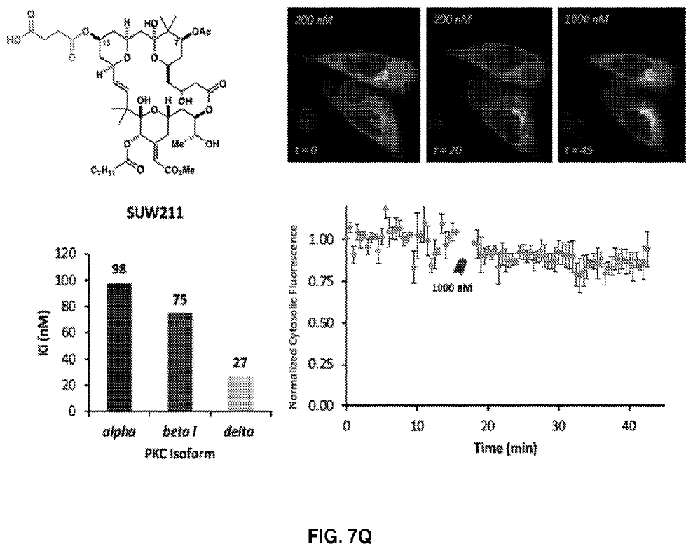

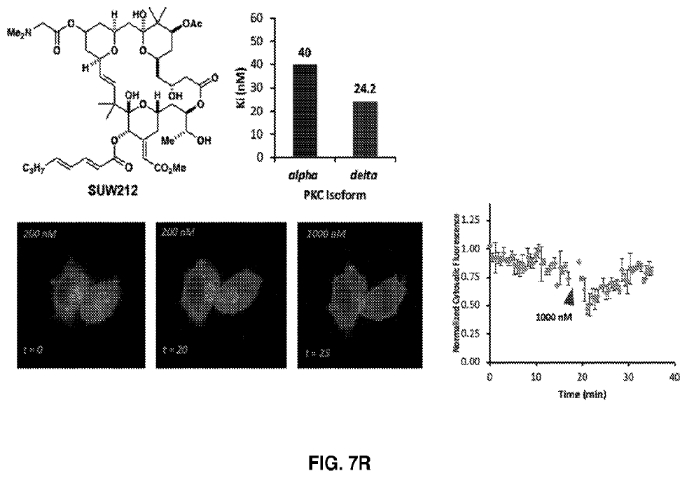

Next how C13 functionality can influence bulk compound properties, and the correlation between compound hydrophobicity (measured using c Log P) and activity (PKC translocation at 200 nM) was examined. Plotting c Log P vs. percentage of membrane associated PKCδ-GFP at 200 nM (FIGS. 7A and 7B) revealed an effective window of c Log P values required for efficient translocation. Compounds with c Log P values between 1.00 and 4.00 were active at 200 nM, suggesting that lipophilicity must be effectively balanced in considering modifications to the B ring of the bryostatin scaffold. It is also interesting to note that among compounds within the effective range of lipophilicity, variable dynamics of PKC activation were observed. While most compounds exhibit logarithmic activation curves, select compounds exhibit delayed activation patterns (SUW208, FIG. 6, panel D), or more sustained linear activation dynamics (SUW218, SUW229, FIG. 6, panel C). These observations of the kinetics of PKC ligand association and intracellular distribution of the complex can be rationalized by the timing of ligand entry into the cell and post-entry ligand-controlled partitioning of the PKC-ligand complex between the plasma membrane and cytosol, with more polar compounds having slower entry and a limited ability to form a stable complex with the plasma membrane. This is of potential consequence as it is well known that the dynamics of PKC activation can have profound consequences for downstream signaling outcomes (Alfonso, S. I. et al., Sci. Signal. 9, ra47, https://doi.org/10.1126/scisignal.aaf6209 (2016); Newton, A. C., AJP Endocrinol. Metab. 298, E395-E402 (2010)).

C13 alkyl enoates in both the (Z) and (E) geometries were well tolerated with the exception of benzyl enoates SUW219 and SUW220. Interestingly, benzyl enoate SUW219 binds PKC with a ˜2-fold decrease in affinity, suggesting that perhaps the (Z) olefin geometry can position C13 substituents in an orientation that impacts the conformation of pharmacophoric elements in the southern hemisphere. In general however, smaller, linear alkyl enoates are potent binders of PKC and active ligands in vitro (Table 2, FIG. 6, panel C). The C13 allyl enoates are of potential synthetic interest as this monosubstituted alkene could be used for final-step diversification of bryostatin at this position via olefin cross metathesis reactions (e.g., see Scheme 2).

### Example 4: CD22 Surface Expression Assay

Finally, we also determined the effect of compounds on CD22 expression in vitro in NALM6 cells, an ALL cell line previously studied in connection with CD22-targeted CAR T cell therapy (Fry et al. Nat. Med. 2018, 24 (1), 20-28; and Ramakrishna et al. Blood 2017, 130 (Suppl 1)). Having established the generally high affinities of members of exemplary bryostatin analogs and their PKC translocation kinetics and extentdynamics, we next sought to evaluate exemplary analogs in an assay pertinent to the clinical use of bryostatin and its analogs as adjuvants to enhance targeted cancer immunotherapies. CD22-targeted CAR T cell therapy has been reported to cure patients with ALL while those who fail this treatment are thought to have a lower surface density of CD22. Fry et al. conclusively demonstrated that a critical threshold of CD22 surface density is required for activation of anti-CD22 CAR T cells in vitro (Nguyen, S. et al., J. Clin. Oncol. 34, 10536-10536 (2016)) and tumor clearance in a murine tumor xenograft model (Fry et al. Nat. Med. 2017). In order to investigate bryostatin 1 and bryostatin analogs as adjuvant leads for CD22-targeted CAR T therapy, we developed an in vitro model for bryostatin-induced increased CD22 surface expression in ALL using NALM6 cells.

CD22 Surface Expression Protocol

Cell Culture

NALM6, clone G5 cells (ATCC) were cultured RPMI-1640 (Hyclone, +L-glutamine, +10 mM HEPES, 10% fetal bovine serum added, 1% penicillin/streptomycin added, referred to as RPMI-1640 unless otherwise noted below) at 37° C. in an incubator (5% CO₂). Cell cultures were maintained between 4×10⁵ and 3×10⁶ cells/mL according to vendor instructions.

Plating and Dosing

Cell suspension from a confluent T75 flask (Fisher) was transferred to a 15 mL falcon tube and centrifuged at 1100 rpm for 7 minutes. The supernatant was aspirated, and the cell pellet was resuspended in ˜5 mL of fresh RPMI-1640. Cells were counted using a Countess II Automated Cell Counter (Fisher). A 5.2 mL stock of 1×10⁶ cells/mL was prepared by diluting an aliquot of the cell suspension with additional RPMI-1640. 199 μL of this stock was added to 24 wells (enough for triplicate measurements for 8 different experimental conditions) in a 96-well plate. Dosing was performed in triplicate for DMSO, bryostatin 1, and bryostatin analogs. DMSO (negative control), 10 nM bryostatin 1 (positive control), and untreated samples (negative control) were included in each experiment. 1 μL of DMSO or the appropriate stock solution of compound in DMSO was added to each well. Cells were incubated for 24 hours, at which point the cell suspensions were transferred to 1.5 mL Eppendorf tubes and diluted with 1.0 mL of PBS (Hyclone, without Ca²⁺ or Mg²⁺). Samples were centrifuged at 2000 rpm for 5 min at RT. The supernatant was aspirated, and cell pellets were resuspended in 400-600 μL RPMI-1640. Cells were sub-cultured between 2×10⁵ and 3×10⁶ cells/mL in 24-well plates for an additional 24 hrs-7 days, at which point CD22 surface expression was assayed by flow cytometry.

Flow Cytometry

Cells from one well were counted using a Countess II Automated Cell Counter (Fisher). ˜200,000-300,000 cells from each well were added to 1.5 mL Eppendorf tubes containing PBS (final volume ˜1.2 mL). The cell suspensions were centrifuged at 1500 rpm for 7 minutes at 4° C. The supernatant was aspirated, and the cell pellet was resuspended in 99 μL of pre-chilled FACS buffer (0.5% w/v BSA in PBS). 1 μL of PE Mouse Anti-Human CD22 (5 μL/1×10⁶ cell test, BD Biosciences, Catalog No. 562859) was added and the solution was incubated at 4° C. for 30-45 minutes. Samples were then diluted with an additional 1.0 mL of FACS buffer and centrifuged at 1500 rpm for 7 minutes at 4° C. The supernatant was aspirated, the cell pellets were resuspended in 200 μL FACS buffer, and the resulting suspensions were transferred to FACS tubes (Fisher, Catalog No. 352058). Cells were stained with DAPI and CD22 surface expression was analyzed using the FACScan Analyzer at the Stanford Shared FACS Facility. Data analysis was performed using FlowJo and Microsoft Excel.

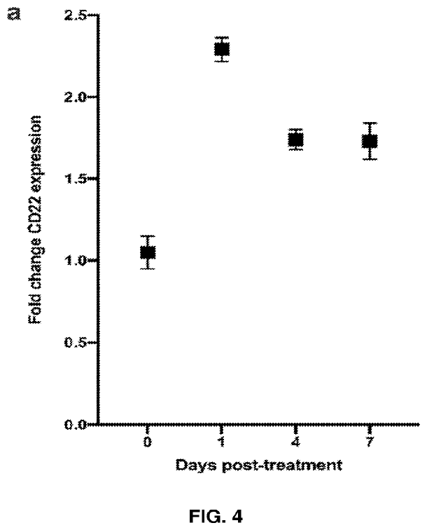

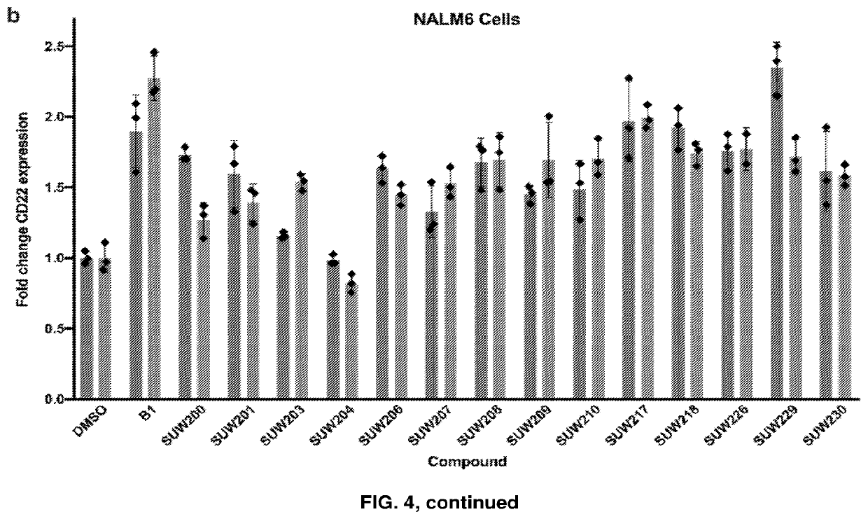

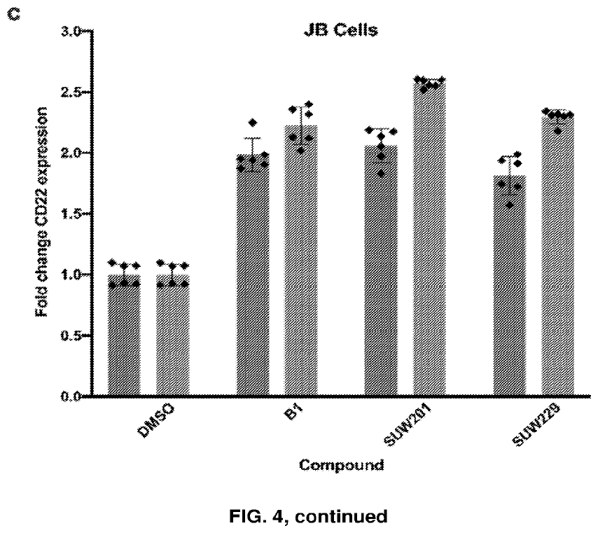

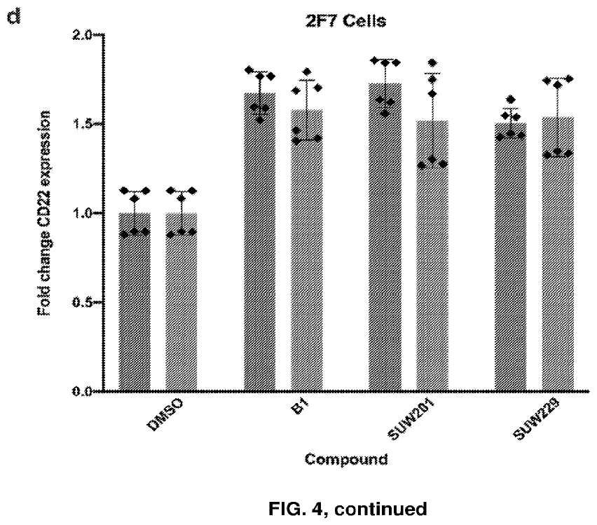

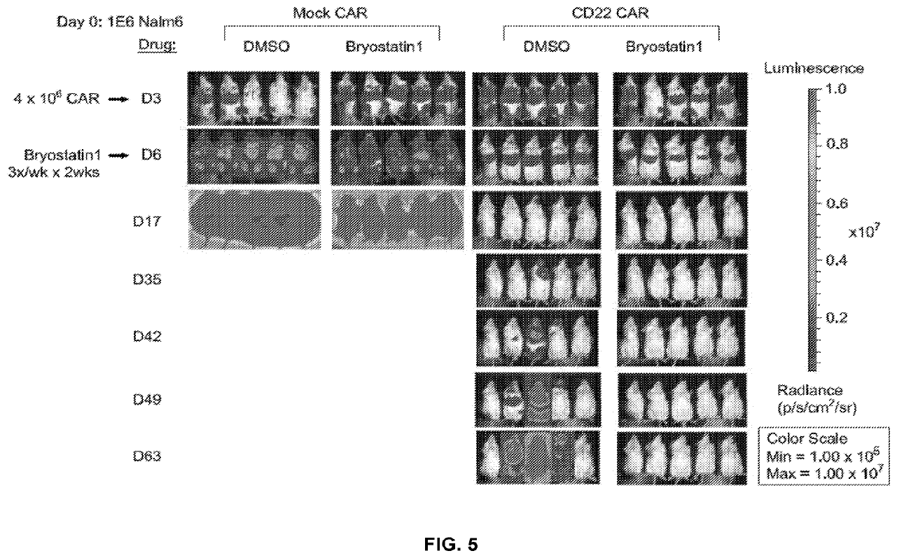

Using this assay, we sought to determine whether bryostatin 1 and bryostatin analogs could achieve sufficient upregulation of CD22 surface expression as required for CD22-targeted CAR T cell-mediated tumor clearance. NALM6 cells were incubated with bryostatin 1 and bryostatin analogs for 24 hours, at which point test compounds were washed out of the media and cells were analyzed for CD22 surface expression by flow cytometry. We found that bryostatin 1 induced a >2-fold increase in CD22 surface density (FIG. 4, panels A-B). Intriguingly, bryostatin-promoted increases in CD22 surface expression was sustained for up to 7 days following treatment (FIG. 4, panel A), suggesting that sequential administration of bryostatin 1 followed by an anti-CD22 CAR T infusion could be a viable strategy for clearing CD22^{lo} tumor cells which appear to be driving patient relapse (Fry, T. J. et al., Nat. Med. 24, 20-28 (2018)).

Importantly, while C13-modified bryostatin analogs displayed a range of activity in the CD22 induction assay, select compounds are highly effective, comparable to bryostatin 1 in our assay (FIG. 4, panel B). Surprisingly, we observed a pronounced effect in C13 enoate geometry on compound function in both the C13 methyl and allyl enoates (FIG. 4, panel B). (Z)-enoates were considerably more active than the corresponding (E)-enoates, further suggesting that PKC signaling dynamics can be modulated by modifications to the B-ring of the bryostatin scaffold. As previously noted, top performer SUW229 displays a different PKC activation profile relative to bryostatin 1, suggesting that it is possible that differential PKC activation dynamics can influence biologically significant downstream signaling outcomes.

CD22 Surface Expression Protocol—JB and 2F7 Cells

Culture Conditions

AIDS-NHL cell lines were incubated in IF10 medium, consisting of IMDM medium (Life technologies) containing 10% fetal bovine serum (FBS, Omega Scientific), 100 units/mL of penicillin, and 100 μg/ml of streptomycin (Invitrogen). Cells were incubated at 37° C. in 5% CO₂.

AIDS-NHL Cell Lines Activation Procedures

AIDS-NHL cells were cultured in a u-bottomed 96-well tissue culture plates with a cell density of 200,000 cells/well in a 200 μL volume of IF10 media containing the indicated equimolar concentration of bryostatin 1, SUW201 or SUW229. Cells were exposed to compound for the either 24 or 48 h before staining and flow cytometric analysis of receptor levels.

Flow Cytometry

Cells in each well were washed with phosphate buffered saline (PBS) containing 2% FBS, then cells were centrifuged for 9 min at 233 xg and resuspended in 50 μL of a 1:1 dilution of phosphate buffered saline (PBS): Human AB serum (Sigma). Cells were stained with antihuman CD22 (Biolegend, clone S-HCL-1, 363506) and were incubated at 4° C. for 25 minutes then cells were washed and fixed in 2% paraformaldehyde. Stained samples were stored at 4° C. Flow cytometry samples were analyzed using a FACSCelesta (BD Biosciences) flow cytometer and data were analyzed using FlowJo software (version 10).

Using this assay, to determine whether the results observed in NALM6 cells (e.g., as outlined above) apply to other cell lines, the AIDS-related lymphoma cell lines JP and 2F7 cells were incubated with synthetic bryostatin 1, SUW201 and SEQ229 (FIG. 4, panels C-D). JB is an Epstein-Barr virus (EBV)-negative AIDS-lymphoma cell line originally grown out of a bone marrow sample derived from an HIV+ individual, which harbors the Burkitt lymphoma translocation (Moses, A. V et al., Nat. Med. 3, 1242-1249 (1997)). 2F7 is an AIDS-associated non-Hodgkin's lymphoma cell line of the Burkitt subtype, which is positive for EBV (Widney, D. P. et al. Levels of Murine, but Not Human, CXCL13 Are Greatly Elevated in NOD-SCID Mice Bearing the AIDS-Associated Burkitt Lymphoma Cell Line, 2F7. PLoS One 8, e72414, https://doi.org/10.1371/journal.pone.0072414 (2013)). Burkitt lymphoma is one of the most common subtypes of AIDS non-Hodgkin's lymphoma and along with Hodgkin's lymphoma represents a significantly greater risk to AIDS patients relative to the general population due to their impaired cellular immunity (Guech-Ongey, M. et al., Blood 116, 5600-5604 (2010)). Significantly, synthetic bryostatin 1 and analogs SUW201 and SUW229 upregulated CD22 surface expression by ˜2 fold in each cell line tested (FIG. 4, panels C-D), further highlighting the potential generality of using PKC modulators for enhancing CD22-targeted cancer immunotherapies.

FIG. 4, panels A-D: Show Bryostatin-promoted cell surface expression of CD22. Panel A, illustrates that synthetic bryostatin 1 promotes increased surface expression of CD22. NALM6 cells were incubated with 10 nM bryostatin 1 for 24 hours. Compound was washed out and cells were sub-cultured for the indicated times. CD22 surface expression was then assayed by flow cytometry (n=3 biological replicates; data presented as mean values ±SE). Panel B: NALM6 cells were incubated with 1 nM (left hand bars) or 10 nM (right hand bars) compound for 24 hours. Compound was washed out and cells were sub-cultured for 24 hours. CD22 surface expression was then assayed by flow cytometry (n=3 biological replicates; data presented as mean values ±SE). Panel C: JB cells were incubated with 1 nM (left hand bars) or 10 nM (right hand bars) of compound for 48 hours. CD22 surface expression was then assayed by flow cytometry (n=6 biological replicates; data presented as mean values ±SE). Panel D: 2F7 cells were incubated with 1 nM (left hand bars) or 10 nM (right hand bars) of compound for 48 hours. CD22 surface expression was then assayed by flow cytometry (n=6 biological replicates; data presented as mean values ±SE).

This is a first of its kind collection of bryostatin analogs and includes some with binding affinities and translocation and CD activation activities similar to bryostatin while others have similarly effective affinities with some variation in selectivity and are superior CD activators and better tolerated in animal studies.

### SUMMARY

The results presented herein underscore the importance of design and chemical synthesis in natural product-inspired drug discovery. Efficient synthetic access to bryostatin 1, a compound recently thought too complex to be made in a practical fashion, has enabled access to its immediate precursors and derivatives. Most exemplary analogs show bryostatin-like PKC affinities. Other protein targets with C1 domains (e.g., as described herein) similar to PKC, can also be potential mediators of byrostatins agent activities. Some however show different affinities. Some of these exemplary analogs are synthetically more accessible, a factor that could influence candidate selection in the clinic. More importantly, several analogs are comparable to or can be better than bryostatin in translocation assays and select analogs can match or exceed the CD22 induction exhibited by bryostatin 1. Significantly, several analogs are better tolerated in animal studies. Of importance, these studies also show that bryostatin can be modified in certain regions and that while preserving affinity, these changes can result in differing translocation and CD22 induction effects.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

Notwithstanding the appended claims, the disclosure set forth herein is also described by the following clauses.

Clause 1. A method of modulating target cells in a subject, the method comprising contacting target cells with an effective amount of a bryostatin agent to selectively enhance one or more of a) expression of an antigen in the target cells, b) translocation of an antigen in the target cells, c) cell surface presentation of an antigen in the target cells, and d) cell surface persistence of an antigen in the target cells, to modulate immunogenicity of the target cells.
Clause 2. The method of clause 1, wherein the antigen is selected from a protein antigen, a peptide antigen, a neoantigen, and an antigen derived from treatment of the target cells with mRNA.
Clause 3. The method of clause 1 or 2, wherein the target cells are chimeric antigen receptor (CAR)-modified T cells or chimeric antigen receptor-natural killer cells (CAR-NK cells), and the contacting target cells with the bryostatin agent enhances expression or cell surface presentation and persistence of the CAR.
Clause 4. The method of clause 3, wherein the CAR has affinity for a target cell surface antigen selected from viral antigen, bacterial antigen, parasitic antigen, tumor cell associated antigen (TAA), disease cell associated antigen, and an antigen derived from the treatment of the cells with mRNA, and any fragment thereof.
Clause 5. The method of clause 4, wherein the modified T cells are obtained from peripheral blood mononuclear cells, cord blood cells, a purified population of T cells, or a T cell line.
Clause 6. The method of any one of clauses 3 to 5, wherein the contacting step is performed ex vivo and the target cells are derived from the subject (autologous cells).
Clause 7. The method of any one of clauses 3 to 5, wherein the contacting step is performed ex vivo and the target cells are derived from a donor (allogenic cells).
Clause 8. The method of clause 1, wherein the target cells are selected from cancer cells, cancer stem cells, and cancer progenitor cells.
Clause 9. The method of clause 8, wherein the contacting step is performed in vivo and comprises administering the bryostatin agent to a subject having cancer.
Clause 10. The method of clause 8 or 9, wherein the method sensitizes the target cells to clearance by the subject's immune system.
Clause 11. The method of clause 8, wherein the subject is relapsed or refractory to immune cell clearance and the bryostatin agent modulates T cell-mediated immune response to the target cell population.
Clause 12. The method of any one of clauses 9-11, wherein the subject is receiving an immuno-oncology therapy.
Clause 13. The method of clause 9, further comprising administering to the subject an effective amount of a therapeutic agent that is capable of one or more of inhibiting growth of the modulated target cells, or clearing the modulated target cells.
Clause 14. The method of clause 1, wherein the target cells are HIV infected cells.
Clause 15. The method of clause 14, wherein the target cells are cells infected with latent HIV and modulating immunogenicity of the target cells comprises activating expression of HIV.
Clause 16. The method of clause 1, wherein the contacting step is performed in vivo and comprises administering the bryostatin agent to a subject diagnosed with or suspected of having HIV, wherein the contacting step is capable of having a therapeutic effect.
Clause 17. The method of clause 15, further comprising administering to the subject a therapeutically effective amount of a therapeutic that is capable of clearing the modulated target cells having activated expression of HIV.
Clause 18. A method of treating a subject for cancer, the method comprising:

a) administering to a subject an effective amount of a bryostatin agent to enhance cell surface antigen or neoantigen presentation and persistence on target cells of the subject; and

b) administering to the subject a therapeutically effective amount of a therapeutic agent that specifically binds the cell surface antigen to treat the subject for cancer.

Clause 19. The method of clause 18, wherein the subject is relapsed or refractory to targeted anticancer therapy.
Clause 20. The method of clause 18, wherein the bryostatin agent sensitizes the target cancer cells to inhibition of growth by the therapeutic agent.
Clause 21. The method of clause 18, wherein the bryostatin agent sensitizes the target cancer cells to clearance by the therapeutic agent.
Clause 22. The method of clause 18, wherein prior to step a) the target cancer cells present cell surface antigen on the target cell surface at a therapeutically ineffective level.
Clause 23. The method of clause 18, wherein the bryostatin agent enhances one or more of a) expression of cell surface antigens, b) translocation of expressed cell surface antigens to the target cell surface, and c) persistence of cell surface antigens on the target cell surface.
Clause 24. The method of clause 18, wherein the bryostatin agent enhances cell surface presentation of the cell surface antigen by 50% or more.
Clause 25. The method of clause 23 or 24, wherein cell surface antigen presentation on the target cancer cell is enhanced for 2 days or more after administration of the bryostatin agent.
Clause 26. The method of any one of clauses 18-25, wherein the therapeutic agent is selected from chimeric antigen receptor expressing T cells (CAR T-cells), CAR-natural killer cells (CAR-NK cells), an antibody agent, an antibody drug conjugate (ADC) and a bispecific antibody agent.
Clause 27. The method of clause 26, wherein step b) comprises administering to the subject a composition comprising a therapeutically effective amount of CAR T-cells or CAR-NK cells that specifically bind the cell surface antigen present on a target cell population.
Clause 28. The method of clause 27, wherein the bryostatin agent modulates T cell-mediated or NK cell-mediated immune response to the target cell population.
Clause 29. The method of any one of clauses 27-28, wherein the target cell population comprises tumor antigen selected from CD10, CD19, CD20, CD21, CD22, CD27, CD28, CD30, CD33, CD34, CD38, CD40, CD52, CD80, CD86, CD137, CDK4, CDK6, OX40 and CD340.
Clause 30. The method of any one of clauses 27-29, wherein the chimeric antigen receptor expressing T cells, or NK cells, are effective for treating B cell malignancy, CLL, ALL, B-ALL, Leukemia, Lymphoma or solid tumors.
Clause 31. The method of clause 30, wherein the solid tumors are selected from breast cancer, prostate cancer, bladder cancer, soft tissue sarcoma, lymphomas, esophageal cancer, uterine cancer, bone cancer, adrenal gland cancer, lung cancer, thyroid cancer, colon cancer, glioma, liver cancer, pancreatic cancer, renal cancer, cervical cancer, testicular cancer, head and neck cancer, ovarian cancer, neuroblastoma and melanoma.
Clause 32. The method of any one of clauses 27-31, wherein administration of the bryostatin agent is prior to administration of the therapeutically effective amount of CAR-T cells, or CAR-NK cells.
Clause 33. The method of any one of clauses 27-31, wherein administration of the bryostatin agent is simultaneous with administration of the therapeutically effective amount of CAR-T cells, or CAR-NK cells.
Clause 34. The method of any one of clauses 27-31, wherein administration of the bryostatin agent is subsequent to the administration of the therapeutically effective amount of CAR-T cells, or CAR-NK cells.
Clause 35. The method of clause 26, wherein step b) comprises administering to the subject a therapeutically effective amount of an antibody agent, ADC, bispecific antibody agent that specifically binds the cell surface antigen.
Clause 36. The method of clause 35, wherein the antibody agent comprises a human monoclonal antibody, or antigen-binding portion thereof.
Clause 37. The method of clause 35, wherein the antibody agent is an antibody that comprises a full-length antibody of an IgG1 isotype or an IgG4 isotype.
Clause 38. The method of clause 35, wherein the agent is an ADC comprising a cytotoxic agent.
Clause 39. The method of clause 38, wherein the cytotoxic agent is a cytotoxin or a radioactive agent.
Clause 40. The method of clause 39, wherein the cytotoxic agent is conjugated to an antibody of the ADC via a linker.
Clause 41. The method of clause 40, wherein the linker is selected from peptidyl linkers, hydrazine linkers and disulfide linkers.
Clause 42. The method of any one of clauses 38-41, wherein the cytotoxic agent is selected from calicheamicins, auristatins, maytansinoids, taxol derivatives and duocarmycins.
Clause 43. The method of clause 35, wherein the ADC is selected from inotuzumab ozogamicin and gemtuzumab ozogamicin.
Clause 44. The method of clause 35, wherein the agent is a bispecific antibody agent.
Clause 45. The method of clause 44, wherein the bispecific antibody is an anti-CD20/anti-CD22 bispecific antibody fusion protein or an anti-CD19/anti-CD22 bispecific antibody fusion protein.
Clause 46. The method of any one of clauses 35-45, wherein the agent is administered via a route selected from orally, ocularly, aurally, subcutaneously, intravenously, intramuscularly, intradermally, intraperitoneally and inhalation.
Clause 47. The method of any one of clauses 18-46, wherein the cancer is leukemia or B cell lymphoma.
Clause 48. The method of clause 47, wherein the B cell lymphoma is non-Hodgkin's lymphoma.
Clause 49. The method of clause 47, wherein the cancer is selected from Burkitt's lymphoma and B cell chronic lymphocytic leukemia.
Clause 50. The method of any one of clauses 18-49, wherein the cancer is melanoma, prostate cancer, breast cancer, ovarian cancer, esophageal cancer, or kidney cancer.
Clause 51. The method of any one of clauses 18-50, wherein the subject is a mammal.
Clause 52. The method of clause 51, wherein the subject is relapsed or refractory to cell surface antigen targeted therapy.
Clause 53. The method of clause 52, wherein the cell surface antigen is selected from CD10, CD19, CD20, CD21, CD22, CD27, CD28, CD30, CD33, CD34, CD38, CD40, CD52, CD80, CD86, CD137, CDK4, CDK6, OX40 and CD340.
Clause 54. The method of any one of clauses 18-53, further comprising determining the level or expression or presentation of the cell surface antigen in target cancer cells of a sample obtained from the subject.
Clause 55. The method of any one of clauses 18-54, further comprising administering at least one additional anti-cancer therapy to the patient, wherein the additional anti-cancer therapy is selected from radiation therapy, chemotherapy, immunotherapy, checkpoint inhibitors, surgery and vasculature-targeting therapy.
Clause 56. The method of any one of clauses 18-55, further comprising assessing one or more biomarkers in a sample of the subject to assay the status of the cancer.

What is claimed is:

1. A method of modulating target cells in a subject, the method comprising contacting target cells with an effective amount of a bryostatin agent to selectively enhance one or more of a) expression of an antigen in the target cells, b) translocation of an antigen in the target cells, c) cell surface presentation of an antigen in the target cells, and d) cell surface persistence of an antigen in the target cells, to modulate immunogenicity of the target cells.

2. The method of claim 1, wherein the antigen is selected from a protein antigen, a peptide antigen, a neoantigen, and an antigen derived from treatment of the target cells with mRNA.

3. The method of claim 1 or 2, wherein the target cells are chimeric antigen receptor (CAR)-modified T cells or chimeric antigen receptor-natural killer cells (CAR-NK cells), and the contacting target cells with the bryostatin agent enhances expression or cell surface presentation and persistence of the CAR.

4. The method of claim 3, wherein the contacting step is performed ex vivo and the target cells are derived from the subject (autologous cells).

5. The method claim 3, wherein the contacting step is performed ex vivo and the target cells are derived from a donor (allogenic cells).

6. The method of claim 1, wherein the target cells are selected from cancer cells, cancer stem cells, and cancer progenitor cells.

7. The method of claim 6, wherein the contacting step is performed in vivo and comprises administering the bryostatin agent to a subject having cancer.

8. The method of claim 6 or 7, wherein the method sensitizes the target cells to clearance by the subject's immune system.

9. The method of claim 7, further comprising administering to the subject an effective amount of a therapeutic agent that is capable of one or more of inhibiting growth of the modulated target cells, or clearing the modulated target cells.

10. The method of claim 1, wherein the target cells are HIV infected cells.

11. The method of claim 1, wherein the contacting step is performed in vivo and comprises administering the bryostatin agent to a subject diagnosed with or suspected of having HIV, wherein the contacting step is capable of having a therapeutic effect.

12. A method of treating a subject for cancer, the method comprising:
a) administering to a subject an effective amount of a bryostatin agent to enhance cell surface antigen or neoantigen presentation and persistence on target cells of the subject; and
b) administering to the subject a therapeutically effective amount of a therapeutic agent that specifically binds the cell surface antigen to treat the subject for cancer.

13. The method of claim 12, wherein the subject is relapsed or refractory to targeted anticancer therapy.

14. The method of claim 12, wherein prior to step a) the target cancer cells present cell surface antigen on the target cell surface at a therapeutically ineffective level.

15. The method of claim 12, wherein the bryostatin agent enhances one or more of a) expression of cell surface antigens, b) translocation of expressed cell surface antigens to the target cell surface, and c) persistence of cell surface antigens on the target cell surface.

16. The method of any one of claims 12-15, wherein the therapeutic agent is selected from chimeric antigen receptor expressing T cells (CAR T-cells), CAR-natural killer cells (CAR-NK cells), antibody agent, antibody drug conjugate (ADC) and bispecific antibody agent.

17. The method of any one of claims 12-16, wherein the cancer is leukemia or B cell lymphoma.

18. The method of any one of claims 12-16, wherein the cancer is melanoma, prostate cancer, breast cancer, ovarian cancer, esophageal cancer, or kidney cancer.

19. The method of any one of claims 12-18, further comprising determining the level or expression or presentation of the cell surface antigen in target cancer cells of a sample obtained from the subject.

20. The method of any one of claims 12-19, further comprising administering at least one additional anti-cancer therapy to the patient, wherein the additional anti-cancer therapy is selected from radiation therapy, chemotherapy, immunotherapy, checkpoint inhibitors, surgery and vasculature-targeting therapy.

21. The method of any one of claims 12-20, further comprising assessing one or more biomarkers in a sample of the subject to assay the status of the cancer.
